(19) United States
(12) Patent Application Publication
(10) Pub. No.: US 2022/0273716 A1
(43) Pub. Date: Sep. 1, 2022

(54) POPULATIONS OF NATURAL KILLER CELLS COMPRISING A CD38 CHIMERIC ANTIGEN RECEPTOR

(71) Applicant: Celularity Inc., Florham Park, NJ (US)

(72) Inventors: Srinivas SOMANCHI, San Diego, CA (US); Xuan GUO, Whitehouse Station, NJ (US); Shuyang HE, Marinsville, NJ (US); Rohit MATHUR, Edison, NJ (US); Xiaokui ZHANG, Martinsville, NJ (US); Hemlata RANA, Clifton, NJ (US); Weifang LING, Piscataway, NJ (US); James W. EDINGER, Belford, NJ (US); Robert J. HARIRI, Bernardsville, NJ (US); Andrea DIFIGLIA, Oxford, NJ (US)

(73) Assignee: Celularity Inc., Florham Park, NJ (US)

(21) Appl. No.: 17/630,079

(22) PCT Filed: Jul. 27, 2020
(86) PCT No.: PCT/US2020/043743

§ 371 Date: Jan. 25, 2022

Related U.S. Application Data
(60) Provisional application No. 62/943,709, filed on Dec. 4, 2019.
(60) Provisional application No. 62/878,731, filed on Jul. 25, 2019.

(51) Int. Cl. A61K 35/17; C12N 5/0783; C12N 15/62; C12N 15/86

(52) U.S. Cl. CPC A61K 35/17; C12N 5/0646; C12N 15/625; C12N 15/86; C12N 2740/10043; C12N 2501/2302; C12N 2501/2306; C12N 2501/2307; C12N 2501/2315; C12N 2501/145; C12N 2501/22

(57) ABSTRACT

Provided herein are methods of treating cancer in a human subject comprising administering to the subject an effective amount of human placental-derived natural killer cells comprising a CD38 chimeric antigen receptor (CAR) to the subject so as thereby to provide an effective treatment of the cancer in the subject. The cells, such as CYNK cells, can be placental CD34+ cell-derived natural killer (NK) cells. The cancers to be treated include multiple myeloma and lymphoma. The present invention also provides compositions comprising human placental-derived natural killer cells comprising a CD38 chimeric antigen receptor (CAR) or the treatment of multiple myeloma and lymphoma and methods of their use.

### 1. FIELD

Provided herein are methods of producing populations of natural killer (NK) cells and/or ILC3 cells from a population of hematopoietic stem or progenitor cells in media comprising stem cell mobilizing factors, e.g., three-stage methods of producing NK cells and/or ILC3 cells in media comprising stem cell mobilizing factors starting with hematopoietic stem or progenitor cells from cells of the placenta, for example, from placental perfusate (e.g., human placental perfusate) or other tissues, for example, umbilical cord blood or peripheral blood. Further provided herein are methods of using the placental perfusate, the NK cells and/or ILC3 cells and/or NK progenitor cells described herein, to, e.g., suppress the proliferation of tumor cells, including multiple myeloma and acute myeloid leukemia cells.

### 2. BACKGROUND

Natural killer (NK) cells exhibit innate anti-tumor activity owing to the expression of a multitude of activating and inhibitory receptors that orchestrate NK cell responses. It is thus possible to use NK cells from allogeneic sources without the risk of graft-vs-host disease¹, making them very attractive for developing “off-the-shelf” cellular therapies. The anti-tumor responses of NK cells can be further enhanced by expressing Chimeric Antigen Receptors (CARs).

Celularity has developed a GMP process for generating off-the-shelf, allogeneic human Placental Hematopoietic Stem Cell (HSC) derived Natural Killer cells (PNK). The placental HSC source is vast, and Celularity process is streamlined to yield large quantities of differentiated and activated NK cells that have been well characterized. Here, we report the development of a tumor targeted approach through engineering PNK cells to express CAR against tumor antigen CD38.

CD38 is a glycoprotein and ectoenzyme highly expressed in hematological malignancies notably in lymphoma and multiple myeloma, making it an attractive target for antibody and CAR based therapies.

Multiple myeloma (MM) is the third most common blood cancer (after lymphoma and leukemia). An estimated 30,770 new cases or 1.8% of all new cancer cases in the United States (US) in 2018 will be related to MM. MM is the fourteenth leading cause of cancer death in the US, with an estimated 12,770 deaths or 2.1% of all cancer deaths a result of MM. The 5-year survival is estimated at 50.7%. Multiple myeloma is more common in men than women and among individuals of African American decent (SEER, 2018).

MM is a disease of the elderly, with 35% being younger than 65 years of age. MINI is diagnosed based on the presence of organ damage related to the underlying malignant clone which manifests with at least one of the following: hypercalcemia, renal insufficiency, anemia and bone disease (Cavo, 2011). The proliferation of plasma cells may result in the development of extramedullary plasmacytoma (excluding solitary extramedullary plasmacytoma) to a more bone marrow invasive process leading to lytic lesions or severe osteopenia. Plasma cells are an important component of the overall immune system, therefore patients with MM are susceptible to increased incidence of and slower recovery from infections. Infections are a significant cause of morbidity and mortality (Blimark, 2015).

Newly diagnosed MM (NDMM) patients are initially treated with approximately 4 cycles of induction therapy prior to undergoing stem cell harvesting for transplant (NCCN, 2019). Therapies used in induction therapy may impact the ability of stem cell collection due to their known toxicity profile of myelosuppression and the need to collect CD34+ cells. The recommendation to harvest after a few cycles, followed with an assessment of the patient's response to induction will drive treatment either to continue with additional cycles of therapy or to proceed immediately with the autologous stem cell transplant (ASCT) (Kumar, 2009).

In general, patients who are eligible for ASCT will likely receive triple combination for induction therapy. The initial therapy may include an immunomodulating agent (IMiD), a proteasome inhibitor (PI), with steroids. The overall mechanism of each of these therapies and the synergistic value of the combination is not fully understood. These novel therapies have brought the added benefit of improved responses to therapy as well as significant improvement in post transplant outcomes compared to previous chemotherapy-based regimens (Rajkumar, 2016; Kumar, 2009).

ASCT following high-dose chemotherapy has been found to be significantly superior in terms of complete response (CR) rate, time to progression (TTP) and overall survival (OS) compared to standard dose chemotherapy for the treatment of MM (Krejci, 2009). Early natural killer (NK) cell recovery (>100/□L) at one-month post ASCT is associated with improved progression free survival (PFS) in MINI (Rueff, 2014). These observations, together with the reported safety and in-vivo proliferation results from adoptive NK cell immunotherapy in MM patients (Szmania, 2015) provide a rationale for the use of NK cell-based therapies for the treatment of MM.

Following ASCT, patients are then assessed by the response and risk stratification to start maintenance with an IMiD or PI-based regimen, and either to progression or for a designated timeframe (Rajkumar, 2014). In the CALGB (Alliance) 100104 study, 460 NDMM patients were randomized 90-100 days after ASCT to receive either lenalidomide single agent maintenance or placebo following ASCT. Patients were required to have stable disease or better following the ASCT. The primary endpoint was TTP. At the time of randomization, the adjudicated very good partial response (VGPR) or better overall response rate (ORR) was 67% for the placebo group and 55% for the lenalidomide group. Importantly crossover was permitted for the placebo group and did occur for 38% of the placebo group. 1 year post ASCT, ORR was 51% for the placebo group and 48% for the lenalidomide group. 2 year post ASCT, ORR was 27% for the placebo and 36% for the lenalidomide group (Holstein, 2017). The median TTP was 57.3 months (95% CI 44.2-73.3) for the lenalidomide group and 28.9 months (23.0-36.3) for the placebo group (hazard ratio 0.57, 95% CI 0.46-0.71; p<0.0001) not accounting for crossover. Minimal residual disease (MRD) testing was not included in this study.

The IFM 2009 comparison study evaluated upfront ASCT to lenalidomide bortezomib and dexamethasone (RVD) in the frontline setting. 700 subjects were randomly assigned to receive induction therapy with three cycles of RVD then high-dose melphalan plus stem-cell transplantation followed by either two additional cycles of RVD (n=350) or consolidation therapy with five additional cycles of RVD (n=350). Both groups received maintenance therapy with lenalidomide for 1 year. The primary end point was PFS. The ORR showed 88% vs 77% for early ASCT vs RVD respectively. This study evaluated MRD, noting that bone marrow samples were obtained after the consolidation and maintenance phases were tested for MRD by means of seven-color flow cytometry (which has a sensitivity level of 10-4). Of those who were tested for MRD, 220/278 (79%) early ASCT vs 171/265 RVD (65%) achieved MRD negativity during the course of the study. PFS was 50 months vs 36 months, however it was noted that PFS was longer for those who achieved MRD negativity across both arms. Median OS had not been met at the time of the publication, however the 4 year survival did not differ significantly at 81% vs 82% (Attal, 2017).

A review of the IFM 2009 study acquired BMA samples, using MRD by NGS with a sensitivity of <10-6 showed that MRD was a strong prognostic factor for PFS and OS. Patients that achieved MRD negativity, regardless of their treatment group (RVD vs transplant) or other risk factors, had a higher probability of a longer progression free survival. Required sampling for all subjects participating on the study was not available (n=509). Of the 127 (25%) with VGPR or better by IMWG criteria, who achieved MRD negativity at any time during the study period, 73/245 (29.8%) were treated on the transplant arm and 54/264 (20.5%) were treated on the RVD arm. Overall 90 subjects (both arms) were evaluated and found to be MRD negative prior to start of lenalidomide maintenance and 92 subjects were evaluated and found to be MRD negative after 12 months of lenalidomide maintenance. The response assessment by IMWG criteria showed maintenance therapy did improve CR rates for the MRD negative arm over the course of the 12 months of therapy. PFS was significantly prolonged in subjects with MRD negative vs MRD positive. OS was also shown to be improved in the MRD negative vs MRD positive group, however the median OS was not reached in either group (Perrot, 2016).

The prognostic impact of achieving MRD negativity is currently being investigated in multiple studies. In some of these studies, the evaluation of MRD negativity as a surrogate for PFS and/or OS are ongoing. With the clinical outcomes and, duration of response improvements, time to evaluate the potential clinical benefit of new treatments is growing in time duration. This could strongly impact successful investigations of potential therapeutics in this incurable disease. As such identifying surrogate biomarkers is imperative. The results from multiple studies do not present a clear picture, in part due to wide variances of sensitivity of the assays used over the last 10 years. These data warrant further investigation and thereby longer follow-up studies to confirm any surrogacy value. However, there is growing evidence that the achievement of MRD negativity within a line of therapy does have prognostic value, especially when evaluating at <10-6 sensitivity.

PNK-007 is an allogeneic, off the shelf cell therapy enriched for CD56+/CD3− NK cells expanded from placental CD34+ cells. These placental CD34+ cells were cultivated in the presence of cytokines including stem cell factor, thrombopoietin, Flt3 ligand, IL-7, IL-15, and IL-2 for 35 days to generate PNK-007 under cGMP standards followed by release testing. The use of PNK-007 was evaluated in a Phase I single infusion study after ASCT in MM. The study is closed to enrollment; however, subjects remain in follow-up at the time of this protocol's development.

In a Phase 1 study of PNK-007 in MM, a total of 15 subjects were treated on four treatment arms 10×106 cells/kg Day 14 with or without rhIL-2, 30×106 cells/kg Day 14 with rhIL-2 or 30×106 cells/kg Day 7 with recombinant human IL-2 (rhIL-2). rhIL-2 was administered subcutaneously at 6 million units every other day for up to 6 doses to facilitate PNK 007 expansion. Subjects received variable pre ASCT induction therapy. Of the 15 subjects included, there were 12 were newly diagnosed (ND)MM and 3 relapsed/refractory (RR)MM. The 3 RRMM subjects received 1, 2 or 5 prior lines of therapy, with 2 subjects having previous ASCT. All subjects had been exposed to IMiDs and PIs. Maintenance therapy was permitted after the Day 90-100 visit myeloma assessment.

No dose-limiting toxicity, graft vs host disease (GvHD), graft failure or graft rejection were observed. No serious adverse events (SAE) were attributable to PNK 007 and the reported adverse events (AE) were consistent with AEs related to ASCT.

Based on physician assessed responses by International Myeloma Working Group (IMWG) pre ASCT, 10/15 subjects achieved VGPR or better (1 CR and 9 VGPR), and by Day 90-100, 12/15 subjects achieved VGPR or better (5 CR or stringent complete response (sCR) and 7 VGPR). Using a validated Euro-flow MRD assay by bone marrow aspirate (BMA) with a sensitivity of 10 5, pre ASCT, 4/15 (26.7%) were MRD negative, and by Day 90-100, 10/15 (66.7%) were MRD negative. At one-year post ASCT, 4/6 (66.7%) were MRD negative, with 1 converting to MRD negative after Day 90 while on maintenance therapy, 1 inadequate sample, and 1 remaining MRD positive despite maintenance therapy. These observed clinical data warrant further evaluation of placental hematopoietic stem cells-derived NK treatment in MM.

PNK-007, previously investigated in a Phase I MM study (PNK-007-MM-001), was produced with a cryopreserved Drug Substance, which was subsequently thawed, cultured, washed, filtered, and reformulated as a fresh Drug Product Plasma-Lyte®-A solution containing 10% (weight/volume) human serum albumin (HSA). The cells were concentrated at 0.5×10⁶ cells/mL, 1.5×10⁶ cells/mL, 5×10⁶ cells/mL or 15×10⁶ cells/mL, which allowed a range of clinical doses with similar infusion volumes. PNK-007 is dosed based on subject weight (eg, 10⁶ cells/kg) so the volume of the infusion scales with the subject weight (approximately 2 mL/kg). Each unit of PNK-007 was custom filled based on the subject weight, so that a full unit delivers the appropriate cell dose.

For the 9 subjects who were allocated to receive 10×10⁶ cells/kg dose, the actual dose infused of PNK-007 ranged from 6.47×10⁸ cells to 1.08×10⁹ cells with subject weight ranges from 66.7 kg to 111.6 kg. For the 6 subjects who were allocated to receive 30×10⁶ cells/kg dose, the actual dose infused of PNK-007 ranged from 1.51×10⁹ cells to 2.92×10⁹ cells with weight ranges from 51.5 kg to 99.8 kg. All 15 subjects received a single infusion of PNK-007, with 12/15 subjects also receiving rhIL-2 to facilitate expansion. No dose limiting toxicities were experienced.

Due to supply chain constraints, logistics constraints and a need to transition to an alternative manufacturing site capable of later stage and commercial manufacturing, several changes have been implemented to the manufacturing processes for PNK-007. The results of testing based on identity, purity, viability, fold expansion during manufacturing and performance of the Drug Products using a qualified cytotoxicity assay demonstrated comparability between PNK-007 and CYNK-001.

### 3. SUMMARY

The present invention provides methods of treating cancer in a human subject comprising administering to the subject an effective amount of placental-derived natural killer cells comprising a CD38 chimeric antigen receptor (CAR) to the subject so as thereby to provide an effective treatment of the cancer in the subject. In some embodiments the placental-derived natural killer (NK) cells are CYNK cells. In some embodiments the CYNK cells are placental CD34+ cell-derived natural killer (NK) cells.

In some embodiments the CYNK cells are characterized by expression of one or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS which is lower than expression of said markers in peripheral blood natural killer cells and/or expression of one or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 which is higher than expression of said markers in peripheral blood natural killer cells.

In some embodiments the CYNK cells are characterized by expression of one or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS which is lower than expression of said markers in peripheral blood natural killer cells. In some embodiments expression of 2, 3, 4, 5, 6, 7, 8, 9, 10, or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS is lower than expression of said markers in peripheral blood natural killer cells.

In some embodiments the CYNK cells are characterized by expression of one or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 which is higher than expression of said markers in peripheral blood natural killer cells. In some embodiments expression of 2, 3, 4, 5, 6, 7, 8, 9, 10, or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 is higher than expression of said markers in peripheral blood natural killer cells.

In some embodiments the CYNK cells are prepared by the methods presented herein.

In some embodiments the cancer is multiple myeloma. In some embodiments the cancer is a lymphoma.

In some embodiments the CD38 CAR is as described in WO2019087151A1.

In some embodiments the CD38 CAR has been introduced into the NK cells by transfection. In some embodiments the CD38 CAR has been introduced into the NK cells by transduction. In some embodiments the CD38 CAR has been introduced into the NK cells by retroviral transduction. In some embodiments the CD38 CAR has been introduced into the NK cells by lentiviral transduction.

The present invention also provides compositions comprising human placental-derived natural killer cells comprising a CD38 chimeric antigen receptor (CAR) for use in the treatment of a cancer in a subject.

The present invention also provides uses of a composition comprising human placental-derived natural killer cells comprising a CD38 chimeric antigen receptor (CAR) for use in the manufacture of a medicament for treatment of a cancer in a subject.

In some embodiments wherein the cancer is multiple myeloma. In some embodiments the cancer is a lymphoma.

In some embodiments the placental-derived natural killer (NK) cells are CYNK cells. In some embodiments the CYNK cells are placental CD34+ cell-derived natural killer (NK) cells.

In some embodiments the CYNK cells are characterized by expression of one or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS which is lower than expression of said markers in peripheral blood natural killer cells and/or expression of one or more markers selected from the group consisting of NDFIP2, LINC00996, MAL, CCL1, MB, SPINK2, C15orf48, CAMK1, KLRC1, TNF SF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 which is higher than expression of said markers in peripheral blood natural killer cells.

In some embodiments the CYNK cells are characterized by expression of one or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS which is lower than expression of said markers in peripheral blood natural killer cells. In some embodiments expression of 2, 3, 4, 5, 6, 7, 8, 9, 10, or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS is lower than expression of said markers in peripheral blood natural killer cells.

In some embodiments the CYNK cells are characterized by expression of one or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 which is higher than expression of said markers in peripheral blood natural killer cells. In some embodiments expression of 2, 3, 4, 5, 6, 7, 8, 9, 10, or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 is higher than expression of said markers in peripheral blood natural killer cells.

In some embodiments the CYNK cells are prepared by the methods presented herein and/or are for the uses herein.

In some embodiments the CD38 CAR is as described in WO2019087151A1.

### Terminology

As used herein, the term CYNK are CD34+ cell-derived NK cells produced by the methods described herein. In specific embodiments, CYNK cells are placental-derived NK cells. In other specific embodiments, CYNK-001 is a specific formulation of CYNK cells.

As used herein, the terms “immunomodulatory compound” and “IiMiD™” do not encompass thalidomide.

As used herein, “lenalidomide” means 3-(4′aminoisoindoline-1′-one)-1-piperidine-2,6-dione (Chemical Abstracts Service name) or 2,6-Piperidinedione,3-(4-amino-1,3-dihydro-1-oxo-2H-isoindol-2-yl)- (International Union of Pure and Applied Chemistry (IUPAC) name). As used herein, “pomalidomide” means 4-amino-2-(2,6-dioxopiperidin-3-yl)isoindole-1,3-dione.

As used herein, “multipotent,” when referring to a cell, means that the cell has the capacity to differentiate into a cell of another cell type. In certain embodiments, “a multipotent cell” is a cell that has the capacity to grow into a subset of the mammalian body's approximately 260 cell types. Unlike a pluripotent cell, a multipotent cell does not have the capacity to form all of the cell types.

As used herein, “feeder cells” refers to cells of one type that are co-cultured with cells of a second type, to provide an environment in which the cells of the second type can be maintained, and perhaps proliferate. Without being bound by any theory, feeder cells can provide, for example, peptides, polypeptides, electrical signals, organic molecules (e.g., steroids), nucleic acid molecules, growth factors (e.g., bFGF), other factors (e.g., cytokines), and metabolic nutrients to target cells. In certain embodiments, feeder cells grow in a mono-layer.

As used herein, the “natural killer cells” or “NK cells” produced using the methods described herein, without further modification, include natural killer cells from any tissue source.

As used herein, the “ILC3 cells” produced using the methods described herein, without further modification, include ILC3 cells from any tissue source.

As used herein, “placental perfusate” means perfusion solution that has been passed through at least part of a placenta, e.g., a human placenta, e.g., through the placental vasculature, and includes a plurality of cells collected by the perfusion solution during passage through the placenta.

As used herein, “placental perfusate cells” means nucleated cells, e.g., total nucleated cells, isolated from, or isolatable from, placental perfusate.

As used herein, “tumor cell suppression,” “suppression of tumor cell proliferation,” and the like, includes slowing the growth of a population of tumor cells, e.g., by killing one or more of the tumor cells in said population of tumor cells, for example, by contacting or bringing, e.g., NK cells or an NK cell population produced using a three-stage method described herein into proximity with the population of tumor cells, e.g., contacting the population of tumor cells with NK cells or an NK cell population produced using a three-stage method described herein. In certain embodiments, said contacting takes place in vitro or ex vivo. In other embodiments, said contacting takes place in vivo.

As used herein, the term “hematopoietic cells” includes hematopoietic stem cells and hematopoietic progenitor cells.

As used herein, the “undefined component” is a term of art in the culture medium field that refers to components whose constituents are not generally provided or quantified. Examples of an “undefined component” include, without limitation, serum, for example, human serum (e.g., human serum AB) and fetal serum (e.g., fetal bovine serum or fetal calf serum).

As used herein, “+”, when used to indicate the presence of a particular cellular marker, means that the cellular marker is detectably present in fluorescence activated cell sorting over an isotype control; or is detectable above background in quantitative or semi-quantitative RT-PCR.

As used herein, “—”, when used to indicate the presence of a particular cellular marker, means that the cellular marker is not detectably present in fluorescence activated cell sorting over an isotype control; or is not detectable above background in quantitative or semi-quantitative RT-PCR.

### 4. BRIEF DESCRIPTION OF THE FIGURES

FIG. 1 shows expansion of NK cells for compounds CRL1-CRL11.

FIG. 2 shows expansion of NK cells for compounds CRL12-CRL22.

FIG. 3 shows expansion of NK cells relative to SR1 positive control.

FIG. 4 shows expansion of CD34+ cells from which the NK cells were derived.

FIG. 5 shows cytotoxicity of the expanded NK cultures.

FIG. 6 shows that PNK cells highly express genes encoding the cytotoxic machinery. FIG. 6A CYNK cells were combined with peripheral blood derived NK cells (PB-NK) at 1:1 ratio and gene expression analyzed on single cell level using 10× Genomics Chromium platform and Illumina sequencing. Bioinformatics analysis utilized 10× Genomics Cell Ranger analysis pipeline. Transcript analysis was restricted to Granzyme B (GZMB) expressing cells. FIG. 6B A representative tSNE plot depicting PNK and PB-NK cells as distinct populations. FIG. 6C tSNE plots of selected NK cell-associated genes. The data is representative of two donors.

FIG. 7 shows that PNK and PB-NK cells differentially express genes encoding NK cell receptors. The expression of selected NK cell receptor genes analyzed by real-time quantitative PCR in peripheral blood NK cells (PB-NK) and CD11a+-bead-purified PNK cells. An alternative name indicated above the histogram for selected markers. The data represents mean±SD of three donors for CYNK and PBNK cells (n=3). * p<0.05, ** p<0.005, *** p<0.001.

FIG. 8 shows the gating strategy for PB-NK and CYNK cells. CYNK and PBMC cells were thawed and stained with fluorophore-coupled antibodies targeting NK cell receptors. The figure demonstrates representative dot plots and the gating strategy for the identification of CYNK and PB-NK cells. See FIG. 9 for further characterization of the populations.

FIG. 9 shows differential expression of surface proteins on CYNK and PB-NK cells. CYNK and PB-NK cells were pre-gated as indicated in FIG. 8.

FIG. 10 shows that CYNK cells form a distinct cell population from PB-NK cells based on surface protein expression. tSNE plots demonstrating differential clustering of CYNK and PB-NK cells based on their surface markers. tSNE plots were generated of flow cytometry data using FlowJo software.

FIG. 11A is a schema showing that placental CD34+ cells were expanded and differentiated after RV transduction early in the process. FIG. 11B shows expression of CD38 CAR in process and at end of process (Median and Range, n=11).

FIG. 12A shows overall cell fold expansion: Optimized process yielding higher and comparable median fold expansion for PNK-NT and PNK-CAR38 cells (n=8). FIG. 12B shows the differentiation efficiency of PNK-CAR38 was comparable to PNK-NT. FIG. 12C shows representative flow plots demonstrating differentiation and percent CD38 CAR expression on PNK-CAR38 cells compared to PNK-NT. Staining for CAR expression was performed using recombinant CD38-his protein followed by anti-His PE antibody.

FIG. 13A shows CD38 expression on MM cell lines. FIG. 13B shows lysis of MINI cell lines by PNK-CAR38 compared to PNK-NT (Mean±Std Dev). FIG. 13C shows combined anti-Multiple Myeloma cytotoxicity of PNK-CAR38 cells compared to PNK-NT (**p<0.004). Statistical analysis is performed by Wilcoxon matched-pairs two tailed t test.

FIG. 14A shows CD38 expression on Lymphoma cell lines. FIG. 14B shows percent lysis at the indicated E:T ratios for lymphoma cell lines by PNK-CAR38 compared to PNK-NT (Mean±Std Dev). FIG. 14C shows combined Combined anti-Lymphoma cytotoxicity of PNK-CAR38 cells compared to PNK-NT (****p<0.0001). FIG. 14D shows the impact of % CD38CAR expression on cytolytic function: 2-fold serial dilution of PNK-CAR38 was performed with PNK-NT, cytotoxicity assay showed 35% CD38 CAR+ve cells were needed to lyse >50% of Daudi cells. Statistical analysis is performed by Wilcoxon matched-pairs two tailed t test.

FIG. 15A shows cytotoxicity against healthy activated T cells. The top histogram shows expression of CD38 on T cells activated 5 days prior to the assay with TransAct (T cell*). Cytotoxicity was assessed against activated T cells and Daudi cells as control. FIG. 15B shows cytotoxicity against healthy activated CD34+ CD38+ progenitor cells. CD34+CD38+ hematopoietic progenitors (from 2 unrelated donors) were tested for sensitivity to lysis by PNK-CAR38.

FIG. 16A shows the schema of the lymphoma tumor model. FIG. 16B shows bioluminescence imaging (each group n=6) showed 49% reduction in BLI indicating lower tumor burden in mice 10 days after receiving PNK-CAR38 cells compared to PBS or PNK-NT controls. FIG. 16C shows a survival curve comparing PNK-NT and PNK-CAR38 with PBS control.

### 5. DETAILED DESCRIPTION

Provided herein are novel methods of producing and expanding NK cells and/or ILC3 cells from hematopoietic cells, e.g., hematopoietic stem cells or progenitor cells. Also provided herein are methods, e.g., three-stage methods, of producing NK cell populations and/or ILC3 cell populations from hematopoietic cells, e.g., hematopoietic stem cells or progenitor cells. The hematopoietic cells (e.g., CD34+ hematopoietic stem cells) used to produce the NK cells and/or ILC3 cells, and NK cell populations and/or ILC3 cell populations, may be obtained from any source, for example, without limitation, placenta, umbilical cord blood, placental blood, peripheral blood, spleen or liver. In certain embodiments, the NK cells and/or ILC3 cells or NK cell populations and/or ILC3 cell populations are produced from expanded hematopoietic cells, e.g., hematopoietic stem cells and/or hematopoietic progenitor cells. In one embodiment, hematopoietic cells are collected from a source of such cells, e.g., placenta, for example from placental perfusate, umbilical cord blood, placental blood, peripheral blood, spleen, liver (e.g., fetal liver) and/or bone marrow.

The hematopoietic cells used to produce the NK cells and/or ILC3 cells, and NK cell populations and/or ILC3 cell populations, may be obtained from any animal species. In certain embodiments, the hematopoietic stem or progenitor cells are mammalian cells. In specific embodiments, said hematopoietic stem or progenitor cells are human cells. In specific embodiments, said hematopoietic stem or progenitor cells are primate cells. In specific embodiments, said hematopoietic stem or progenitor cells are canine cells. In specific embodiments, said hematopoietic stem or progenitor cells are rodent cells.

5.1. Hematopoietic Cells

Hematopoietic cells useful in the methods disclosed herein can be any hematopoietic cells able to differentiate into NK cells and/or ILC3 cells, e.g., precursor cells, hematopoietic progenitor cells, hematopoietic stem cells, or the like. Hematopoietic cells can be obtained from tissue sources such as, e.g., bone marrow, cord blood, placental blood, peripheral blood, liver or the like, or combinations thereof. Hematopoietic cells can be obtained from placenta. In a specific embodiment, the hematopoietic cells are obtained from placental perfusate. In one embodiment, the hematopoietic cells are not obtained from umbilical cord blood. In one embodiment, the hematopoietic cells are not obtained from peripheral blood. Hematopoietic cells from placental perfusate can comprise a mixture of fetal and maternal hematopoietic cells, e.g., a mixture in which maternal cells comprise greater than 5% of the total number of hematopoietic cells. In certain embodiments, hematopoietic cells from placental perfusate comprise at least about 90%, 95%, 98%, 99% or 99.5% fetal cells.

In another specific embodiment, the hematopoietic cells, e.g., hematopoietic stem cells or progenitor cells, from which the NK cell populations and/or ILC3 cell populations produced using a three-stage method described herein are produced, are obtained from placental perfusate, umbilical cord blood, fetal liver, mobilized peripheral blood, or bone marrow. In another specific embodiment, the hematopoietic cells, e.g., hematopoietic stem cells or progenitor cells, from which the NK cell populations and/or ILC3 cell populations produced using a three-stage method described herein are produced, are combined cells from placental perfusate and cord blood, e.g., cord blood from the same placenta as the perfusate. In another specific embodiment, said umbilical cord blood is isolated from a placenta other than the placenta from which said placental perfusate is obtained. In certain embodiments, the combined cells can be obtained by pooling or combining the cord blood and placental perfusate. In certain embodiments, the cord blood and placental perfusate are combined at a ratio of 100:1, 95:5, 90:10, 85:15, 80:20, 75:25, 70:30, 65:35, 60:40, 55:45: 50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like by volume to obtain the combined cells. In a specific embodiment, the cord blood and placental perfusate are combined at a ratio of from 10:1 to 1:10, from 5:1 to 1:5, or from 3:1 to 1:3. In another specific embodiment, the cord blood and placental perfusate are combined at a ratio of 10:1, 5:1, 3:1, 1:1, 1:3, 1:5 or 1:10. In a more specific embodiment, the cord blood and placental perfusate are combined at a ratio of 8.5:1.5 (85%:15%).

In certain embodiments, the cord blood and placental perfusate are combined at a ratio of 100:1, 95:5, 90:10, 85:15, 80:20, 75:25, 70:30, 65:35, 60:40, 55:45: 50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like by total nucleated cells (TNC) content to obtain the combined cells. In a specific embodiment, the cord blood and placental perfusate are combined at a ratio of from 10:1 to 10:1, from 5:1 to 1:5, or from 3:1 to 1:3. In another specific embodiment, the cord blood and placental perfusate are combined at a ratio of 10:1, 5:1, 3:1, 1:1, 1:3, 1:5 or 1:10.

In another specific embodiment, the hematopoietic cells, e.g., hematopoietic stem cells or progenitor cells from which said NK cell populations and/or ILC3 cell populations produced using a three-stage method described herein are produced, are from both umbilical cord blood and placental perfusate, but wherein said umbilical cord blood is isolated from a placenta other than the placenta from which said placental perfusate is obtained.

In certain embodiments, the hematopoietic cells are CD34⁺ cells. In specific embodiments, the hematopoietic cells useful in the methods disclosed herein are CD34⁺CD38⁺ or CD34⁺CD38⁻. In a more specific embodiment, the hematopoietic cells are CD34⁺CD38⁻Lin⁻. In another specific embodiment, the hematopoietic cells are one or more of CD2⁻, CD3⁻, CD11b⁻, CD11c⁻, CD14⁻, CD16⁻, CD19⁻, CD24⁻, CD56⁻, CD66b⁻ and/or glycophorin A⁻. In another specific embodiment, the hematopoietic cells are CD2⁻, CD3⁻, CD11b⁻, CD11c⁻, CD14⁻, CD16⁻, CD19⁻, CD24⁻, CD56⁻, CD66b⁻ and glycophorin A⁻. In another more specific embodiment, the hematopoietic cells are CD34⁺CD38⁻CD33⁻CD117⁻. In another more specific embodiment, the hematopoietic cells are CD34⁺CD38⁻CD33⁻CD117⁻CD235⁻CD36⁻.

In another embodiment, the hematopoietic cells are CD45⁺. In another specific embodiment, the hematopoietic cells are CD34⁺CD45⁺. In another embodiment, the hematopoietic cell is Thy-1⁺. In a specific embodiment, the hematopoietic cell is CD34⁺ Thy-1⁺. In another embodiment, the hematopoietic cells are CD133⁺. In specific embodiments, the hematopoietic cells are CD34⁺CD133⁺ or CD133⁺ Thy-1⁺. In another specific embodiment, the CD34⁺ hematopoietic cells are CXCR4⁺. In another specific embodiment, the CD34⁺ hematopoietic cells are CXCR4⁻. In another embodiment, the hematopoietic cells are positive for KDR (vascular growth factor receptor 2). In specific embodiments, the hematopoietic cells are CD34⁺KDR⁺, CD133⁺KDR⁺ or Thy-1⁺KDR⁺. In certain other embodiments, the hematopoietic cells are positive for aldehyde dehydrogenase (ALDH⁺), e.g., the cells are CD34⁺ALDH⁺.

In certain other embodiments, the CD34⁺ cells are CD45⁻. In specific embodiments, the CD34⁺ cells, e.g., CD34⁺, CD45⁻ cells express one or more, or all, of the miRNAs hsa-miR-380, hsa-miR-512, hsa-miR-517, hsa-miR-518c, hsa-miR-519b, hsa-miR-520a, hsa-miR-337, hsa-miR-422a, hsa-miR-549, and/or hsa-miR-618.

In certain embodiments, the hematopoietic cells are CD34⁻.

The hematopoietic cells can also lack certain markers that indicate lineage commitment, or a lack of developmental naiveté. For example, in another embodiment, the hematopoietic cells are HLA-DR⁻. In specific embodiments, the hematopoietic cells are CD34⁺HLA-DR⁻, CD133⁺HLA-DR⁻, Thy-1⁺HLA-DR⁻ or ALDWHLA-DR⁻ In another embodiment, the hematopoietic cells are negative for one or more, or all, of lineage markers CD2, CD3, CD11b, CD11c, CD14, CD16, CD19, CD24, CD56, CD66b and glycophorin A.

Thus, hematopoietic cells can be selected for use in the methods disclosed herein on the basis of the presence of markers that indicate an undifferentiated state, or on the basis of the absence of lineage markers indicating that at least some lineage differentiation has taken place. Methods of isolating cells, including hematopoietic cells, on the basis of the presence or absence of specific markers is discussed in detail below.

Hematopoietic cells used in the methods provided herein can be a substantially homogeneous population, e.g., a population comprising at least about 95%, at least about 98% or at least about 99% hematopoietic cells from a single tissue source, or a population comprising hematopoietic cells exhibiting the same hematopoietic cell-associated cellular markers. For example, in various embodiments, the hematopoietic cells can comprise at least about 95%, 98% or 99% hematopoietic cells from bone marrow, cord blood, placental blood, peripheral blood, or placenta, e.g., placenta perfusate.

Hematopoietic cells used in the methods provided herein can be obtained from a single individual, e.g., from a single placenta, or from a plurality of individuals, e.g., can be pooled. Where the hematopoietic cells are obtained from a plurality of individuals and pooled, the hematopoietic cells may be obtained from the same tissue source. Thus, in various embodiments, the pooled hematopoietic cells are all from placenta, e.g., placental perfusate, all from placental blood, all from umbilical cord blood, all from peripheral blood, and the like.

Hematopoietic cells used in the methods disclosed herein can, in certain embodiments, comprise hematopoietic cells from two or more tissue sources. For example, in certain embodiments, when hematopoietic cells from two or more sources are combined for use in the methods herein, a plurality of the hematopoietic cells used to produce natural killer cells using a three-stage method described herein comprise hematopoietic cells from placenta, e.g., placenta perfusate. In various embodiments, the hematopoietic cells used to produce NK cell populations and/or ILC3 cell populations produced using a three-stage method described herein, comprise hematopoietic cells from placenta and from cord blood; from placenta and peripheral blood; from placenta and placental blood, or placenta and bone marrow. In one embodiment, the hematopoietic cells comprise hematopoietic cells from placental perfusate in combination with hematopoietic cells from cord blood, wherein the cord blood and placenta are from the same individual, i.e., wherein the perfusate and cord blood are matched. In embodiments in which the hematopoietic cells comprise hematopoietic cells from two tissue sources, the hematopoietic cells from the sources can be combined in a ratio of, for example, 1:10, 2:9, 3:8, 4:7, 5:6, 6:5, 7:4, 8:3, 9:2, 1:10, 1:9, 1:8, 1:7, 1:6, 1:5, 1:4, 1:3, 1:2, 1:1, 2:1, 3:1, 4:1, 5:1, 6:1, 7:1, 8:1 or 9:1.

5.1.1. Placental Hematopoietic Stem Cells

In certain embodiments, the hematopoietic cells used in the methods provided herein are placental hematopoietic cells. In one embodiment, placental hematopoietic cells are CD34⁺. In a specific embodiment, the placental hematopoietic cells are predominantly (e.g., at least about 50%, 55%, 60%, 65%, 70%, 75%, 80%, 85%, 90%, 95% or 98%) CD34+CD38⁻ cells. In another specific embodiment, the placental hematopoietic cells are predominantly (e.g., at least about 50%, 55%, 60%, 65%, 70%, 75%, 80%, 85%, 90%, 95% or 98%) CD34⁺CD38⁺ cells. Placental hematopoietic cells can be obtained from a post-partum mammalian (e.g., human) placenta by any means known to those of skill in the art, e.g., by perfusion.

In another embodiment, the placental hematopoietic cell is CD45⁻. In a specific embodiment, the hematopoietic cell is CD34⁺CD45⁻. In another specific embodiment, the placental hematopoietic cells are CD34⁺CD45⁺.

5.2. Production of Natural Killer and/or ILC3 Cells and Natural Killer Cell and/or ILC3 Cell Populations

Production of NK cells and/or ILC3 cells and NK cell and/or ILC3 cell populations by the present methods comprises expanding a population of hematopoietic cells. During cell expansion, a plurality of hematopoietic cells within the hematopoietic cell population differentiate into NK cells and/or ILC3 cells. In one aspect, provided herein is a method of producing NK cells comprising culturing hematopoietic stem cells or progenitor cells, e.g., CD34⁺ stem cells or progenitor cells, in a first medium comprising a stem cell mobilizing agent and thrombopoietin (Tpo) to produce a first population of cells, subsequently culturing said first population of cells in a second medium comprising a stem cell mobilizing agent and interleukin-15 (IL-15), and lacking Tpo, to produce a second population of cells, and subsequently culturing said second population of cells in a third medium comprising IL-2 and IL-15, and lacking a stem cell mobilizing agent and LMWH, to produce a third population of cells, wherein the third population of cells comprises natural killer cells that are CD56+, CD3−, and wherein at least 70%, for example at least 80%, of the natural killer cells are viable. In certain embodiments, such natural killer cells comprise natural killer cells that are CD16−. In certain embodiments, such natural killer cells comprise natural killer cells that are CD94+. In certain embodiments, such natural killer cells comprise natural killer cells that are CD94+ or CD16+. In certain embodiments, such natural killer cells comprise natural killer cells that are CD94− or CD16−. In certain embodiments, such natural killer cells comprise natural killer cells that are CD94+ and CD16+. In certain embodiments, such natural killer cells comprise natural killer cells that are CD94− and CD16−. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing NK cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking LMWH, to produce a third population of cells; wherein the third population of cells comprises natural killer cells that are CD56+, CD3−, and CD11a+. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing NK cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking each of stem cell factor (SCF) and LMWH, to produce a third population of cells; wherein the third population of cells comprises natural killer cells that are CD56+, CD3−, and CD11a+. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing NK cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking each of SCF, a stem cell mobilizing agent, and LMWH, to produce a third population of cells; wherein the third population of cells comprises natural killer cells that are CD56+, CD3−, and CD11a+. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing NK cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; (c) culturing the second population of cells in a third medium comprising IL-2 and IL-15, and lacking each of a stem cell mobilizing agent and LMWH, to produce a third population of cells; and (d) isolating CD11a+ cells from the third population of cells to produce a fourth population of cells; wherein the fourth population of cells comprises natural killer cells that are CD56+, CD3−, and CD11a+. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In certain embodiments, of any of the above embodiments, said natural killer cells express perforin and EOMES. In certain embodiments, said natural killer cells do not express either RORγt or IL1R1.

In one aspect, provided herein is a method of producing ILC3 cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking LMWH, to produce a third population of cells; wherein the third population of cells comprises ILC3 cells that are CD56+, CD3−, and CD11a−. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing ILC3 cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising a stem cell mobilizing agent, IL-2 and IL-15, and lacking LMWH, to produce a third population of cells; wherein the third population of cells comprises ILC3 cells that are CD56+, CD3−, and CD11a−. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing ILC3 cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising SCF, IL-2 and IL-15, and lacking LMWH, to produce a third population of cells; wherein the third population of cells comprises ILC3 cells that are CD56+, CD3−, and CD11a−. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing ILC3 cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising a stem cell mobilizing agent, SCF, IL-2 and IL-15, and lacking LMWH, to produce a third population of cells; wherein the third population of cells comprises ILC3 cells that are CD56+, CD3−, and CD11a−. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing ILC3 cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; (c) culturing the second population of cells in a third medium comprising IL-2 and IL-15, and lacking each of a stem cell mobilizing agent and LMWH, to produce a third population of cells; and (d) isolating CD11a− cells, or removing CD11a+ cells, from the third population of cells to produce a fourth population of cells; wherein the fourth population of cells comprises ILC3 cells that are CD56+, CD3−, and CD11a−. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In certain embodiments, said ILC3 cells express RORγt and IL1R1. In certain embodiments, said ILC3 cells do not express either perforin or EOMES.

5.2.1. Production of NK Cell and/or ILC3 Cell Populations Using a Three-Stage Method

In one embodiment, provided herein is a three-stage method of producing NK cell and/or ILC3 cell populations. In certain embodiments, the method of expansion and differentiation of the hematopoietic cells, as described herein, to produce NK cell and/or ILC3 cell populations according to a three-stage method described herein comprises maintaining the cell population comprising said hematopoietic cells at between about 2×10⁴ and about 6×10⁶ cells per milliliter. In certain aspects, said hematopoietic stem or progenitor cells are initially inoculated into said first medium from 1×10⁴ to 1×10⁵ cells/mL. In a specific aspect, said hematopoietic stem or progenitor cells are initially inoculated into said first medium at about 3×10⁴ cells/mL.

In certain aspects, said first population of cells are initially inoculated into said second medium from 5×10⁴ to 5×10⁵ cells/mL. In a specific aspect, said first population of cells is initially inoculated into said second medium at about 1×10⁵ cells/mL.

In certain aspects said second population of cells is initially inoculated into said third medium from 1×10⁵ to 5×10⁶ cells/mL. In certain aspects, said second population of cells is initially inoculated into said third medium from 1×10⁵ to 1×10⁶ cells/mL. In a specific aspect, said second population of cells is initially inoculated into said third medium at about 5×10⁵ cells/mL. In a more specific aspect, said second population of cells is initially inoculated into said third medium at about 5×10⁵ cells/mL in a spinner flask. In a specific aspect, said second population of cells is initially inoculated into said third medium at about 3×10⁵ cells/mL. In a more specific aspect, said second population of cells is initially inoculated into said third medium at about 3×10⁵ cells/mL in a static culture.

In a certain embodiment, the three-stage method comprises a first stage (“stage 1”) comprising culturing hematopoietic stem cells or progenitor cells, e.g., CD34⁺ stem cells or progenitor cells, in a first medium for a specified time period, e.g., as described herein, to produce a first population of cells. In certain embodiments, the first medium comprises a stem cell mobilizing agent and thrombopoietin (Tpo). In certain embodiments, the first medium comprises in addition to a stem cell mobilizing agent and Tpo, one or more of LMWH, Flt-3L, SCF, IL-6, IL-7, G-CSF, and GM-CSF. In a specific embodiment, the first medium comprises in addition to a stem cell mobilizing agent and Tpo, each of LMWH, Flt-3L, SCF, IL-6, IL-7, G-CSF, and GM-CSF. In a specific embodiment, the first medium lacks added LMWH. In a specific embodiment, the first medium lacks added desulphated glycosaminoglycans. In a specific embodiment, the first medium lacks LMWH. In a specific embodiment, the first medium lacks desulphated glycosaminoglycans. In a specific embodiment, in addition to a stem cell mobilizing agent and Tpo, each of Flt-3L, SCF, IL-6, IL-7, G-CSF, and GM-CSF. In specific embodiments, the first medium lacks leukemia inhibiting factor (LIF), macrophage inhibitory protein-1alpha (MIP-1α) or both.

In certain embodiments, subsequently, in “stage 2” said cells are cultured in a second medium for a specified time period, e.g., as described herein, to produce a second population of cells. In certain embodiments, the second medium comprises a stem cell mobilizing agent and interleukin-15 (IL-15) and lacks Tpo. In certain embodiments, the second medium comprises, in addition to a stem cell mobilizing agent and IL-15, one or more of LMWH, Flt-3, SCF, IL-6, IL-7, G-CSF, and GM-CSF. In certain embodiments, the second medium comprises, in addition to a stem cell mobilizing agent and IL-15, each of LMWH, Flt-3, SCF, IL-6, IL-7, G-CSF, and GM-CSF. In a specific embodiment, the second medium lacks added LMWH. In a specific embodiment, the second medium lacks added desulphated glycosaminoglycans. In a specific embodiment, the second medium lacks heparin, e.g., LMWH. In a specific embodiment, the second medium lacks desulphated glycosaminoglycans. In certain embodiments, the second medium comprises, in addition to a stem cell mobilizing agent and IL-15, each of Flt-3, SCF, IL-6, IL-7, G-CSF, and GM-CSF. In specific embodiments, the second medium lacks leukemia inhibiting factor (LIF), macrophage inhibitory protein-1alpha (MIP-1α) or both.

In certain embodiments, subsequently, in “stage 3” said cells are cultured in a third medium for a specified time period, e.g., as described herein, to produce a third population of cell, e.g., natural killer cells. In certain embodiments, the third medium comprises IL-2 and IL-15, and lacks a stem cell mobilizing agent and LMWH. In certain embodiments, the third medium comprises in addition to IL-2 and IL-15, one or more of SCF, IL-6, IL-7, G-CSF, and GM-CSF. In certain embodiments, the third medium comprises, in addition to IL-2 and IL-15, each of SCF, IL-6, IL-7, G-CSF, and GM-CSF. In specific embodiments, the first medium lacks one, two, or all three of LIF, MIP-1α, and Flt3L. In specific embodiments, the third medium lacks added desulphated glycosaminoglycans. In specific embodiments, the third medium lacks desulphated glycosaminoglycans. In specific embodiments, the third medium lacks heparin, e.g., LMWH.

In a specific embodiment, the three-stage method is used to produce NK cell and/or ILC3 cell populations. In certain embodiments, the three-stage method is conducted in the absence of stromal feeder cell support. In certain embodiments, the three-stage method is conducted in the absence of exogenously added steroids (e.g., cortisone, hydrocortisone, or derivatives thereof).

In certain aspects, said first medium used in the three-stage method comprises a stem cell mobilizing agent and thrombopoietin (Tpo). In certain aspects, the first medium used in the three-stage method comprises, in addition to a stem cell mobilizing agent and Tpo, one or more of Low Molecular Weight Heparin (LMWH), Flt-3 Ligand (Flt-3L), stem cell factor (SCF), IL-6, IL-7, granulocyte colony-stimulating factor (G-CSF), or granulocyte-macrophage-stimulating factor (GM-CSF). In certain aspects, the first medium used in the three-stage method comprises, in addition to a stem cell mobilizing agent and Tpo, each of LMWH, Flt-3L, SCF, IL-6, IL-7, G-CSF, and GM-CSF. In certain aspects, the first medium used in the three-stage method comprises, in addition to a stem cell mobilizing agent and Tpo, each of Flt-3L, SCF, IL-6, IL-7, G-CSF, and GM-CSF. In a specific aspect, the first medium lacks added LMWH. In a specific aspect, the first medium lacks added desulphated glycosaminoglycans. In a specific aspect, the first medium lacks LMWH. In a specific aspect, the first medium lacks desulphated glycosaminoglycans. In certain aspects, said Tpo is present in the first medium at a concentration of from 1 ng/mL to 100 ng/mL, from 1 ng/mL to 50 ng/mL, from 20 ng/mL to 30 ng/mL, or about 25 ng/mL. In other aspects, said Tpo is present in the first medium at a concentration of from 100 ng/mL to 500 ng/mL, from 200 ng/mL to 300 ng/mL, or about 250 ng/mL. In certain aspects, when LMWH is present in the first medium, the LMWH is present at a concentration of from 1 U/mL to 10 U/mL; the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, in the first medium, the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, when LMWH is present in the first medium, the LMWH is present at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in the first medium, the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, when LMWH is present in the first medium, the LMWH is present at a concentration of about 4.5 U/mL; the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL. In certain aspects, in the first medium, the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL. In certain embodiments, said first medium additionally comprises one or more of the following: antibiotics such as gentamycin; antioxidants such as transferrin, insulin, and/or beta-mercaptoethanol; sodium selenite; ascorbic acid; ethanolamine; and glutathione. In certain embodiments, the medium that provides the base for the first medium is a cell/tissue culture medium known to those of skill in the art, e.g., a commercially available cell/tissue culture medium such as SCGM™, STEMMACS™, GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640; or is a medium that comprises components generally included in known cell/tissue culture media, such as the components included in GBGM®, AIM-V®, X-VIVO 10, X-VIVO™ 15, OPTMIZER, STEMSPAN® H3000, CELLGRO COMPLETE″, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640. In certain embodiments, said first medium is not GBGM®. In specific embodiments of any of the above embodiments, the first medium lacks LIF, MIP-1α, or both.

In certain aspects, said second medium used in the three-stage method comprises a stem cell mobilizing agent and interleukin-15 (IL-15), and lacks Tpo. In certain aspects, the second medium used in the three-stage method comprises, in addition to a stem cell mobilizing agent and IL-15, one or more of LMWH, Flt-3, SCF, IL-6, IL-7, G-CSF, and GM-CSF. In certain aspects, the second medium used in the three-stage method comprises, in addition to a stem cell mobilizing agent and IL-15, each of LMWH, Flt-3, SCF, IL-6, IL-7, G-CSF, and GM-CSF. In certain aspects, the second medium used in the three-stage method comprises, in addition to a stem cell mobilizing agent and IL-15, each of Flt-3, SCF, IL-6, IL-7, G-CSF, and GM-CSF. In a specific aspect, the second medium lacks added LMWH. In a specific aspect, the second medium lacks added desulphated glycosaminoglycans. In a specific aspect, the second medium lacks LMWH. In a specific aspect, the second medium lacks desulphated glycosaminoglycans. In certain aspects, said IL-15 is present in said second medium at a concentration of from 1 ng/mL to 50 ng/mL, from 10 ng/mL to 30 ng/mL, or about 20 ng/mL. In certain aspects, when LMWH is present in said second medium, the LMWH is present at a concentration of from 1 U/mL to 10 U/mL; the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, in said second medium, the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, when LMWH is present in the second medium, the LMWH is present in the second medium at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in the second medium, the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, when LMWH is present in the second medium, the LMWH is present in the second medium at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in the second medium, the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, when LMWH is present in the second medium, the LMWH is present in the second medium at a concentration of about 4.5 U/mL; the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL. In certain aspects, in the second medium, the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL. In certain embodiments, said second medium additionally comprises one or more of the following: antibiotics such as gentamycin; antioxidants such as transferrin, insulin, and/or beta-mercaptoethanol; sodium selenite; ascorbic acid; ethanolamine; and glutathione. In certain embodiments, the medium that provides the base for the second medium is a cell/tissue culture medium known to those of skill in the art, e.g., a commercially available cell/tissue culture medium such as SCGM™, STEMMACS™, GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640; or is a medium that comprises components generally included in known cell/tissue culture media, such as the components included in GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640. In certain embodiments, said second medium is not GBGM®. In specific embodiments of any of the above embodiments, the first medium lacks LIF, MIP-1α, or both.

In certain aspects, said third medium used in the three-stage method comprises IL-2 and IL-15, and lacks a stem cell mobilizing agent and LMWH. In certain aspects, said third medium used in the three-stage method comprises IL-2 and IL-15, and lacks LMWH. In certain aspects, said third medium used in the three-stage method comprises IL-2 and IL-15, and lacks SCF and LMWH. In certain aspects, said third medium used in the three-stage method comprises IL-2 and IL-15, and lacks SCF, a stem cell mobilizing agent and LMWH. In certain aspects, said third medium used in the three-stage method comprises a stem cell mobilizing agent, IL-2 and IL-15, and lacks LMWH. In certain aspects, said third medium used in the three-stage method comprises SCF, IL-2 and IL-15, and lacks LMWH. In certain aspects, said third medium used in the three-stage method comprises a stem cell mobilizing agent, SCF, IL-2 and IL-15, and lacks LMWH. In certain aspects, said third medium used in the three-stage method comprises IL-2 and IL-15, and lacks a stem cell mobilizing agent and LMWH. In certain aspects, the third medium used in the three-stage method comprises, in addition to IL-2 and IL-15, one or more of SCF, IL-6, IL-7, G-CSF, or GM-CSF. In certain aspects, the third medium used in the three-stage method comprises, in addition to IL-2 and IL-15, each of SCF, IL-6, IL-7, G-CSF, and GM-CSF. In certain aspects, said IL-2 is present in said third medium at a concentration of from 10 U/mL to 10,000 U/mL and said IL-15 is present in said third medium at a concentration of from 1 ng/mL to 50 ng/mL. In certain aspects, said IL-2 is present in said third medium at a concentration of from 100 U/mL to 10,000 U/mL and said IL-15 is present in said third medium at a concentration of from 1 ng/mL to 50 ng/mL. In certain aspects, said IL-2 is present in said third medium at a concentration of from 300 U/mL to 3,000 U/mL and said IL-15 is present in said third medium at a concentration of from 10 ng/mL to 30 ng/mL. In certain aspects, said IL-2 is present in said third medium at a concentration of about 1,000 U/mL and said IL-15 is present in said third medium at a concentration of about 20 ng/mL. In certain aspects, in said third medium, the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, in said third medium, the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in said third medium, the SCF is present at a concentration of about 22 ng/mL; the IL-6 is present at a concentration of about 0.05 ng/mL; the IL-7 is present at a concentration of about 20 ng/mL; the G-CSF is present at a concentration of about 0.25 ng/mL; and the GM-CSF is present at a concentration of about 0.01 ng/mL. In certain aspects, the third medium comprises 100 ng/mL IL-7, 1000 ng/mL IL-2, 20 ng/mL IL-15, and 10 stem cell mobilizing agent and lacks SCF. In certain aspects, the third medium comprises 20 ng/mL IL-7, 1000 ng/mL IL-2, 20 ng/mL IL-15, and stem cell mobilizing agent and lacks SCF. In certain aspects, the third medium comprises 20 ng/mL IL-7, 20 ng/mL IL-15, and stem cell mobilizing agent and lacks SCF. In certain aspects, the third medium comprises 100 ng/mL IL-7, 22 ng/mL SCF, 1000 ng/mL IL-2, and 20 ng/mL IL-15 and lacks stem cell mobilizing agent. In certain aspects, the third medium comprises 22 ng/mL SCF, 1000 ng/mL IL-2, and 20 ng/mL IL-15 and lacks stem cell mobilizing agent. In certain aspects, the third medium comprises 20 ng/mL IL-7, 22 ng/mL SCF, 1000 ng/mL IL-2, and 20 ng/mL IL-15 and lacks stem cell mobilizing agent. In certain aspects, the third medium comprises 20 ng/mL IL-7, 22 ng/mL SCF, and 1000 ng/mL IL-2 and lacks stem cell mobilizing agent. In specific embodiments of any of the above embodiments, the first medium lacks one, two, or all three of LIF, MIP-1α, Flt-3L.

In certain embodiments, said third medium additionally comprises one or more of the following: antibiotics such as gentamycin; antioxidants such as transferrin, insulin, and/or beta-mercaptoethanol; sodium selenite; ascorbic acid; ethanolamine; and glutathione. In certain embodiments, the medium that provides the base for the third medium is a cell/tissue culture medium known to those of skill in the art, e.g., a commercially available cell/tissue culture medium such as SCGM™, STEMMACS™, GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640; or is a medium that comprises components generally included in known cell/tissue culture media, such as the components included in GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640. In certain embodiments, said third medium is not GBGM®.

Generally, the particularly recited medium components do not refer to possible constituents in an undefined component of said medium. For example, said Tpo, IL-2, and IL-15 are not comprised within an undefined component of the first medium, second medium or third medium, e.g., said Tpo, IL-2, and IL-15 are not comprised within serum. Further, said LMWH, Flt-3, SCF, IL-6, IL-7, G-CSF, and/or GM-CSF are not comprised within an undefined component of the first medium, second medium or third medium, e.g., said LMWH, Flt-3, SCF, IL-6, IL-7, G-CSF, and/or GM-CSF are not comprised within serum.

In certain aspects, said first medium, second medium or third medium comprises human serum-AB. In certain aspects, any of said first medium, second medium or third medium comprises 1% to 20% human serum-AB, 5% to 15% human serum-AB, or about 2, 5, or 10% human serum-AB.

In certain embodiments, in the three-stage methods described herein, said hematopoietic stem or progenitor cells are cultured in said first medium for 1, 2, 3, 4, 5, 6, 7, 8, 9, 10, 11, 12, 13, 14, 15, 16, 17, 18, 19, or 20 days. In certain embodiments, in the three-stage methods described herein, cells are cultured in said second medium for 1, 2, 3, 4, 5, 6, 7, 8, 9, 10, 11, 12, 13, 14, 15, 16, 17, 18, 19, or 20 days. In certain embodiments, in the three-stage methods described herein, cells are cultured in said third medium for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or for more than 30 days.

In a specific embodiment, in the three-stage methods described herein, said hematopoietic stem or progenitor cells are cultured in said first medium for 7-13 days to produce a first population of cells, before said culturing in said second medium; said first population of cells are cultured in said second medium for 2-6 days to produce a second population of cells before said culturing in said third medium; and said second population of cells are cultured in said third medium for 10-30 days, i.e., the cells are cultured a total of 19-49 days.

In a specific embodiment, in the three-stage methods described herein, in the three-stage methods described herein, said hematopoietic stem or progenitor cells are cultured in said first medium for 8-12 days to produce a first population of cells, before said culturing in said second medium; said first population of cells are cultured in said second medium for 3-5 days to produce a second population of cells before said culturing in said third medium; and said second population of cells are cultured in said third medium for 15-25 days, i.e., the cells are cultured a total of 26-42 days.

In a specific embodiment, in the three-stage methods described herein, said hematopoietic stem or progenitor cells are cultured in said first medium for about 10 days to produce a first population of cells, before said culturing in said second medium; said first population of cells are cultured in said second medium for about 4 days to produce a second population of cells before said culturing in said third medium; and said second population of cells are cultured in said third medium for about 21 days, i.e., the cells are cultured a total of about 35 days.

In certain aspects, the three-stage method disclosed herein produces at least 5000-fold more natural killer cells as compared to the number of hematopoietic stem cells initially inoculated into said first medium. In certain aspects, said three-stage method produces at least 10,000-fold more natural killer cells as compared to the number of hematopoietic stem cells initially inoculated into said first medium. In certain aspects, said three-stage method produces at least 50,000-fold more natural killer cells as compared to the number of hematopoietic stem cells initially inoculated into said first medium. In certain aspects, said three-stage method produces at least 75,000-fold more natural killer cells as compared to the number of hematopoietic stem cells initially inoculated into said first medium. In certain aspects, the viability of said natural killer cells is determined by 7-aminoactinomycin D (7AAD) staining. In certain aspects, the viability of said natural killer cells is determined by annexin-V staining. In specific aspects, the viability of said natural killer cells is determined by both 7-AAD staining and annexin-V staining. In certain aspects, the viability of said natural killer cells is determined by trypan blue staining.

In certain aspects, the three-stage method disclosed herein produces at least 5000-fold more ILC3 cells as compared to the number of hematopoietic stem cells initially inoculated into said first medium. In certain aspects, said three-stage method produces at least 10,000-fold more ILC3 cells as compared to the number of hematopoietic stem cells initially inoculated into said first medium. In certain aspects, said three-stage method produces at least 50,000-fold more ILC3 cells as compared to the number of hematopoietic stem cells initially inoculated into said first medium. In certain aspects, said three-stage method produces at least 75,000-fold more ILC3 cells as compared to the number of hematopoietic stem cells initially inoculated into said first medium.

In certain aspects, the three-stage method produces natural killer cells that comprise at least 20% CD56+CD3− natural killer cells. In certain aspects, the three-stage method produces natural killer cells that comprise at least 40% CD56+CD3− natural killer cells. In certain aspects, the three-stage method produces natural killer cells that comprise at least 60% CD56+CD3− natural killer cells. In certain aspects, the three-stage method produces natural killer cells that comprise at least 70% CD56+CD3− natural killer cells. In certain aspects, the three-stage method produces natural killer cells that comprise at least 80% CD56+CD3− natural killer cells.

In certain aspects, the three-stage method disclosed herein produces natural killer cells that comprise at least 20% CD56+CD3−CD11a+ natural killer cells. In certain aspects, the three-stage method disclosed herein produces natural killer cells that comprise at least 40% CD56+CD3−CD11a+ natural killer cells. In certain aspects, the three-stage method disclosed herein produces natural killer cells that comprise at least 60% CD56+CD3−CD11a+ natural killer cells. In certain aspects, the three-stage method disclosed herein produces natural killer cells that comprise at least 80% CD56+CD3−CD11a+ natural killer cells.

In certain aspects, the three-stage method disclosed herein produces ILC3 cells that comprise at least 20% CD56+CD3− CD11a− ILC3 cells. In certain aspects, the three-stage method disclosed herein produces ILC3 cells that comprise at least 40% CD56+CD3−CD11a− ILC3 cells. In certain aspects, the three-stage method disclosed herein produces ILC3 cells that comprise at least 60% CD56+CD3− CD11a− ILC3 cells. In certain aspects, the three-stage method disclosed herein produces natural killer cells that comprise at least 80% CD56+CD3− CD11a− ILC3 cells.

In certain aspects, the three-stage method produces natural killer cells that exhibit at least 20% cytotoxicity against K562 cells when said natural killer cells and said K562 cells are co-cultured in vitro or ex vivo at a ratio of 10:1. In certain aspects, the three-stage method produces natural killer cells that exhibit at least 35% cytotoxicity against the K562 cells when said natural killer cells and said K562 cells are co-cultured in vitro or ex vivo at a ratio of 10:1. In certain aspects, the three-stage method produces natural killer cells that exhibit at least 45% cytotoxicity against the K562 cells when said natural killer cells and said K562 cells are co-cultured in vitro or ex vivo at a ratio of 10:1. In certain aspects, the three-stage method produces natural killer cells that exhibit at least 60% cytotoxicity against the K562 cells when said natural killer cells and said K562 cells are co-cultured in vitro or ex vivo at a ratio of 10:1. In certain aspects, the three-stage method produces natural killer cells that exhibit at least 75% cytotoxicity against the K562 cells when said natural killer cells and said K562 cells are co-cultured in vitro or ex vivo at a ratio of 10:1.

In certain aspects, the three-stage method produces ILC3 cells that exhibit at least 20% cytotoxicity against K562 cells when said ILC3 cells and said K562 cells are co-cultured in vitro or ex vivo at a ratio of 10:1. In certain aspects, the three-stage method produces ILC3 cells that exhibit at least 35% cytotoxicity against the K562 cells when said ILC3 cells and said K562 cells are co-cultured in vitro or ex vivo at a ratio of 10:1. In certain aspects, the three-stage method produces ILC3 cells that exhibit at least 45% cytotoxicity against the K562 cells when said ILC3 cells and said K562 cells are co-cultured in vitro or ex vivo at a ratio of 10:1. In certain aspects, the three-stage method produces ILC3 cells that exhibit at least 60% cytotoxicity against the K562 cells when said ILC3 cells and said K562 cells are co-cultured in vitro or ex vivo at a ratio of 10:1. In certain aspects, the three-stage method produces ILC3 cells that exhibit at least 75% cytotoxicity against the K562 cells when said ILC3 cells and said K562 cells are co-cultured in vitro or ex vivo at a ratio of 10:1.

In certain aspects, after said third culturing step, said third population of cells, e.g., said population of natural killer cells and/or ILC3 cells, is cryopreserved. In certain aspects, after said fourth step, said fourth population of cells, e.g., said population of natural killer cells and/or ILC3 cells, is cryopreserved.

In certain aspects, provided herein are populations of cells comprising natural killer cells, i.e., natural killers cells produced by a three-stage method described herein. Accordingly, provided herein is an isolated natural killer cell population produced by a three-stage method described herein. In a specific embodiment, said natural killer cell population comprises at least 20% CD56+CD3− natural killer cells. In a specific embodiment, said natural killer cell population comprises at least 40% CD56+CD3− natural killer cells. In a specific embodiment, said natural killer cell population comprises at least 60% CD56+CD3− natural killer cells. In a specific embodiment, said natural killer cell population comprises at least 80% CD56+CD3− natural killer cells. In a specific embodiment, said natural killer cell population comprises at least 60% CD16− cells. In a specific embodiment, said natural killer cell population comprises at least 80% CD16− cells. In a specific embodiment, said natural killer cell population comprises at least 20% CD94+ cells. In a specific embodiment, said natural killer cell population comprises at least 40% CD94+ cells.

In certain aspects, provided herein is a population of natural killer cells that is CD56+CD3− CD117+CD11a+, wherein said natural killer cells express perforin and/or EOMES, and do not express one or more of RORγt, aryl hydrocarbon receptor (AHR), and IL1R1. In certain aspects, said natural killer cells express perforin and EOMES, and do not express any of RORγt, aryl hydrocarbon receptor, or IL1R1. In certain aspects, said natural killer cells additionally express T-bet, GZMB, NKp46, NKp30, and NKG2D. In certain aspects, said natural killer cells express CD94. In certain aspects, said natural killer cells do not express CD94.

In certain aspects, provided herein is a population of ILC3 cells that is CD56+CD3− CD117+CD11a−, wherein said ILC3 cells express one or more of RORγt, aryl hydrocarbon receptor, and IL1R1, and do not express one or more of CD94, perforin, and EOMES. In certain aspects, said ILC3 cells express RORγt, aryl hydrocarbon receptor, and IL1R1, and do not express any of CD94, perforin, or EOMES. In certain aspects, said ILC3 cells additionally express CD226 and/or 2B4. In certain aspects, said ILC3 cells additionally express one or more of IL-22, TNFα, and DNAM-1. In certain aspects, said ILC3 cells express CD226, 2B4, IL-22, TNFα, and DNAM-1.

In certain aspects, provided herein is a method of producing a cell population comprising natural killer cells and ILC3 cells, comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; (c) culturing the second population of cells in a third medium comprising IL-2 and IL-15, and lacking each of a stem cell mobilizing agent and LMWH, to produce a third population of cells; and (d) separating CD11a+ cells and CD11a− cells from the third population of cells; and (e) combining the CD11a+ cells with the CD11a− cells in a ratio of 50:1, 40:1, 30:1, 20:1, 10:1, 5:1, 4:1, 3:1, 2:1, 1:1, 1:2, 1:3, 1:4, 1:5, 1:10, 1:20, 1:30, 1:40, or 1:50 to produce a fourth population of cells. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin. In certain aspects, in the fourth population of cells, the CD11a+ cells and CD11a− cells are combined in a ratio of 50:1. In certain aspects, in the fourth population of cells, the CD11a+ cells and CD11a− cells are combined in a ratio of 20:1. In certain aspects, in the fourth population of cells, the CD11a+ cells and CD11a− cells are combined in a ratio of 10:1. In certain aspects, in the fourth population of cells, the CD11a+ cells and CD11a− cells are combined in a ratio of 5:1. In certain aspects, in the fourth population of cells, the CD11a+ cells and CD11a− cells are combined in a ratio of 1:1. In certain aspects, in the fourth population of cells, the CD11a+ cells and CD11a− cells are combined in a ratio of 1:5. In certain aspects, in the fourth population of cells, the CD11a+ cells and CD11a− cells are combined in a ratio of 1:10. In certain aspects, in the fourth population of cells, the CD11a+ cells and CD11a− cells are combined in a ratio of 1:20. In certain aspects, in the fourth population of cells, the CD11a+ cells and CD11a− cells are combined in a ratio of 1:50.

5.3. Stem Cell Mobilizing Factors

5.3.1. Chemistry Definitions

To facilitate understanding of the disclosure of stem cell mobilizing factors set forth herein, a number of terms are defined below.

Generally, the nomenclature used herein and the laboratory procedures in biology, cellular biology, biochemistry, organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

As used herein, any “R” group(s) such as, without limitation, R^{a}, R^{b}, R^{c}, R^{d}, R^{e}, R^{f}, R^{g}, R^{h}, R^{m}, R^{G}, R^{J}, R^{K}, R^{U}, R^{V}, R^{Y}, and R^{Z} represent substituents that can be attached to the indicated atom. An R group may be substituted or unsubstituted. If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

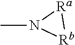

In addition, if two “R” groups are described as being “taken together” with the atom(s) to which they are attached to form a ring as an alternative, the R groups are not limited to the variables or substituents defined previously.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, acylalkyl, hydroxy, alkoxy, alkoxyalkyl, aminoalkyl, amino acid, aryl, heteroaryl, heterocyclyl, aryl(alkyl), heteroaryl(alkyl), heterocyclyl(alkyl), hydroxyalkyl, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, azido, nitro, silyl, sulfenyl, sulfinyl, sulfonyl, haloalkyl, haloalkoxy, trihalomethanesulfonyl, trihalomethanesulfonamido, an amino, a mono-substituted amino group and a di-substituted amino group.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, aryl, heteroaryl or heteroalicyclyl group. That is, the alkyl, alkenyl, alkynyl, ring(s) of the cycloalkyl, ring(s) of the cycloalkenyl, ring(s) of the aryl, ring(s) of the heteroaryl or ring(s) of the heteroalicyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, alkenyl, alkynyl, cycloalkyl cycloalkenyl, aryl, heteroaryl or heteroalicyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, “alkenyl” refers to an alkyl group that contains in the straight or branched hydrocarbon chain one or more double bonds. Examples of alkenyl groups include allenyl, vinylmethyl and ethenyl. An alkenyl group may be unsubstituted or substituted.

As used herein, “alkynyl” refers to an alkyl group that contains in the straight or branched hydrocarbon chain one or more triple bonds. Examples of alkynyls include ethynyl and propynyl. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s) or 3 to 8 atoms in the ring(s). When composed of two or more rings, the rings may be connected together in a fused fashion. A cycloalkenyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic or multicyclic aromatic ring system (a ring system with fully delocalized pi-electron system) that contain(s) one, two, three or more heteroatoms, that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, those described herein and the following: furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic, and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur, and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heterocyclyl may be quaternized. Heterocyclyl or heteroalicyclic groups may be unsubstituted or substituted. Examples of such “heterocyclyl” or “heteroalicyclyl” groups include, but are not limited to, those described herein and the following: 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 1,3-thiazinane, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline, and 3,4-methylenedioxyphenyl).

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, 2-phenylalkyl, 3-phenylalkyl and naphthylalkyl.

As used herein, “heteroaralkyl” and “heteroaryl(alkyl)” refer to a heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl, imidazolylalkyl and their benzo-fused analogs.

A “heteroalicyclyl(alkyl)” and “heterocyclyl(alkyl)” refer to a heterocyclic or a heteroalicyclylic group connected, as a substituent, via a lower alkylene group. The lower alkylene and heterocyclyl of a heteroalicyclyl(alkyl) may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl), and 1,3-thiazinan-4-yl(methyl).

“Lower alkylene groups” are straight-chained —CH₂— tethering groups, forming bonds to connect molecular fragments via their terminal carbon atoms. Examples include but are not limited to methylene (—CH₂—), ethylene (—CH₂CH₂—), propylene (—CH₂CH₂CH₂—), and butylene (—CH₂CH₂CH₂CH₂—). A lower alkylene group can be substituted by replacing one or more hydrogen of the lower alkylene group with a substituent(s) listed under the definition of “substituted.”

As used herein, “alkoxy” refers to the formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, “acyl” refers to a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) connected, as substituents, via a carbonyl group. Examples include formyl, acetyl, propanoyl, benzoyl and acryl. An acyl may be substituted or unsubstituted.

As used herein, “acylalkyl” refers to an acyl connected, as a substituent, via a lower alkylene group. Examples include aryl-C(═O)—(CH₂)ₙ— and heteroaryl-C(═O)—(CH₂)ₙ—, where n is an integer in the range of 1 to 6.

As used herein, “alkoxyalkyl” refers to an alkoxy group connected, as a substituent, via a lower alkylene group. Examples include C₁₋₄ alkyl-O—(CH₂)ₙ—, wherein n is an integer in the range of 1 to 6.

As used herein, “aminoalkyl” refers to an optionally substituted amino group connected, as a substituent, via a lower alkylene group. Examples include H₂N(CH₂)ₙ—, wherein n is an integer in the range of 1 to 6.

As used herein, “hydroxyalkyl” refers to an alkyl group in which one or more of the hydrogen atoms are replaced by a hydroxy group. Exemplary hydroxyalkyl groups include but are not limited to, 2-hydroxyethyl, 3-hydroxypropyl, 2-hydroxypropyl, and 2,2-dihydroxyethyl. A hydroxyalkyl may be substituted or unsubstituted.

As used herein, “haloalkyl” refers to an alkyl group in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl, chloro-fluoroalkyl, chloro-difluoroalkyl and 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, chloro-fluoroalkyl, chloro-difluoroalkoxy and 2-fluoroisobutoxy. A haloalkoxy may be substituted or unsubstituted.

A “sulfenyl” group refers to an “—SR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

An “O-carboxy” group refers to a “RC(═O)O—” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CSO₂—” group wherein each X is a halogen.

A “trihalomethanesulfonamido” group refers to an “X₃CS(O)₂N(R_{A})—” group wherein each X is a halogen, and R_{A} hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl).

The term “amino” as used herein refers to a —NH₂ group.

As used herein, the term “hydroxy” refers to a —OH group.

A “cyano” group refers to a “—CN” group.

The term “azido” as used herein refers to a —N3 group.

An “isocyanato” group refers to a “—NCO” group.

A “thiocyanato” group refers to a “—CNS” group.

An “isothiocyanato” group refers to an “—NCS” group.

A “carbonyl” group refers to a C═O group.

An “S-sulfonamido” group refers to a “—SO₂N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An S-sulfonamido may be substituted or unsubstituted.

An “N-sulfonamide” group refers to a “RSO₂N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-sulfonamido may be substituted or unsubstituted.

An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-carbamyl may be substituted or unsubstituted.

An “N-carbamyl” group refers to an “ROC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

An “O-thiocarbamyl” group refers to a “—OC(═S)—N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A C-amido may be substituted or unsubstituted.

An “N-amido” group refers to a “RC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-amido may be substituted or unsubstituted.

A “urea” group refers to “N(R)—C(═O)—NR_{A}R_{B} group in which R can be hydrogen or an alkyl, and R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A urea may be substituted or unsubstituted.

The term “halogen atom” or “halogen” as used herein, means any one of the radio-stable atoms of column 7 of the Periodic Table of the Elements, such as, fluorine, chlorine, bromine and iodine.

As used herein, “ [symbol] ” indicates a single or double bond, unless stated otherwise.

Where the numbers of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

In certain embodiments, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of the desired enantiomer and about 5% or less of the less preferred enantiomer based on the total weight of the two enantiomers in question.

In describing an optically active compound, the prefixes R and S are used to denote the absolute configuration of the optically active compound about its chiral center(s). The (+) and (−) are used to denote the optical rotation of an optically active compound, that is, the direction in which a plane of polarized light is rotated by the optically active compound. The (−) prefix indicates that an optically active compound is levorotatory, that is, the compound rotates the plane of polarized light to the left or counterclockwise. The (+) prefix indicates that an optically active compound is dextrorotatory, that is, the compound rotates the plane of polarized light to the right or clockwise. However, the sign of optical rotation, (+) and (−), is not related to the absolute configuration of a compound, R and S.

The term “isotopic variant” refers to a compound that contains an unnatural proportion of an isotope at one or more of the atoms that constitute such a compound. In certain embodiments, an “isotopic variant” of a compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N) nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O) fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I) iodine-125 (¹²⁵I) iodine-127 (¹²⁷I), iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an “isotopic variant” of a compound is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I) iodine-125 (¹²⁵I) iodine-129 (¹²⁹I) and iodine-131 (¹³¹I). It will be understood that, in a compound as provided herein, any hydrogen can be ²H, for example, or any carbon can be ¹³C, for example, or any nitrogen can be ¹⁵N, for example, or any oxygen can be ¹⁸O, for example, where feasible according to the judgment of one of skill. In certain embodiments, an “isotopic variant” of a compound contains unnatural proportions of deuterium (D).

The term “solvate” refers to a complex or aggregate formed by one or more molecules of a solute, e.g., a compound provided herein, and one or more molecules of a solvent, which present in a stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The phrase “an enantiomer, a mixture of enantiomers, a mixture of two or more diastereomers, or an isotopic variant thereof; or a pharmaceutically acceptable salt, solvate, hydrate, or prodrug thereof” has the same meaning as the phrase “(i) an enantiomer, a mixture of enantiomers, a mixture of two or more diastereomers, or an isotopic variant of the compound referenced therein; (ii) a pharmaceutically acceptable salt, solvate, hydrate, or prodrug of the compound referenced therein; or (iii) a pharmaceutically acceptable salt, solvate, hydrate, or prodrug of an enantiomer, a mixture of enantiomers, a mixture of two or more diastereomers, or an isotopic variant of the compound referenced therein.”

5.3.2. Stem Cell Mobilizing Compounds

In certain aspects, the stem cell mobilizing factor is a compound having Formula (I), (I-A), (I-B), (I-C), or (I-D), as described below.

### Formula (I)

Some embodiments disclosed herein relate to a compound of Formula (I), or a pharmaceutically acceptable salt thereof, having the structure:

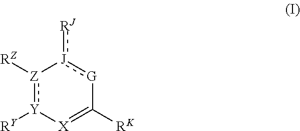

wherein: each [symbol] can independently represent a single bond or a double bond; R^{J} can be selected from the group consisting of —NR^{a}R^{b}, —OR^{b}, and ═O; wherein if R^{J} is ═O, then [symbol] joining G and J represents a single bond and G is N and the N is substituted with R^{G}; otherwise [symbol] joining G and J represents a double bond and G is N; R^{a} can be hydrogen or C₁-C₄ alkyl; R^{b} can be R^{c} or —(C₁-C₄ alkyl)-R^{c}; R^{c} can be selected from the group consisting of: —OH, —O(C₁-C₄ alkyl), —O(C₁-C₄ haloalkyl); —C(═O)NH₂; unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted can be substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: hydrogen, unsubstituted C₁₋₆ alkyl; substituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted can be substituted with one or more substituents Q, wherein each Q is independently selected from the group consisting of: —OH, C₁₋₄ alkyl, C₁₋₄ haloalkyl, halo, cyano, —O—(C₁₋₄ alkyl), and —O—(C₁₋₄ haloalkyl); R^{G} can be selected from the group consisting of hydrogen, C₁₋₄ alkyl, and —(C₁₋₄ alkyl)-C(═O)NH₂; R^{Y} and R^{Z} can each independently be absent or be selected from the group consisting of: hydrogen, halo, C₁₋₆ alkyl, —OH, —O—(C₁₋₄ alkyl), —NH(C₁₋₄ alkyl), and —N(C₁₋₄ alkyl)₂; or R^{Y} and R^{Z} taken together with the atoms to which they are attached can joined together to form a ring selected from:

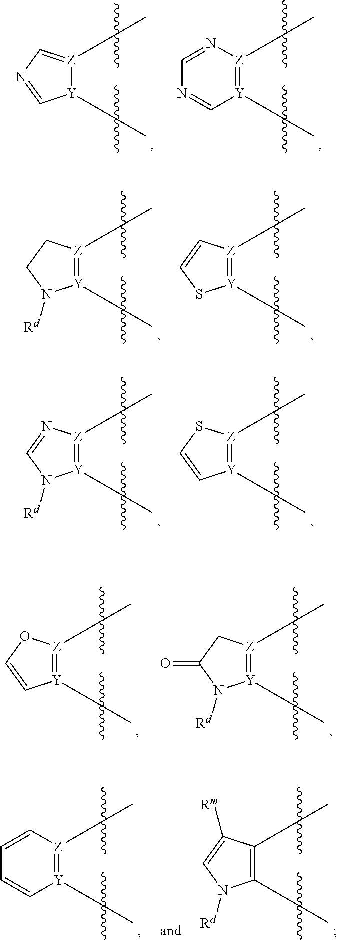

wherein said ring can be optionally substituted with one, two, or three groups independently selected from C₁₋₄ alkyl, C₁₋₄ haloalkyl, halo, cyano, —OH, —O—(C₁₋₄ alkyl), —N(C₁₋₄ alkyl)₂, unsubstituted C₆-C₁₀ aryl, C₆-C₁₀ aryl substituted with 1-5 halo atoms, and —O—(C₁₋₄ haloalkyl); and wherein if R^{Y} and R^{Z} taken together forms

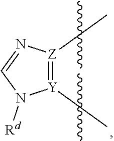

then R^{J} can be —OR^{b} or ═O; R^{d} can be hydrogen or C₁-C₄ alkyl; R^{m} can be selected from the group consisting of C₁₋₄ alkyl, halo, and cyano; J can be C; and X, Y, and Z can each be independently N or C, wherein the valency of any carbon atom is filled as needed with hydrogen atoms.

In some embodiments, [symbol] can represent a single bond. In other embodiments, [symbol] can represent a double bond. In some embodiments, [symbol] joining Y and Z can represent a single bond. In other embodiments, [symbol] joining Y and Z can represent a double bond. In some embodiments, when [symbol] joining G and J represents a single bond, G can be N and the N is substituted with R^{G}. In other embodiments, when [symbol] joining G and J represents a double bond, G can be N. In some embodiments, when [symbol] joining G and J represents a double bond, then [symbol] joining J and R^{J} can be a single bond. In some embodiments, when [symbol] joining G and J represents a double bond, then [symbol] joining J and R^{J} can not be a double bond. In some embodiments, when [symbol] joining J and R^{J} represents a double bond, then [symbol] joining G and J can be a single bond. In some embodiments, when [symbol] joining J and R^{J} represents a double bond, then joining G and J can not be a double bond.

In some embodiments, R^{J} can be —NR^{a}R^{b}. In other embodiments, R^{J} can be —OR^{b}. In still other embodiments, R^{J} can be ═O. In some embodiments, when R^{J} is ═O, then [symbol] joining G and J represents a single bond and G is N and the N is substituted with R^{G}. In some embodiments, R^{G} is —CH₂CH₂—C(═O)NH₂.

In some embodiments, R^{a} can be hydrogen. In some embodiments, R^{a} can be C₁-C₄ alkyl. For example, R^{a} can be methyl, ethyl, n-propyl, iso-propyl, n-butyl, iso-butyl or tert-butyl.

In some embodiments, R^{b} can be R^{c}. In some embodiments, R^{b} can be —(C₁-C₄ alkyl)-R^{c}. For example, R^{b} can be —CH₂—R^{c}, —CH₂CH₂—R^{c}, —CH₂CH₂CH₂—R^{c}, or —CH₂CH₂CH₂CH₂—R^{c}. In some embodiments, when R^{b} is —CH₂CH₂—R^{c}, R^{c} can be —O(C₁-C₄ alkyl). In other embodiments, when R^{b} is —CH₂CH₂—R^{c}, R^{c} can be —O(C₁-C₄ haloalkyl). In still other embodiments, when R^{b} is —CH₂CH₂—R^{c}, R^{c} can be —C(═O)NH₂.

In some embodiments, R^{c} can be —OH. In some embodiments, R^{c} can be —O(C₁-C₄ alkyl). In some embodiments, R^{c} can be —O(C₁-C₄ haloalkyl). In some embodiments, R^{c} can be —C(═O)NH₂. In some embodiments, R^{c} can be unsubstituted C₆₋₁₀ aryl. In some embodiments, R^{c} can be substituted C₆₋₁₀ aryl. In some embodiments, R^{c} can be unsubstituted five- to ten-membered heteroaryl having 1-4 atoms selected from the group consisting of O, N, and S. In some embodiments, R^{c} can be substituted five- to ten-membered heteroaryl having 1-4 atoms selected from the group consisting of O, N, and S. In some embodiments, when a R^{c} moiety is indicated as substituted, the moiety can be substituted with one or more, for example, one, two, three, or four substituents E. In some embodiments, E can be —OH. In some embodiments, E can be C₁-C₄ alkyl. In some embodiments, E can be C₁-C₄ haloalkyl. In some embodiments, E can be —O(C₁-C₄ alkyl). In some embodiments, E can be —O(C₁-C₄ haloalkyl).

In some embodiments, when R^{b} is —CH₂CH₂—R^{c}, R^{c} can be unsubstituted C₆₋₁₀ aryl. In other embodiments, when R^{b} is —CH₂CH₂—R^{c}, R^{c} can be substituted C₆₋₁₀ aryl. In still other embodiments, when R^{b} is —CH₂CH₂—R^{c}, R^{c} can be unsubstituted five- to ten-membered heteroaryl having 1-4 atoms selected from the group consisting of O, N, and S. In yet still other embodiments, R^{b} can be —(C₁-C₄ alkyl)-R^{c} and R^{c} can be substituted five- to ten-membered heteroaryl having 1-4 atoms selected from the group consisting of O, N, and S. When a R^{c} moiety is indicated as substituted, the moiety can be substituted with one or more, for example, one, two, three, or four substituents E. In some embodiments, E can be —OH. In other embodiments, E can be C₁-C₄ alkyl. In still other embodiments, E can be C₁-C₄ haloalkyl. In still other embodiments, E can be —O(C₁-C₄ alkyl). In still other embodiments, E can be —O(C₁-C₄ haloalkyl).

In some embodiments, when R^{b} is —CH₂CH₂—R^{c}, R^{c} can be phenyl. In other embodiments, when R^{b} is —CH₂CH₂—R^{c}, R^{c} can be naphthyl. In still other embodiments, when R^{b} is —CH₂CH₂—R^{c}, R^{c} can be hydroxyphenyl. In still other embodiments, when R^{b} is —CH₂CH₂—R^{c}, R^{c} can be indolyl.

In some embodiments, R^{K} can be hydrogen. In other embodiments, R^{K} can be unsubstituted C₁₋₆ alkyl. For example, in some embodiments, R^{K} can be methyl, ethyl, n-propyl, iso-propyl, n-butyl, iso-butyl, tert-butyl, pentyl (branched and straight-chained), or hexyl (branched and straight-chained). In other embodiments, R^{K} can be substituted C₁₋₆ alkyl. In other embodiments, R^{K} can be —NH(C₁₋₄ alkyl). For example, in some embodiments, R^{K} can be —NH(CH₃), —NH(CH₂CH₃), —NH(isopropyl), or —NH(sec-butyl). In other embodiments, R^{K} can be —N(C₁₋₄ alkyl)₂.

In some embodiments, R^{K} can be unsubstituted C₆₋₁₀ aryl. In other embodiments, R^{K} can be substituted C₆₋₁₀ aryl. In other embodiments, R^{K} can be unsubstituted five- to ten-membered heteroaryl having 1-4 atoms selected from the group consisting of O, N, and S. In other embodiments, R^{K} can be substituted five- to ten-membered heteroaryl having 1-4 atoms selected from the group consisting of O, N, and S. When a R^{K} moiety is indicated as substituted, the moiety can be substituted with one or more, for example, one, two, three, or four substituents Q. In some embodiments, Q can be —OH. In other embodiments, Q can be C₁₋₄ alkyl. In still other embodiments, Q can be C₁₋₄ haloalkyl. In still other embodiments, Q can be halo. In still other embodiments, Q can be cyano. In still other embodiments, Q can be —O—(C₁₋₄ alkyl). In still other embodiments, Q can be —O—(C₁₋₄ haloalkyl).

In some embodiments, R^{K} can be phenyl or naphthyl. In other embodiments, R^{K} can be benzothiophenyl. In other embodiments, R^{K} can be benzothiophenyl. In other embodiments, R^{K} can be benzothiophenyl. In still other embodiments, R^{K} can be pyridinyl. In yet still other embodiments, R^{K} can be pyridinyl substituted with one or more substituents Q. For example, R^{K} can be methylpyridinyl, ethylpyridinyl cyanopyridinyl, chloropyridinyl, fluoropyridinyl, or bromopyridinyl.

In some embodiments, R^{G} can be hydrogen. In some embodiments, R^{G} can be C₁₋₄ alkyl. In some embodiments, R^{G} can be —(C₁₋₄ alkyl)-C(═O)NH₂.

In some embodiments, R^{Y} and R^{Z} can independently be absent. In other embodiments, R^{Y} and R^{Z} can independently be hydrogen. In other embodiments, R^{Y} and R^{Z} can independently be halo. In other embodiments, R^{Y} and R^{Z} can independently be C₁₋₆ alkyl. In other embodiments, R^{Y} and R^{Z} can independently be —OH. In still other embodiments, R^{Y} and R^{Z} can independently be —O—(C₁₋₄ alkyl). In other embodiments, R^{Y} and R^{Z} can independently be —NH(C₁₋₄ alkyl). For example, R^{Y} and R^{Z} can independently be —NH(CH₃), —NH(CH₂CH₃), —NH(isopropyl), or —NH(sec-butyl). In other embodiments, R^{Y} and R^{Z} can independently be —N(C₁₋₄ alkyl)₂.

In some embodiments, R^{Y} and R^{Z} taken together with the atoms to which they are attached can be joined together to form a ring. In some embodiments, R^{Y} and R^{Z} taken together with the atoms to which they are attached can be joined together to form

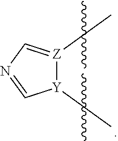

In other embodiments, R^{Y} and R^{Z} taken together with the atoms to which they are attached can be joined together to form

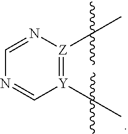

In other embodiments, R^{Y} and R^{Z} taken together with the atoms to which they are attached can be joined together to form

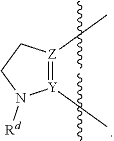

In still other embodiments, R^{Y} and R^{Z} taken together with the atoms to which they are attached can be joined together to form

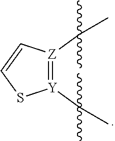

In yet still other embodiments, R^{Y} and R^{Z} taken together with the atoms to which they are attached can be joined together to form

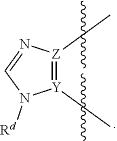

In other embodiments, R^{Y} and R^{Z} taken together with the atoms to which they are attached can be joined together to form

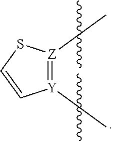

In yet other embodiments, R^{Y} and R^{Z} taken together with the atoms to which they are attached can be joined together to form

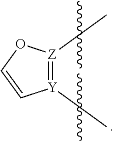

In yet still other embodiments, R^{Y} and R^{Z} taken together with the atoms to which they are attached can be joined together to form

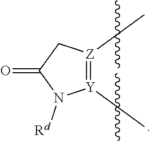

In other embodiments, R^{Y} and R^{Z} taken together with the atoms to which they are attached can be joined together to form

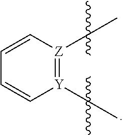

In still other embodiments, R^{Y} and R^{Z} taken together with the atoms to which they are attached can be joined together to form and

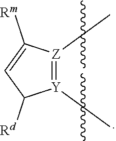

In some embodiments, when R^{Y} and R^{Z} taken together with the atoms to which they are attached can be joined together to form a ring, the ring can be substituted with one, two, or three groups independently selected from C₁-C₄ alkyl, —N(C₁-C₄ alkyl)₂, cyano, unsubstituted phenyl, and phenyl substituted with 1-5 halo atoms.

In some embodiments, when R^{Y} and R^{Z} taken together forms

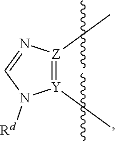

then R^{J} can be —OR^{b} or ═O.

In some embodiments, R^{Y} and R^{Z} taken together with the atoms to which they are attached can be joined together to form

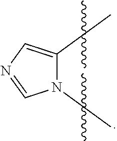 [SMILES: *c1cncn1*]

In other embodiments, R^{Y} and R^{Z} taken together with the atoms to which they are attached can be joined together to form

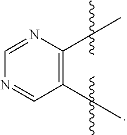 [SMILES: *c1cncnc1*]

In other embodiments, R^{Y} and R^{Z} taken together with the atoms to which they are attached can be joined together to form

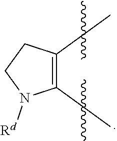

In other embodiments, R^{Y} and R^{Z} taken together with the atoms to which they are attached can be joined together to form

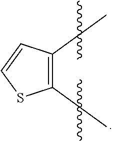 [SMILES: *c1ccsc1*]

In other embodiments, R^{Y} and R^{Z} taken together with the atoms to which they are attached can be joined together to form

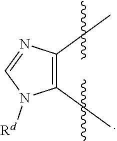

In other embodiments, R^{Y} and R^{Z} taken together with the atoms to which they are attached can be joined together to form

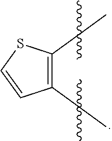 [SMILES: *c1ccsc1*]

In other embodiments, R^{Y} and R^{Z} taken together with the atoms to which they are attached can be joined together to form

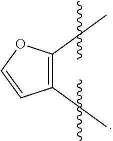 [SMILES: *c1ccoc1*]

In other embodiments, R^{Y} and R^{Z} taken together with the atoms to which they are attached can be joined together to form

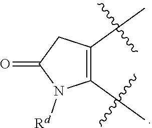

In other embodiments, R^{Y} and R^{Z} taken together with the atoms to which they are attached can be joined together to form

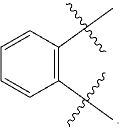 [SMILES: *c1ccccc1*]

In other embodiments, R^{Y} and R^{Z} taken together with the atoms to which they are attached can be joined together to form

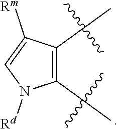

In some embodiments, when R^{Y} and R^{Z} taken together with the atoms to which they are attached can be joined together to form a ring, the ring can be substituted with one, two, or three groups independently selected from C₁-C₄ alkyl, —N(C₁-C₄ alkyl)₂, cyano, unsubstituted phenyl, and phenyl substituted with 1-5 halo atoms. In some embodiments, R^{Y} and R^{Z} taken together with the atoms to which they are attached can be

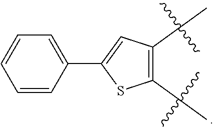 [SMILES: *c1cc(-c2ccccc2)sc1*]

In other embodiments, R^{Y} and R^{Z} taken together with the atoms to which they are attached can be

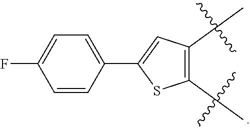 [SMILES: *c1cc(-c2ccc(F)cc2)sc1*]

In still other embodiments, R^{Y} and R^{Z} taken together with the atoms to which they are attached can be

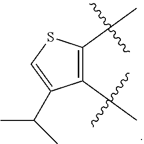 [SMILES: *c1scc(C(C)C)c1*]

In yet still other embodiments, R^{Y} and R^{Z} taken together with the atoms to which they are attached can be

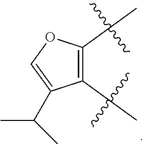 [SMILES: *c1occ(C(C)C)c1*]

In other embodiments, R^{Y} and R^{Z} taken together with the atoms to which they are attached can be

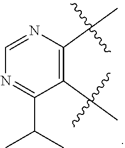 [SMILES: *c1ncnc(C(C)C)c1*]

In some embodiments, R^{d} can be hydrogen. In other embodiments, R^{d} can be C₁-C₄ alkyl. For example R^{d} can be methyl, ethyl, n-propyl, iso-propyl, n-butyl, iso-butyl or tert-butyl. In still other embodiments, R^{d} can be halo. In other embodiments, R^{d} can be cyano.

In some embodiments, R^{m} can be hydrogen. In other embodiments, R^{m} can be C₁-C₄ alkyl. For example R^{m} can be methyl, ethyl, n-propyl, iso-propyl, n-butyl, iso-butyl or tert-butyl. In still other embodiments, R^{m} can be halo. For example, R^{m} can be fluoro, chloro, bromo, or iodo. In other embodiments, R^{m} can be cyano.

In some embodiments, X, Y, and Z can each be independently N or C, wherein the valency of any carbon atom is filled as needed with hydrogen atoms. In some embodiments, X can be N, Y can be N, and Z can be N. In other embodiments, X can be N, Y can be N, and Z can be CH. In some embodiments, X can be N, Y can be CH, and Z can be N. In still other embodiments, X can be CH, Y can be N, and Z can be N. In yet still other embodiments, X can be CH, Y can be CH, and Z can be N. In other embodiments, X can be CH, Y can be N, and Z can be CH. In yet other embodiments, X can be N, Y can be CH, and Z can be CH. In other embodiments, X can be CH, Y can be CH, and Z can be CH.

In some embodiments, R^{a} can be hydrogen; R^{b} can be —(C₁-C₄ alkyl)-R^{c}; R^{c} can be selected from the group consisting of: —C(═O)NH₂; unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: hydrogen, unsubstituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); R^{G} can be —(C₁₋₄ alkyl)-C(═O)NH₂; R^{Y} and R^{Z} can each be independently absent or be selected from the group consisting of: hydrogen, C₁₋₆ alkyl, and —NH(C₁₋₄ alkyl); or R^{Y} and R^{Z} taken together with the atoms to which they are attached can be joined together to form a ring selected from:

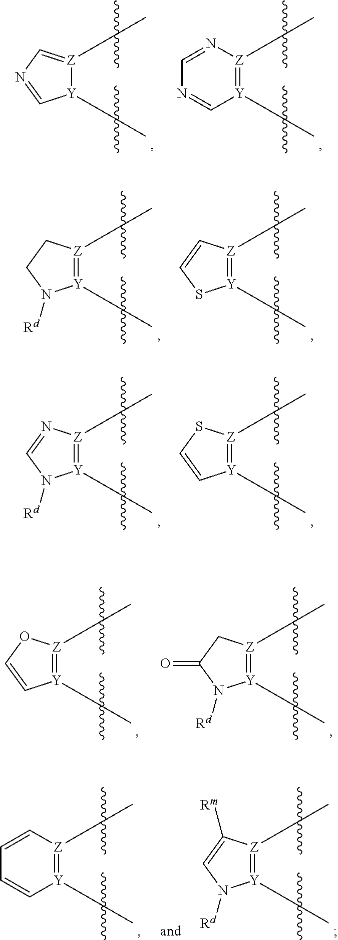

wherein said ring can be optionally substituted with one, two, or three groups independently selected from C₁₋₄ alkyl, C₁₋₄ haloalkyl, halo, cyano, —OH, —O—(C₁₋₄ alkyl), —N(C₁₋₄ alkyl)₂, unsubstituted C₆-C₁₀ aryl, C₆-C₁₀ aryl substituted with 1-5 halo atoms, and —O—(C₁₋₄ haloalkyl); R^{d} can be C₁-C₄ alkyl; R^{m} can be cyano; and X, Y, and Z can each be independently N or C, wherein the valency of any carbon atom is filled as needed with hydrogen atoms.

In some embodiments, R^{a} can be hydrogen; R^{b} can be —CH₂CH₂—R^{c}; R^{c} can be selected from the group consisting of: unsubstituted phenyl, substituted phenyl, indolyl, and —C(═O)NH₂; R^{K} can be selected from the group consisting of: hydrogen, methyl, substituted pyridinyl, unsubstituted benzothiophenyl, and —NH(C₁-C₄ alkyl); R^{G} can be —CH₂CH₂—C(═O)NH₂; R^{Y} can be —NH(C₁-C₄ alkyl); R^{Z} can be absent or hydrogen; or R^{Y} and R^{Z} taken together with the atoms to which they are attached can be joined together to form a ring selected from:

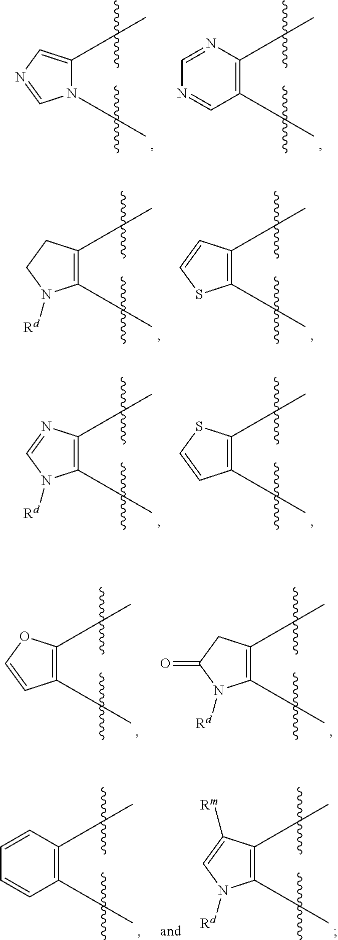

wherein said ring can be optionally substituted with one, two, or three groups independently selected from C₁-C₄ alkyl, —N(C₁-C₄ alkyl)₂, cyano, unsubstituted phenyl, and phenyl substituted with 1-5 halo atoms; R^{d} can be C₁-C₄ alkyl; R^{m} can be cyano; and X can be N or CH.

In some embodiments, when R^{J} is —NR^{a}R^{b}; G can be N; [symbol] joining G and J can be a double bond; R^{a} can hydrogen; R^{b} can be —CH₂CH₂—R^{c}; R^{c} can be unsubstituted five- to ten-membered heteroaryl having 1-4 atoms selected from the group consisting of O, N, and S; or R^{c} can be substituted C₆₋₁₀ aryl, substituted with one or more E, wherein E is —OH; R^{K} can be unsubstituted five- to ten-membered heteroaryl having 1-4 atoms selected from the group consisting of O, N, and S; or R^{K} can be substituted five- to ten-membered heteroaryl having 1-4 atoms selected from the group consisting of O, N, and S; substituted with one or more Q, wherein Q can be selected from cyano, halo, or C₁-C₄ alkyl; R^{Y} and R^{Z} taken together can be

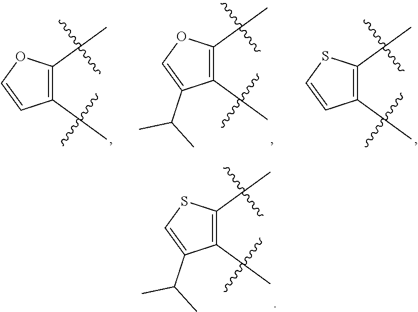

In some embodiments, when R^{J} is —NR^{a}R^{b}; G can be N; [symbol] joining G and J can be a double bond; R^{a} can hydrogen; R^{b} can be —CH₂CH₂—R^{c}; R^{c} can be unsubstituted five- to ten-membered heteroaryl having 1-4 atoms selected from the group consisting of O, N, and S; or R^{c} can be substituted C₆₋₁₀ aryl, substituted with one or more E, wherein E is —OH; R^{K} can be hydrogen, C₁₋₄ alkyl, or unsubstituted five- to ten-membered heteroaryl having 1-4 atoms selected from the group consisting of O, N, and S; and R^{Y} and R^{Z} taken together can be

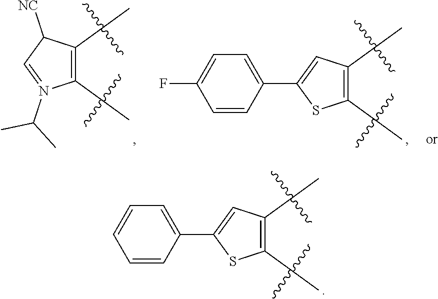

In some embodiments, when R^{J} is —NR^{a}R^{b}; G can be N; [symbol] joining G and J can be a double bond; R^{a} can hydrogen; R^{b} can be —CH₂CH₂—R^{c}; R^{c} can be unsubstituted five- to ten-membered heteroaryl having 1-4 atoms selected from the group consisting of O, N, and S; or R^{c} can be substituted C₆₋₁₀ aryl, substituted with one or more E, wherein E is —OH; R^{K} can be hydrogen, C₁₋₄ alkyl, or unsubstituted five- to ten-membered heteroaryl having 1-4 atoms selected from the group consisting of O, N, and S; and R^{Y} and R^{Z} taken together can be

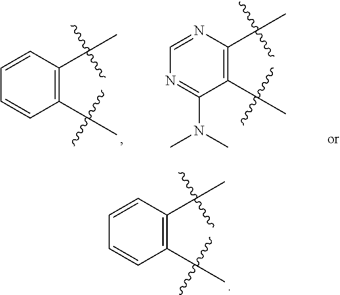 [SMILES: *c1ccccc1* ; *c1ncnc(N(C)C)c1* ; *c1ccccc1*]

In some embodiments, when R^{J} is —NR^{a}R^{b}; G can be N; [symbol] joining G and J can be a double bond, R^{a} can be hydrogen; R^{b} can be —CH₂CH₂—R^{c}; R^{c} can be substituted C₆₋₁₀ aryl; substituted with one or more E, wherein E can be —OH; R^{K} can be unsubstituted five- to ten-membered heteroaryl having 1-4 atoms selected from the group consisting of O, N, and S; R^{Y} can be —NH(C₁₋₄ alkyl); R^{Z} can be hydrogen; J can be C; X can be N; Y can be C; Z can be C; and [symbol] joining Y and Z can be a double bond. In some embodiments, the compound of Formula (I) can be 4-(2-((2-(benzo[b]thiophen-3-yl)-6-(isopropylamino)pyrimidin-4-yl)amino)ethyl)phenol.

In some embodiments, when R^{J} is —NR^{a}R^{b}; G can be N; [symbol] joining G and J can be a double bond; R^{a} can be hydrogen; R^{b} can be —CH₂CH₂—R^{c}, R^{c} can be substituted C₆₋₁₀-aryl, substituted with one or more E, wherein E can be —OH; R^{K} can be unsubstituted five- to ten-membered heteroaryl having 1-4 atoms selected from the group consisting of O, N, and S; R^{Y} and R^{Z} taken together is

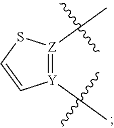

wherein the ring is substituted with C₁-C₄ alkyl; J can be C; X can be N; Y can be C; and Z can be C. In some embodiments, the compound of Formula (I) can be 4-(2-((2-(benzo[b]thiophen-3-yl)-7-isopropylthieno[3,2-d]pyrimidin-4-yl)amino)ethyl)phenol.

In some embodiments, when R^{J} is —NR^{a}R^{b}; G can be N; [symbol] joining G and J can be a double bond; R^{a} can be hydrogen; R^{b} can be —CH₂CH₂—R^{c}, R^{c} can be substituted C₆₋₁₀-aryl, substituted with one or more E, wherein E can be —OH; R^{K} can be unsubstituted five- to ten-membered heteroaryl having 1-4 atoms selected from the group consisting of O, N, and S; R^{Y} and R^{Z} taken together is

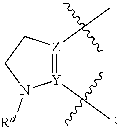

R^{d} can be C₁-C₄ alkyl; J can be C; X can be N; Y can be C; and Z can be C. In some embodiments, the compound of Formula (I) can be 4-(2-((2-(benzo[b]thiophen-3-yl)-7-isopropyl-6,7-dihydro-5H-pyrrolo[2,3-d]pyrimidin-4-yl)amino)ethyl)phenol.

In some embodiments, when R^{J} is —NR^{a}R^{b}; G can be N; [symbol] joining G and J can be a double bond; R^{a} can be hydrogen; R^{b} can be —CH₂CH₂—R^{c}, R^{c} can be substituted C₆₋₁₀-aryl, substituted with one or more E, wherein E can be —OH; R^{K} can be unsubstituted five- to ten-membered heteroaryl having 1-4 atoms selected from the group consisting of O, N, and S; R^{Y} and R^{Z} taken together is

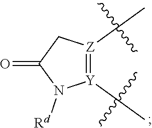

R^{d} can be C₁-C₄ alkyl; J can be C; X can be N; Y can be C; and Z can be C. In some embodiments, the compound of Formula (I) can be 2-(benzo[b]thiophen-3-yl)-4-(4-hydroxyphenethyl)amino)-7-isopropyl-5,7-dihydro-6H-pyrrolo[2,3-d]pyrimidin-6-one.

In some embodiments, when R^{J} is —OR^{J}; G can be N; [symbol] joining G and J can be a double bond; R^{b} can be —CH₂CH₂—R^{c}; R^{c} can be —C(═O)NH₂; R^{K} can unsubstituted five- to ten-membered heteroaryl having 1-4 atoms selected from the group consisting of O, N, and S; R^{Y} and R^{Z} taken together can be

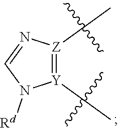

R^{d} can be C₁-C₄ alkyl; J can be C; X can be N; Y can be C; and Z is C. In some embodiments, the compound of Formula (I) can be 3-((2-(benzo[b]thiophen-3-yl)-9-isopropyl-9H-purin-6-yl)oxy)propanamide.

In some embodiments, when R^{J} is —NR^{a}R^{b}; G can be N; [symbol] joining G and J can be a double bond; R^{b} can be —CH₂CH₂—R^{c}; R^{c} can be substituted C₆₋₁₀ aryl, substituted with one or more E, wherein E is —OH; R^{K} is unsubstituted five- to ten-membered heteroaryl having 1-4 atoms selected from the group consisting of O, N, and S; R^{Y} and R^{Z} taken together can be

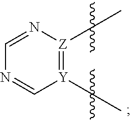

wherein said ring is substituted with —N(C₁₋₄ alkyl)₂; J can be C; X can be N; Y can be C; and Z is C. In some embodiments, the compound of Formula (I) can be 4-(2-((2-(benzo[b]thiophen-3-yl)-8-(dimethylamino)pyrimido[5,4-d]pyrimidin-4-yl)amino)ethyl)phenol.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is cyano; R^{Y} can be —NH(C₁₋₄ alkyl); R^{Z} can be absent; J can be C; X can be C; Y can be C; Z can be N; and [symbol] joining Y and Z can be a double bond. In some embodiments, the compound of Formula (I) can be 5-(2-((2-(1H-indol-3-yl)ethyl)amino)-6-(sec-butylamino)pyrimidin-4-yl)nicotinonitrile.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be unsubstituted C₁₋₆ alkyl; R^{Y} and R^{Z} taken together can

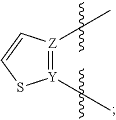

wherein the ring is substituted with unsubstituted C₆-C₁₀ aryl; J can be C; X can be N; Y can be C; Z can be C. In some embodiments, the compound of Formula (I) can be N-(2-(1H-indol-3-yl)ethyl)-2-methyl-6-phenylthieno[2,3-d]pyrimidin-4-amine

In some embodiments, when R^{J} can be —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be hydrogen; R^{Y} and R^{Z} taken together can be

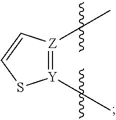

wherein the ring is substituted with substituted C₆-C₁₀ aryl; J can be C; X can be N; Y can be C; and Z can be C. In some embodiments, the compound of Formula (I) can be N-(2-(1H-indol-3-yl)ethyl)-6-(4-fluorophenyl)thieno[2,3-d]pyrimidin-4-amine

In some embodiments, when R^{J} is ═O; G can be N substituted with R^{G}; [symbol] joining G and J can be a single bond; R^{G} can be —(C₁₋₄ alkyl)-C(═O)NH₂; R^{K} can be unsubstituted five- to ten-membered heteroaryl having 1-4 atoms selected from the group consisting of O, N, and S; R^{Y} and R^{Z} taken together can be

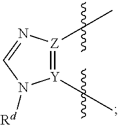

R^{d} can be C₁-C₄ alkyl; J can be C; X can be N; Y can be C; and Z can be C. In some embodiments, the compound of Formula (I) can be 3-(2-(benzo[b]thiophen-3-yl)-9-isopropyl-6-oxo-6,9-dihydro-1H-purin-1-yl)propanamide.

In some embodiments, when R^{J} is —NR^{a}R^{b}; G can be N; [symbol] joining G and J can be a double bond R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q can be halo; R^{Y} and R^{Z} taken together can be

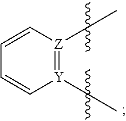

J can be C; X can be N; Y can be C; and Z can be C. In some embodiments, the compound of Formula (I) can be N-(2-(1H-indol-3-yl)ethyl)-2-(5-fluoropyridin-3-yl)quinazolin-4-amine.

In some embodiments, when R^{J} is —NR^{a}R^{b}; G is N; [symbol] joining G and J can be a double bond; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q can be cyano; R^{Y} and R^{Z} taken together is

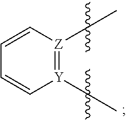

J can be C; X can be N; Y can be C; and Z can be C. In some embodiments, the compound of Formula (I) can be 5-(4-((2-(1H-indol-3-yl)ethyl)amino)quinazolin-2-yl)nicotinonitrile.

In some embodiments, when R^{J} is —NR^{a}R^{b}; G can be N; [symbol] joining G and J can be a double bond; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be —NH(C₁₋₄ alkyl); R^{Y} and R^{Z} taken together can be

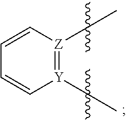

J can be C; X can be N; Y can be C; and Z can be C. In some embodiments, the compound of Formula (I) can be N⁴-(2-(1H-indol-3-yl)ethyl)-N²-(sec-butyl)quinazoline-2,4-diamine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be substituted C₆₋₁₀ aryl, substituted with one or more E, wherein E is —OH; R^{K} can be unsubstituted five- to ten-membered heteroaryl having 1-4 atoms selected from the group consisting of O, N, and S; R^{Y} and R^{Z} taken together can be

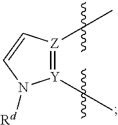

wherein the ring is substituted with cyano; R^{d} can be C₁-C₄ alkyl; J can be C; X can be N; Y can be C; and Z can be C. In some embodiments, the compound of Formula (I) can be 2-(benzo[b]thiophen-3-yl)-44(4-hydroxyphenethyl)amino)-7-isopropyl-7H-pyrrolo[2,3-d]pyrimidine-5-carbonitrile.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be unsubstituted five- to ten-membered heteroaryl having 1-4 atoms selected from the group consisting of O, N, and S; R^{Y} and R^{Z} taken together can be

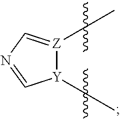

wherein the ring is substituted with C₁₋₄ alkyl; J can be C; X can be C; Y can be N; and Z can be C; wherein the valency of any carbon atom is filled as needed with hydrogen atoms. In some embodiments, the compound of Formula (I) can be N-(2-(1H-indol-3-yl)ethyl)-6-(benzo[b]thiophen-3-yl)-3-isopropylimidazo[1,5-c]pyrazin-8-amine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be substituted C₆₋₁₀-aryl, substituted with one or more E, wherein E is —OH; R^{K} can be unsubstituted five- to ten-membered heteroaryl having 1-4 atoms selected from the group consisting of O, N, and S; R^{Y} and R^{Z} taken together can be

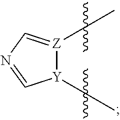

wherein the ring can be substituted with C₁₋₄ alkyl; J can be C; X can be C; Y can be N; and Z can be C; wherein the valency of any carbon atom is filled as needed with hydrogen atoms. In some embodiments, the compound of Formula (I) can be 4-(2-((6-(benzo[b]thiophen-3-yl)-3-isopropylimidazo[1,5-a]pyrazin-8-yl)amino)ethyl)phenol.

In some embodiments, when R^{J} is —NR^{a}R^{b}; G can be N; [symbol] joining G and J represents a double bond; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is cyano; R^{Y} and R^{Z} taken together is

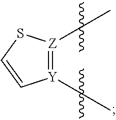

wherein the ring is substituted with C₁-C₄ alkyl; J can be C; X can be N; Y can be C; and Z can be C. In some embodiments, the compound of Formula (I) can be 5-(4-((2-(1H-indol-3-yl)ethyl)amino)-7-isopropylthieno[3,2-d]pyrimidin-2-yl)nicotinonitrile.

In some embodiments, when R^{J} is —NR^{a}R^{b}; G can be N; [symbol] joining G and J represents a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is halo; R^{Y} and R^{Z} taken together can be

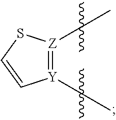

wherein the ring is substituted with C₁-C₄ alkyl; J can be C; X can be N; Y can be C; and Z can be C. In some embodiments, the compound of Formula (I) can be N-(2-(1H-indol-3-yl)ethyl)-2-(5-fluoropyridin-3-yl)-7-isopropylthieno[3,2-d]pyrimidin-4-amine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is halo; R^{Y} and R^{Z} taken together can be

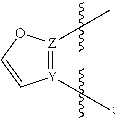

J can be C; X can be N; Y can be C; and Z can be C. In some embodiments, the compound of Formula (I) can be N-(2-(1H-indol-3-yl)ethyl)-2-(5-fluoropyridin-3-yl)furo[3,2-d]pyrimidin-4-amine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is C₁-C₄ alkyl; R^{Y} and R^{Z} taken together can be

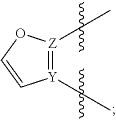

J can be C; X can be N; Y can be C; and Z can be C. In some embodiments, the compound of Formula (I) can be N-(2-(1H-indol-3-yl)ethyl)-2-(5-methylpyridin-3-yl)furo[3,2-d]pyrimidin-4-amine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is C₁-C₄ alkyl; R^{Y} and R^{Z} taken together can be

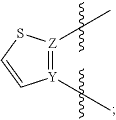

wherein the ring is substituted with C₁-C₄ alkyl J can be C; X can be N; Y can be C; and Z can be C. In some embodiments, the compound of Formula (I) can be N-(2-(1H-indol-3-yl)ethyl)-7-isopropyl-2-(5-methylpyridin-3-yl)thieno[3,2-d]pyrimidin-4-amine.

In some embodiments, when R^{J} is —NR^{a}R^{b}; G is N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is cyano; R^{Y} and R^{Z} taken together can be

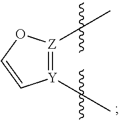

J can be C; X can be N; Y can be C; and Z can be C. In some embodiments, the compound of Formula (I) can be 5-(4-((2-(1H-indol-3-yl)ethyl)amino)furo[3,2-d]pyrimidin-2-yl)nicotinonitrile.

In some embodiments, provided herein is compound of Formula (I), wherein the compound can be selected from:
4-(2-((2-(benzo[b]thiophen-3-yl)-6-(isopropylamino)pyrimidin-4-yl)amino)ethyl)phenol;
4-(2-((2-(benzo[b]thiophen-3-yl)-7-isopropylthieno[3,2-d]pyrimidin-4-yl)amino)ethyl)phenol;
4-(2-((2-(benzo[b]thiophen-3-yl)-7-isopropyl-6,7-dihydro-5H-pyrrolo[2,3-d]pyrimidin-4-yl)amino)ethyl)phenol;
2-(benzo[b]thiophen-3-yl)-4-((4-hydroxyphenethyl)amino)-7-isopropyl-5,7-dihydro-6H-pyrrolo[2,3-d]pyrimidin-6-one;
3-((2-(benzo[b]thiophen-3-yl)-9-isopropyl-9H-purin-6-yl)oxy)propanamide; 4-(2-((2-(benzo[b]thiophen-3-yl)-8-(dimethylamino)pyrimido[5,4-d]pyrimidin-4-yl)amino)ethyl)phenol;
5-(2-((2-(1H-indol-3-yl)ethyl)amino)-6-(sec-butylamino)pyrimidin-4-yl)nicotinonitrile;
N-(2-(1H-indol-3-yl)ethyl)-2-methyl-6-phenylthieno[2,3-d]pyrimidin-4-amine;
N-(2-(1H-indol-3-yl)ethyl)-6-(4-fluorophenyl)thieno[2,3-d]pyrimidin-4-amine;
3-(2-(benzo[b]thiophen-3-yl)-9-isopropyl-6-oxo-6,9-dihydro-1H-purin-1-yl)propanamide;
N-(2-(1H-indol-3-yl)ethyl)-2-(5-fluoropyridin-3-yl)quinazolin-4-amine;
5-(4-((2-(1H-indol-3-yl)ethyl)amino)quinazolin-2-yl)nicotinonitrile;
N⁴-(2-(1H-indol-3-yl)ethyl)-N²-(sec-butyl)quinazoline-2,4-diamine;
2-(benzo[b]thiophen-3-yl)-4-((4-hydroxyphenethyl)amino)-7-isopropyl-7H-pyrrolo[2,3-d]pyrimidine-5-carbonitrile;
N-(2-(1H-indol-3-yl)ethyl)-6-(benzo[b]thiophen-3-yl)-3-isopropylimidazo[1,5-a]pyrazin-8-amine;
4-(2-((6-(benzo[b]thiophen-3-yl)-3-isopropylimidazo[1,5-a]pyrazin-8-yl)amino)ethyl)phenol;
5-(4-((2-(1H-indol-3-yl)ethyl)amino)-7-isopropylthieno[3,2-d]pyrimidin-2-yl)nicotinonitrile;
N-(2-(1H-indol-3-yl)ethyl)-2-(5-fluoropyridin-3-yl)-7-isopropylthieno[3,2-d]pyrimidin-4-amine;
N-(2-(1H-indol-3-yl)ethyl)-2-(5-fluoropyridin-3-yl)furo[3,2-d]pyrimidin-4-amine;
N-(2-(1H-indol-3-yl)ethyl)-2-(5-methylpyridin-3-yl)furo[3,2-d]pyrimidin-4-amine;
N-(2-(1H-indol-3-yl)ethyl)-7-isopropyl-2-(5-methylpyridin-3-yl)thieno[3,2-d]pyrimidin-4-amine;
5-(4-((2-(1H-indol-3-yl)ethyl)amino)furo[3,2-d]pyrimidin-2-yl)nicotinonitrile; and
   pharmaceutically acceptable salts thereof.

### Formula (I-A)

In some embodiments provided herein, the compound of Formula (I) can have the structure of Formula (I-A):

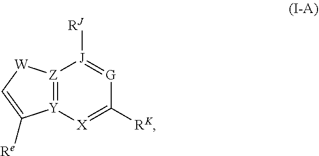

including pharmaceutically acceptable salts thereof, wherein: R^{J} can be —NR^{a}R^{b}, R^{a} can be hydrogen or C₁-C₄ alkyl; R^{b} can be R^{c} or —(C₁-C₄ alkyl)-R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: hydrogen, unsubstituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); Y and Z can each be C; X can be N or CH; W can be O or S; and R^{e} can be hydrogen or C₁-C₄ alkyl.

In some embodiments, R^{a} can be hydrogen. In other embodiments, R^{a} can be C₁-C₄ alkyl.

In some embodiments, R^{b} can be —(C₁-C₄ alkyl)-R^{c}. For example, R^{b} can be —CH₂—R^{c}, —CH₂CH₂—R^{c}, —CH₂CH₂CH₂—R^{c}, or —CH₂CH₂CH₂CH₂—R^{c}.

In some embodiments, R^{c} can be —OH. In some embodiments, R^{c} can be —O(C₁-C₄ alkyl). In some embodiments, R^{c} can be —O(C₁-C₄ haloalkyl). In some embodiments, R^{c} can be —C(═O)NH₂. In some embodiments, R^{c} can be unsubstituted C₆₋₁₀ aryl. In some embodiments, R^{c} can be substituted C₆₋₁₀ aryl. In some embodiments, R^{c} can be unsubstituted five- to ten-membered heteroaryl having 1-4 atoms selected from the group consisting of O, N, and S. In some embodiments, R^{c} can be substituted five- to ten-membered heteroaryl having 1-4 atoms selected from the group consisting of O, N, and S. In some embodiments, when a R^{c} moiety is indicated as substituted, the moiety can be substituted with one or more, for example, one, two, three, or four substituents E. In some embodiments, E can be —OH. In some embodiments, E can be C₁-C₄ alkyl. In some embodiments, E can be C₁-C₄ haloalkyl. In some embodiments, E can be —O(C₁-C₄ alkyl). In some embodiments, E can be —O(C₁-C₄ haloalkyl). In some embodiments R^{c} can be phenyl. In other embodiments, R^{c} can be hydroxyphenyl. In still other embodiments, R^{c} can be indolyl.

In some embodiments, R^{K} can be unsubstituted five- to ten-membered heteroaryl having 1-4 atoms selected from the group consisting of O, N, and S. In some embodiments, R^{K} can be substituted five- to ten-membered heteroaryl having 1-4 atoms selected from the group consisting of O, N, and S; wherein the substituted heteroaryl can substituted with one or more substituents Q, wherein each Q can independently selected from the group consisting of: —OH, C₁₋₄ alkyl, C₁₋₄ haloalkyl, halo, cyano, —O—(C₁₋₄ alkyl), and —O—(C₁₋₄ haloalkyl). In some embodiments, R^{K} can be pyridinyl. In other embodiments, R^{K} can be pyridinyl substituted with one or more substituents Q. For example, R^{K} can be methylpyridinyl, ethylpyridinyl cyanopyridinyl, chloropyridinyl, fluoropyridinyl, or bromopyridinyl.

In some embodiments, R^{e} can be hydrogen. In some embodiments, R^{e} can be C₁-C₄ alkyl. For example, R^{e} can be methyl, ethyl, n-propyl, iso-propyl, n-butyl, iso-butyl or tert-butyl.

In some embodiments, IV can be hydrogen; R^{b} can be —(C₁-C₄ alkyl)-R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein the substituted heteroaryl is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); and R^{e} can be C₁-C₄ alkyl.

In some embodiments, R^{a} can be hydrogen; R^{b} can be —(CH₂—CH₂)—R^{c}; R^{c} can be selected from the group consisting of: substituted phenyl and unsubstituted indolyl; wherein the substituted phenyl is substituted with one substituent E, wherein E can be —OH; R^{K} can be selected from the group consisting of: unsubstituted benzothiophenyl and substituted pyridinyl; wherein the substituted pyridinyl is substituted with one substituent Q, wherein Q can be selected from the group consisting of: C₁₋₄ alkyl, halo, and cyano; and R^{e} can be isopropyl.

In some embodiments, when W is O, R^{J} can be —NR^{a}R^{b}; R^{a} can be hydrogen; R^{b} can be —CH₂CH₂—R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, and —O(C₁-C₄ alkyl); R^{K} can be selected from the group consisting of unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —C₁₋₄ alkyl, halo, cyano, and —O—(C₁₋₄ alkyl); Y and Z can each be C; X can be N or CH; and R^{e} can be hydrogen or C₁-C₄ alkyl.

In some embodiments, when W is S, R^{J} can be —NR^{a}R^{b}; R^{a} can be hydrogen; R^{b} can be —CH₂CH₂—R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, and —O(C₁-C₄ alkyl); R^{K} can be selected from the group consisting of unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —C₁₋₄ alkyl, halo, cyano, and —O—(C₁₋₄ alkyl); Y and Z can each be C; X can be N or CH; and R^{e} can be hydrogen or C₁-C₄ alkyl.

In some embodiments, when R^{J} is —NR^{a}R^{b}; G can be N; W can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is C₁-C₄ alkyl; W can be S; R^{e} can be C₁-C₄ alkyl; J can be C; X can be N; Y can be C; and Z can be C. In some embodiments, the compound of Formula (I-A) can be N-(2-(1H-indol-3-yl)ethyl)-7-isopropyl-2-(5-methylpyridin-3-yl)thieno[3,2-d]pyrimidin-4-amine.

In some embodiments, when R^{J} is —NR^{a}R^{b}; G can be N; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is cyano; W can be S; R^{e} can be C₁-C₄ alkyl; J can be C; X can be N; Y can be C; and Z can be C. In some embodiments, the compound of Formula (I-A) can be 5-(4-((2-(1H-indol-3-yl)ethyl)amino)-7-isopropylthieno[3,2-d]pyrimidin-2-yl)nicotinonitrile.

In some embodiments, when R^{J} is —NR^{a}R^{b}; G can be N; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is halo; W can be S; R^{e} can be C₁-C₄ alkyl; J can be C; X can be N; Y can be C; and Z can be C. In some embodiments, the compound of Formula (I-A) can be N-(2-(1H-indol-3-yl)ethyl)-2-(5-fluoropyridin-3-yl)-7-isopropylthieno[3,2-d]pyrimidin-4-amine.

In some embodiments, when R^{J} is —NR^{a}R^{b}; G can be N; R^{a} can be hydrogen; R^{b} can be —CH₂CH₂—R^{c}, R^{c} can be substituted C₆₋₁₀ aryl, substituted with one or more E, wherein E can be —OH; R^{K} can be unsubstituted five- to ten-membered heteroaryl having 1-4 atoms selected from the group consisting of O, N, and S; W can be S; R^{e} can be C₁-C₄ alkyl; J can be C; X can be N; Y can be C; and Z can be C. In some embodiments, the compound of Formula (I-A) can be 4-(2-((2-(benzo[b]thiophen-3-yl)-7-isopropylthieno[3,2-d]pyrimidin-4-yl)amino)ethyl)phenol.

In some embodiments, when R^{J} is —NR^{a}R^{b}; G can be N; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is halo; W can be O; R^{e} can be hydrogen; J can be C; X can be N; Y can be C; and Z can be C. In some embodiments, the compound of Formula (I-A) can be N-(2-(1H-indol-3-yl)ethyl)-2-(5-fluoropyridin-3-yl)furo[3,2-d]pyrimidin-4-amine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is C₁-C₄ alkyl; W can be O; R^{e} can be hydrogen; J can be C; X can be N; Y can be C; and Z can be C. In some embodiments, the compound of Formula (I-A) can be N-(2-(1H-indol-3-yl)ethyl)-2-(5-methylpyridin-3-yl)furo[3,2-d]pyrimidin-4-amine.

In some embodiments, when R^{J} is —NR^{a}R^{b}; G is NR^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is cyano; W can be O; R^{e} can be hydrogen; J can be C; X can be N; Y can be C; and Z can be C. In some embodiments, the compound of Formula (I-A) can be 5-(4-((2-(1H-indol-3-yl)ethyl)amino)furo[3,2-d]pyrimidin-2-yl)nicotinonitrile.

In some embodiments, the compound of Formula (I-A), or a pharmaceutically acceptable salt thereof, can selected from the group consisting of:
N-(2-(1H-indol-3-yl)ethyl)-7-isopropyl-2-(5-methylpyridin-3-yl)thieno[3,2-d]pyrimidin-4-amine;
5-(4-((2-(1H-indol-3-yl)ethyl)amino)-7-isopropylthieno[3,2-d]pyrimidin-2-yl)nicotinonitrile; N-(2-(1H-indol-3-yl)ethyl)-2-(5-fluoropyridin-3-yl)-7-isopropylthieno[3,2-d]pyrimidin-4-amine;
4-(2-((2-(benzo[b]thiophen-3-yl)-7-isopropylthieno[3,2-d]pyrimidin-4-yl)amino)ethyl)phenol;
N-(2-(1H-indol-3-yl)ethyl)-2-(5-fluoropyridin-3-yl)furo[3,2-d]pyrimidin-4-amine;
N-(2-(1H-indol-3-yl)ethyl)-2-(5-methylpyridin-3-yl)furo[3,2-d]pyrimidin-4-amine; and
5-(4-((2-(1H-indol-3-yl)ethyl)amino)furo[3,2-d]pyrimidin-2-yl)nicotinonitrile. Formula (I-B)

In other embodiments provided herein, the compound of Formula (I) can have the structure of Formula (I-B):

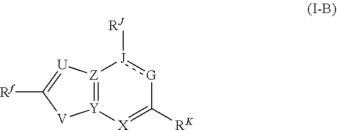

including pharmaceutically acceptable salts thereof, wherein: R^{a} can be hydrogen or C₁-C₄ alkyl; R^{b} can be R^{c} or —(C₁₋₄ alkyl)-R^{c}; R^{c} can be selected from the group consisting of: —OH, —O(C₁-C₄ alkyl), —O(C₁-C₄ haloalkyl); —C(═O)NH₂; unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: hydrogen, unsubstituted C₁₋₆ alkyl; substituted C₁₋₆ alkyl; —NH(C₁₋₄ alkyl); —N(C₁₋₄ alkyl)₂, unsubstituted C₆₋₁₀ aryl; substituted C₆₋₁₀-aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); R^{G} can be selected from the group consisting of hydrogen, C₁₋₄ alkyl, and —(C₁₋₄ alkyl)-C(═O)NH₂; R^{f} can be selected from the group consisting of hydrogen, C₁₋₄ alkyl, unsubstituted C₆-C₁₀ aryl, and C₆-C₁₀ aryl substituted with 1-5 halo atoms; U can be N or CR^{U}; V can be S or NR^{V}; R^{U} can be selected from the group consisting of hydrogen, C₁₋₄ alkyl, halo, and cyano; R^{V} can be hydrogen or C₁-C₄ alkyl; wherein when U is CR^{U} and V is NR^{V}, R^{U} is selected from the group consisting of C₁₋₄ alkyl, halo, and cyano; Y and Z can each be C; and X can be N or CH.

In some embodiments, R^{a} can be hydrogen. In other embodiments, R^{a} can be C₁-C₄ alkyl.

In some embodiments, R^{b} can be —(C₁-C₄ alkyl)-R^{c}. For example, R^{b} can be —CH₂—R^{c}, —CH₂CH₂—R^{c}, —CH₂CH₂CH₂—R^{c}, or —CH₂CH₂CH₂CH₂—R^{c}. In certain embodiments, R^{b} can be —(CH₂CH₂)—R^{c}. In certain embodiments, R^{b} can be —(CH₂CH₂)—C(═O)NH₂. In certain embodiments, R^{b} can be —(CH₂CH₂)-(indolyl). In certain embodiments, R^{b} can be —(CH₂CH₂)-(hydroxyphenyl).

In some embodiments, R^{c} can be —OH. In some embodiments, R^{c} can be —O(C₁-C₄ alkyl). In some embodiments, R^{c} can be —O(C₁-C₄ haloalkyl). In some embodiments, R^{c} can be —C(═O)NH₂. In some embodiments, R^{c} can be unsubstituted C₆₋₁₀ aryl. In some embodiments, R^{c} can be substituted C₆₋₁₀ aryl. In some embodiments, R^{c} can be unsubstituted five- to ten-membered heteroaryl having 1-4 atoms selected from the group consisting of O, N, and S. In some embodiments, R^{c} can be substituted five- to ten-membered heteroaryl having 1-4 atoms selected from the group consisting of O, N, and S. In some embodiments, when a R^{c} moiety is indicated as substituted, the moiety can be substituted with one or more, for example, one, two, three, or four substituents E. In some embodiments, E can be —OH. In some embodiments, E can be C₁-C₄ alkyl. In some embodiments, E can be C₁-C₄ haloalkyl. In some embodiments, E can be —O(C₁-C₄ alkyl). In some embodiments, E can be —O(C₁-C₄ haloalkyl).

In some embodiments, R^{K} can be hydrogen. In other embodiments, R^{K} can be C₁-C₄ alkyl. For example, R^{K} can be methyl, ethyl, n-propyl, iso-propyl, n-butyl, iso-butyl or tert-butyl. In some embodiments, R^{K} can be selected from the group consisting of: unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein the substituted heteroaryl can substituted with one or more substituents Q, wherein each Q can independently selected from the group consisting of: —OH, C₁₋₄ alkyl, C₁₋₄ haloalkyl, halo, cyano, —O—(C₁₋₄ alkyl), and —O—(C₁₋₄ haloalkyl). In certain embodiments, R^{K} can be benzothiophenyl. In other embodiments, R^{K} can be pyridinyl substituted with one or more substituents Q. For example, R^{K} can be methylpyridinyl, ethylpyridinyl cyanopyridinyl, chloropyridinyl, fluoropyridinyl, or bromopyridinyl.

In some embodiments, R^{G} can be selected from the group consisting of hydrogen, C₁₋₄ alkyl, and —(C₁₋₄ alkyl)-C(═O)NH₂. In certain embodiments, R^{G} can be —(CH₂CH₂)—C(═O)NH₂.

In some embodiments, R^{f} can be hydrogen. In other embodiments, R^{f} can be C₁₋₄ alkyl. For example, R^{f} can be methyl, ethyl, n-propyl, iso-propyl, n-butyl, iso-butyl or tert-butyl. In some embodiments, R^{f} can be unsubstituted C₆-C₁₀ aryl. In other embodiments, R^{f} can be C₆-C₁₀ aryl substituted with 1-5 halo atoms. In certain embodiments, R^{f} can be phenyl substituted with 1-5 halo atoms. In certain embodiments, R^{f} can be fluorophenyl.

In some embodiments, U can be N. In other embodiments, U can be CR^{U}.

In some embodiments, V can be S. In other embodiments, V can be NR^{V}.

In some embodiments, R^{U} can be hydrogen. In some embodiments, R^{U} can be C₁₋₄ alkyl. In other embodiments R^{U} can be halo. For example, R^{U} can be fluoro, chloro, bromo, or iodo. In still other embodiments, R^{U} can be cyano.

In some embodiments, R^{V} can be hydrogen. In other embodiments, R^{V} can be C₁₋₄ alkyl. For example, R^{V} can be methyl, ethyl, n-propyl, iso-propyl, n-butyl, iso-butyl or tert-butyl. In some embodiments, Y and Z can each be C and X can be N. In other embodiments, Y and Z can each be C and X can be CH.

In some embodiments, R^{a} can be hydrogen; R^{b} can be —(C₁₋₄ alkyl)-R^{c}; R^{c} can be selected from the group consisting of: —C(═O)NH₂, unsubstituted C₆₋₁₀ aryl; substituted C₆₋₁₀-aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted can be substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein the substituted heteroaryl is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); R^{G} is C₁₋₄ alkyl or —(C₁₋₄ alkyl)-C(═O)NH₂; R^{f} can be selected from the group consisting of hydrogen, unsubstituted phenyl, and phenyl substituted with 1-5 halo atoms; Y and Z each can be C; and X can be CH.

In some embodiments, R^{a} can be hydrogen; R^{b} can be —(CH₂—CH₂)—R^{c}; R^{c} can be selected from the group consisting of: —C(═O)NH₂, substituted phenyl and unsubstituted indolyl; wherein the substituted phenyl is substituted with one substituent E, wherein E can be —OH; R^{K} can be selected from the group consisting of: unsubstituted benzothiohenyl and substituted pyridinyl; wherein the substituted pyridinyl is substituted with one substituent Q, wherein Q can be selected from the group consisting of: C₁₋₄ alkyl, halo, and cyano; R^{G} can be —(CH₂CH₂)—C(═O)NH₂; R^{f} can be selected from the group consisting of hydrogen, phenyl, and fluorophenyl; Y and Z each can be C; and X can be CH.

In some embodiments, when V is S, R^{a} can be hydrogen or C₁-C₄ alkyl; R^{b} can be R^{c} or —(CH₂—CH₂)—R^{c}; R^{c} can be selected from the group consisting of: —C(═O)NH₂; unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, and —O(C₁-C₄ alkyl); R^{K} can be selected from the group consisting of: hydrogen, unsubstituted C₁₋₆ alkyl; substituted C₁₋₆ alkyl; —NH(C₁₋₄ alkyl); and —N(C₁₋₄ alkyl)₂; wherein a R^{K} moiety indicated as substituted is substituted with one or more substituents Q, wherein each Q can be independently selected from the group consisting of: —OH, C₁₋₄ alkyl, halo, cyano, and —O—(C₁₋₄ alkyl; R^{G} can be selected from the group consisting of hydrogen, C₁₋₄ alkyl, and —(C₁₋₄ alkyl)-C(═O)NH₂; R^{f} can be selected from the group consisting of hydrogen, C₁₋₄ alkyl, unsubstituted C₆-C₁₀ aryl, and C₆-C₁₀ aryl substituted with 1-5 halo atoms; U can be CR^{U}; R^{U} can be selected from the group consisting of hydrogen, C₁₋₄ alkyl, halo, and cyano; Y and Z can each be C; and X can be N.

In some embodiments, when V is NR^{V}, R^{a} can be hydrogen or C₁-C₄ alkyl; R^{b} can be R^{c} or —(CH₂—CH₂)—R^{c}; R^{c} can be selected from the group consisting of: —C(═O)NH₂; unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄, and —O(C₁-C₄ alkyl); R^{K} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, halo, cyano, and —O—(C₁₋₄ alkyl); R^{G} can be selected from the group consisting of hydrogen, C₁₋₄ alkyl, and —(C₁₋₄ alkyl)-C(═O)NH₂; R^{f} can be hydrogen; U can be N or CR^{U}; R^{U} can be selected from the group consisting of C₁₋₄ alkyl, halo, and cyano; R^{V} can be hydrogen or C₁-C₄ alkyl; Y and Z can each be C; and X can be N or CH.

In some embodiments, when R^{J} is —OR^{b}; G can be N; [symbol] joining G and J can be a double bond; R^{b} can be —CH₂CH₂—R^{c}; R^{c} can be —C(═O)NH₂; R^{K} can unsubstituted five- to ten-membered heteroaryl having 1-4 atoms selected from the group consisting of O, N, and S; U can N; V can be NR^{v}; R^{v} can be C₁-C₄ alkyl; R^{f} can be hydrogen; J can be C; X can be N; Y can be C; and Z can be C. In some embodiments, the compound of Formula (I-B) can be 3-((2-(benzo[b]thiophen-3-yl)-9-isopropyl-9H-purin-6-yl)oxy)propanamide.

In some embodiments, when R^{J} is ═O; G can be N substituted with R^{G}; [symbol] joining G and J can be a single bond; R^{G} can be —(C₁₋₄ alkyl)-C(═O)NH₂; R^{K} can be unsubstituted five- to ten-membered heteroaryl having 1-4 atoms selected from the group consisting of O, N, and S; U can N; V can be NR^{v}; R^{v} can be C₁-C₄ alkyl; R^{f} can be hydrogen; J can be C; X can be N; Y can be C; and Z can be C. In some embodiments, the compound of Formula (I-B) can be 3-(2-(benzo[b]thiophen-3-yl)-9-isopropyl-6-oxo-6,9-dihydro-1H-purin-1-yl)propanamide.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be substituted C₆₋₁₀ aryl, substituted with one or more E, wherein E is —OH; R^{K} can be unsubstituted five- to ten-membered heteroaryl having 1-4 atoms selected from the group consisting of O, N, and S; U can be CR^{u}; R^{u} can be cyano; V can be NR^{v}; R^{v} can be C₁-C₄ alkyl; R^{f} can be hydrogen; J can be C; X can be N; Y can be C; and Z can be C. In some embodiments, the compound of Formula (I-B) can be 2-(benzo[b]thiophen-3-yl)-4-((4-hydroxyphenethyl)amino)-7-isopropyl-7H-pyrrolo[2,3-d]pyrimidine-5-carbonitrile.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be unsubstituted C₁₋₆ alkyl; U can be CR^{u}; R^{u} can be hydrogen; V can be S; R^{f} can be phenyl; J can be C; X can be N; Y can be C; Z can be C. In some embodiments, the compound of Formula (I-B) can be N-(2-(1H-indol-3-yl)ethyl)-2-methyl-6-phenylthieno[2,3-d]pyrimidin-4-amine.

In some embodiments, when R^{J} can be —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be hydrogen; U can be CR^{u}; R^{u} can be hydrogen; V can be S; R^{f} can be fluorophenyl; J can be C; X can be N; Y can be C; and Z can be C. In some embodiments, the compound of Formula (I-B) can be N-(2-(1H-indol-3-yl)ethyl)-6-(4-fluorophenyl)thieno[2,3-d]pyrimidin-4-amine.

In some embodiments, the compound of Formula (I-B), or a pharmaceutically acceptable salt thereof, can selected from the group consisting of:
3-((2-(benzo[b]thiophen-3-yl)-9-isopropyl-9H-purin-6-yl)oxy)propanamide;
3-(2-(benzo[b]thiophen-3-yl)-9-isopropyl-6-oxo-6,9-dihydro-1H-purin-1-yl)propanamide;
2-(benzo[b]thiophen-3-yl)-4-((4-hydroxyphenethyl)amino)-7-isopropyl-7H-pyrrolo[2,3-d]pyrimidine-5-carbonitrile;
N-(2-(1H-indol-3-yl)ethyl)-2-methyl-6-phenylthieno[2,3-d]pyrimidin-4-amine; and
N-(2-(1H-indol-3-yl)ethyl)-6-(4-fluorophenyl)thieno[2,3-d]pyrimidin-4-amine.

### Formula (I-C)

In still other embodiments provided herein, the compound of Formula (I) can have the structure of Formula (I-C):

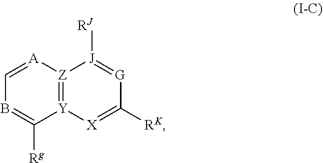

including pharmaceutically acceptable salts thereof, wherein: R^{J} can be —NR^{a}R^{b}, R^{a} can be hydrogen or C₁-C₄ alkyl; R^{b} can be R^{c} or —(C₁-C₄ alkyl)-R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: hydrogen, unsubstituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); A can be N or CH; B can be N or CH; R^{g} can be selected from the group consisting of hydrogen, C₁₋₄ alkyl, and —N(C₁₋₄ alkyl)₂; Y and Z can each be C; and X can be N or CH.

In some embodiments, R^{K} can be —NH(C₁₋₄ alkyl). For example, in some embodiments, R^{K} can be —NH(CH₃), —NH(CH₂CH₃), —NH(isopropyl), or —NH(sec-butyl). In some embodiments, R^{K} can be unsubstituted benzothiophenyl. In other embodiments, R^{K} can be substituted pyridinyl. For example, R^{K} can be methylpyridinyl, ethylpyridinyl, cyanopyridinyl, chloropyridinyl, fluoropyridinyl, or bromopyridinyl.

In some embodiments, A can be N and B can be N. In other embodiments, A can be N and B can be CH. In still other embodiments, A can be CH and B can be N. In yet still other embodiments, A can be CH and B can be CH.

In some embodiments, W can be hydrogen. In other embodiments, W can be —N(C₁₋₄ alkyl)₂. In certain embodiments, R^{g} can be —N(CH₃)₂.

In some embodiments, R^{a} can be hydrogen; R^{b} can be —(C₁-C₄ alkyl)-R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: —NH(C₁₋₄ alkyl); unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein the substituted heteroaryl is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); and W can be hydrogen or —N(C₁₋₄ alkyl)₂.

In some embodiments, R^{a} can be hydrogen; R^{b} can be —(C₁-C₄ alkyl)-R^{c}; R^{c} can be selected from the group consisting of: substituted phenyl and unsubstituted indolyl; wherein the substituted phenyl is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: —NH(C₁₋₄ alkyl); unsubstituted benzothiophenyl; and substituted pyridinyl; wherein the substituted pyridinyl is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); and W can be hydrogen or —N(C₁₋₄ alkyl)₂.

In some embodiments, R^{a} can be hydrogen; R^{b} can be —(CH₂CH₂)—R^{c}; R^{c} can be selected from the group consisting of: substituted phenyl and unsubstituted indolyl; wherein the substituted phenyl is substituted with one substituent E, wherein E can be —OH; R^{K} can be selected from the group consisting of: —NH(sec-butyl); unsubstituted benzothiohenyl, and substituted pyridinyl; wherein the substituted pyridinyl is substituted with one or more substituents Q, wherein each Q can be independently selected from the group consisting of: C₁₋₄ alkyl, halo, and cyano; and W can be hydrogen or —N(CH₃)₂.

In some embodiments, when A is C and B is C, R^{J} can be —NR^{a}R^{b}; G can be N; R^{a} can be hydrogen; R^{b} can be —CH₂CH₂—R^{c}; R^{c} can be substituted C₆₋₁₀-aryl, substituted with one or more E, wherein E is —OH; or unsubstituted five- to ten-membered heteroaryl having 1-4 atoms selected from the group consisting of O, N, and S; R^{K} can be unsubstituted five- to ten-membered heteroaryl having 1-4 atoms selected from the group consisting of O, N, and S; R^{g} can be hydrogen; J can be C; X can be N; Y can be C; and Z is C.

In some embodiments, when R^{J} is —NR^{a}R^{b}; G can be N; R^{a} can be hydrogen; R^{b} can be —CH₂CH₂—R^{c}; R^{c} can be substituted C₆₋₁₀ aryl, substituted with one or more E, wherein E is —OH; R^{K} is unsubstituted five- to ten-membered heteroaryl having 1-4 atoms selected from the group consisting of O, N, and S; A can be N; B can be N; W can be —N(C₁₋₄ alkyl)₂; J can be C; X can be N; Y can be C; and Z is C. In some embodiments, the compound of Formula (I-C) can be 4-(2-((2-(benzo[b]thiophen-3-yl)-8-(dimethylamino)pyrimido[5,4-d]pyrimidin-4-yl)amino)ethyl)phenol.

In some embodiments, when R^{J} is —NR^{a}R^{b}; G can be N; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q can be halo; A can be CH; B can be CH; W can be hydrogen; J can be C; X can be N; Y can be C; and Z can be C. In some embodiments, the compound of Formula (I-C) can be N-(2-(1H-indol-3-yl)ethyl)-2-(5-fluoropyridin-3-yl)quinazolin-4-amine.

In some embodiments, when R^{J} is —NR^{a}R^{b}; G is N; [symbol] joining G and J can be a double bond; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q can be cyano; A can be CH; B can be CH; R^{g} can be hydrogen; J can be C; X can be N; Y can be C; and Z can be C. In some embodiments, the compound of Formula (I-C) can be 5-(4-((2-(1H-indol-3-yl)ethyl)amino)quinazolin-2-yl)nicotinonitrile.

In some embodiments, when R^{J} is —NR^{a}R^{b}; G can be N; [symbol] joining G and J can be a double bond; R^{a} can be hydrogen R″ can be —CH₂CH₂—R^{c}; R^{c} can be unsubstituted five- to ten-membered heteroaryl having 1-4 atoms selected from the group consisting of O, N, and S; R^{K} can be —NH(C₁₋₄ alkyl); A can be CH; B can be CH; W can be hydrogen; J can be C; X can be N; Y can be C; and Z can be C. In some embodiments, the compound of Formula (I-C) can be N⁴-(2-(1H-indol-3-yl)ethyl)-N²-(sec-butyl)quinazoline-2,4-diamine.

In some embodiments, the compound of Formula (I-C), or a pharmaceutically acceptable salt thereof, can selected from the group consisting of:
4-(2-((2-(benzo[b]thiophen-3-yl)-8-(dimethylamino)pyrimido[5,4-d]pyrimidin-4-yl)amino)ethyl)phenol;
N-(2-(1H-indol-3-yl)ethyl)-2-(5-fluoropyridin-3-yl)quinazolin-4-amine; 5-(4-((2-(1H-indol-3-yl)ethyl)amino)quinazolin-2-yl)nicotinonitrile; and
N⁴-(2-(1H-indol-3-yl)ethyl)-N²-(sec-butyl)quinazoline-2,4-diamine.

### Formula (I-D)

In yet still other embodiments provided herein, the compound of Formula (I) can have the structure of Formula (I-D):

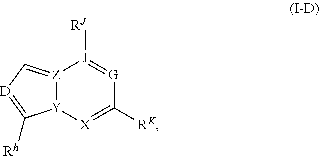

including pharmaceutically acceptable salts thereof, wherein: R^{J} can be —NR^{a}R^{b}; R^{a} can be hydrogen or C₁-C₄ alkyl; R^{b} can be R^{c} or —(C₁₋₄ alkyl)-R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); R^{h} can be hydrogen or C₁₋₄ alkyl; D can be N or CH; Y can be N; Z can be C; and X can be N or CH.

In some embodiments, R^{h} can be hydrogen. In other embodiments, R^{h} can be C₁₋₄ alkyl. For example, R^{h} can be methyl, ethyl, n-propyl, iso-propyl, n-butyl, iso-butyl or tert-butyl.

In some embodiments, D can be N. In other embodiments, D can be CH.

In some embodiments, when D is N, Y can be N, Z can be C, and X can be N. In other embodiments, when D is N, Y can be N, Z can be C, and X can be CH. In some embodiments, when D is CH, Y can be N, Z can be C, and X can be N. In other embodiments, when D is CH, Y can be N, Z can be C, and X can be CH.

In some embodiments, R^{a} can be hydrogen; R^{b} can be —(C₁₋₄ alkyl)-R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); and R^{h} can be hydrogen or C₁₋₄ alkyl.

In some embodiments, R^{a} can be hydrogen; R^{b} can be —(C₁-C₄ alkyl)-R^{c}; R^{c} can be selected from the group consisting of: substituted phenyl and unsubstituted indolyl; wherein the substituted phenyl is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be unsubstituted benzothiophenyl; and R^{h} can be hydrogen or C₁₋₄ alkyl.

In some embodiments, R^{a} can be hydrogen; R^{b} can be —(CH₂—CH₂)—R^{c}; R^{c} can be selected from the group consisting of: substituted phenyl and unsubstituted indolyl; wherein the substituted phenyl is substituted with one substituent E, wherein E can be —OH; R^{K} can be unsubstituted benzothiophenyl; and R^{h} can be hydrogen or C₁₋₄ alkyl.

In some embodiments, when D is N; R^{J} is —NR^{a}R^{b}; G can be N; W can be hydrogen; R^{b} can be —CH₂CH₂—R^{c}; R^{c} can be unsubstituted five- to ten-membered heteroaryl having 1-4 atoms selected from the group consisting of O, N, and S; or substituted C₆₋₁₀ aryl, substituted with one or more E, wherein E is —OH; R^{K} can be unsubstituted five- to ten-membered heteroaryl having 1-4 atoms selected from the group consisting of O, N, and S; R^{h} can be C₁₋₄ alkyl; J can be C; X can be C; Y can be N; and Z can be C; wherein the valency of any carbon atom is filled as needed with hydrogen atoms.

In some embodiments, when R^{J} is —NR^{a}R^{b}; G can be N; R^{a} can be hydrogen; R^{b} can be —CH₂CH₂—R^{c}; R^{c} can be unsubstituted five- to ten-membered heteroaryl having 1-4 atoms selected from the group consisting of O, N, and S or substituted C₆₋₁₀ aryl, substituted with one or more E, wherein E is —OH; R^{K} can be unsubstituted five- to ten-membered heteroaryl having 1-4 atoms selected from the group consisting of O, N, and S; D can be N; R^{h} can be C₁₋₄ alkyl; J can be C; X can be C; Y can be N; and Z can be C; wherein the valency of any carbon atom is filled as needed with hydrogen atoms. In some embodiments, the compound of Formula (I-D) can be N-(2-(1H-indol-3-yl)ethyl)-6-(benzo[b]thiophen-3-yl)-3-isopropylimidazo[1,5-a]pyrazin-8-amine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be substituted C₆₋₁₀-aryl, substituted with one or more E, wherein E is —OH; R^{K} can be unsubstituted five- to ten-membered heteroaryl having 1-4 atoms selected from the group consisting of O, N, and S; D can be N; R^{h} can be C₁₋₄ alkyl; J can be C; X can be C; Y can be N; and Z can be C; wherein the valency of any carbon atom is filled as needed with hydrogen atoms. In some embodiments, the compound of Formula (I-D) can be 4-(2-((6-(benzo[b]thiophen-3-yl)-3-isopropylimidazo[1,5-a]pyrazin-8-yl)amino)ethyl)phenol.

In some embodiments, the compound of Formula (I-D), or a pharmaceutically acceptable salt thereof, can selected from the group consisting of: N-(2-(1H-indol-3-yl)ethyl)-6-(benzo[b]thiophen-3-yl)-3-isopropylimidazo[1,5-a]pyrazin-8-amine; and 4-(2-((6-(benzo[b]thiophen-3-yl)-3-isopropylimidazo[1,5-a]pyrazin-8-yl)amino)ethyl)phenol.

The compounds provided herein may be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of skill in the art will recognize that administration of a compound in its (R) form is equivalent, for compounds that undergo epimerization in vivo, to administration of the compound in its (5) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

5.4. Isolation of NK Cells

Methods of isolating natural killer cells are known in the art and can be used to isolate the natural killer cells, e.g., NK cells produced using the three-stage method, described herein. For example, NK cells can be isolated or enriched, for example, by staining cells, in one embodiment, with antibodies to CD56 and CD3, and selecting for CD56⁺CD3⁻ cells. In certain embodiments, the NK cells are enriched for CD56⁺CD3⁻ cells in comparison with total cells produced using the three-stage method, described herein. NK cells, e.g., cells produced using the three-stage method, described herein, can be isolated using a commercially available kit, for example, the NK Cell Isolation Kit (Miltenyi Biotec). NK cells, e.g., cells produced using the three-stage method, described herein, can also be isolated or enriched by removal of cells other than NK cells in a population of cells that comprise the NK cells, e.g., cells produced using the three-stage method, described herein. For example, NK cells, e.g., cells produced using the three-stage method, described herein, may be isolated or enriched by depletion of cells displaying non-NK cell markers using, e.g., antibodies to one or more of CD3, CD4, CD14, CD19, CD20, CD36, CD66b, CD123, HLA DR and/or CD235a (glycophorin A). Negative isolation can be carried out using a commercially available kit, e.g., the NK Cell Negative Isolation Kit (Dynal Biotech). Cells isolated by these methods may be additionally sorted, e.g., to separate CD11a+ and CD11a− cells, and/or CD117+ and CD117− cells, and/or CD16⁺ and CD16⁻ cells, and/or CD94⁺ and CD94⁻. In certain embodiments, cells, e.g., cells produced by the three-step methods described herein, are sorted to separate CD11a+ and CD11a− cells. In specific embodiments, CD11a+ cells are isolated. In certain embodiments, the cells are enriched for CD11a⁺ cells in comparison with total cells produced using the three-stage method, described herein. In specific embodiments, CD11a− cells are isolated. In certain embodiments, the cells are enriched for CD11a− cells in comparison with total cells produced using the three-stage method, described herein. In certain embodiments, cells are sorted to separate CD117+ and CD117− cells. In specific embodiments, CD117+ cells are isolated. In certain embodiments, the cells are enriched for CD117⁺ cells in comparison with total cells produced using the three-stage method, described herein. In specific embodiments, CD117− cells are isolated. In certain embodiments, the cells are enriched for CD117− cells in comparison with total cells produced using the three-stage method, described herein. In certain embodiments, cells are sorted to separate CD16⁺ and CD16⁻ cells. In specific embodiments, CD16⁺ cells are isolated. In certain embodiments, the cells are enriched for CD16⁺ cells in comparison with total cells produced using the three-stage method, described herein. In specific embodiments, CD16⁻ cells are isolated. In certain embodiments, the cells are enriched for CD16− cells in comparison with total cells produced using the three-stage method, described herein. In certain embodiments, cells are sorted to separate CD94⁺ and CD94⁻ cells. In specific embodiments, CD94⁺ cells are isolated. In certain embodiments, the cells are enriched for CD94⁺ cells in comparison with total cells produced using the three-stage method, described herein. In specific embodiments, CD94⁻ cells are isolated. In certain embodiments, the cells are enriched for CD94− cells in comparison with total cells produced using the three-stage method, described herein. In certain embodiments, isolation is performed using magnetic separation. In certain embodiments, isolation is performed using flow cytometry.

Methods of isolating ILC3 cells are known in the art and can be used to isolate the ILC3 cells, e.g., ILC3 cells produced using the three-stage method, described herein. For example, ILC3 cells can be isolated or enriched, for example, by staining cells, in one embodiment, with antibodies to CD56, CD3, and CD11a, and selecting for CD56⁺CD3⁻CD11a⁻ cells. ILC3 cells, e.g., cells produced using the three-stage method, described herein, can also be isolated or enriched by removal of cells other than ILC3 cells in a population of cells that comprise the ILC3 cells, e.g., cells produced using the three-stage method, described herein. For example, ILC3 cells, e.g., cells produced using the three-stage method, described herein, may be isolated or enriched by depletion of cells displaying non-ILC3 cell markers using, e.g., antibodies to one or more of CD3, CD4, CD11a, CD14, CD19, CD20, CD36, CD66b, CD94, CD123, HLA DR and/or CD235a (glycophorin A). Cells isolated by these methods may be additionally sorted, e.g., to separate CD117⁺ and CD117⁻ cells. NK cells can be isolated or enriched, for example, by staining cells, in one embodiment, with antibodies to CD56, CD3, CD94, and CD11a, and selecting for CD56⁺CD3⁻CD94⁺CD11⁺ cells. NK cells, e.g., cells produced using the three-stage method, described herein, can also be isolated or enriched by removal of cells other than NK cells in a population of cells that comprise the NK cells, e.g., cells produced using the three-stage method, described herein. In certain embodiments, the NK cells are enriched for CD56⁺CD3⁻CD94⁺CD11a⁺ cells in comparison with total cells produced using the three-stage method, described herein.

In one embodiment, ILC3 cells are isolated or enriched by selecting for CD56⁺CD3⁻CD11a⁻ cells. In certain embodiments, the ILC3 cells are enriched for CD56⁺CD3⁻CD11a⁻ cells in comparison with total cells produced using the three-stage method, described herein. In one embodiment, ILC3 cells are isolated or enriched by selecting for CD56⁺CD3⁻CD11a⁻CD117+ cells. In certain embodiments, the ILC3 cells are enriched for CD56⁺CD3⁻CD11a⁻CD117+ cells in comparison with total cells produced using the three-stage method, described herein. In one embodiment, ILC3 cells are isolated or enriched by selecting for CD56⁺CD3⁻CD11a⁻CD117⁺CDIL1R1⁺ cells. In certain embodiments, the ILC3 cells are enriched for CD56⁺CD3⁻CD11a⁻CD117⁺CDIL1R1⁺ cells in comparison with total cells produced using the three-stage method, described herein.

In one embodiment, NK cells are isolated or enriched by selecting for CD56⁺CD3⁻CD94⁺CD11a⁺ cells. In certain embodiments, the NK cells are enriched for CD56+CD3⁻CD94+CD11a⁺ cells in comparison with total cells produced using the three-stage method, described herein. In one embodiment, NK cells are isolated or enriched by selecting for CD56⁺CD3⁻CD94⁺CD11a⁺CD117⁻ cells. In certain embodiments, the NK cells are enriched for CD56⁺CD3⁻CD94⁺CD11a⁺CD117⁻ cells in comparison with total cells produced using the three-stage method, described herein.

Cell separation can be accomplished by, e.g., flow cytometry, fluorescence-activated cell sorting (FACS), or, in one embodiment, magnetic cell sorting using microbeads conjugated with specific antibodies. The cells may be isolated, e.g., using a magnetic activated cell sorting (MACS) technique, a method for separating particles based on their ability to bind magnetic beads (e.g., about 0.5-100 μm diameter) that comprise one or more specific antibodies, e.g., anti-CD56 antibodies. Magnetic cell separation can be performed and automated using, e.g., an AUTOMACS™ Separator (Miltenyi). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

5.5. Placental Perfusate

NK cells and/or ILC3 cells, e.g., NK cell and/or ILC3 cell populations produced according to the three-stage method described herein may be produced from hematopoietic cells, e.g., hematopoietic stem or progenitors from any source, e.g., placental tissue, placental perfusate, umbilical cord blood, placental blood, peripheral blood, spleen, liver, or the like. In certain embodiments, the hematopoietic stem cells are combined hematopoietic stem cells from placental perfusate and from cord blood from the same placenta used to generate the placental perfusate. Placental perfusate comprising placental perfusate cells that can be obtained, for example, by the methods disclosed in U.S. Pat. Nos. 7,045,148 and 7,468,276 and U.S. Patent Application Publication No. 2009/0104164, the disclosures of which are hereby incorporated in their entireties.

5.5.1. Cell Collection Composition

The placental perfusate and perfusate cells, from which hematopoietic stem or progenitors may be isolated, or useful in tumor suppression or the treatment of an individual having tumor cells, cancer or a viral infection, e.g., in combination with the NK cells and/or ILC3 cells, e.g., NK cell and/or ILC3 cell populations produced according to the three-stage method provided herein, can be collected by perfusion of a mammalian, e.g., human post-partum placenta using a placental cell collection composition. Perfusate can be collected from the placenta by perfusion of the placenta with any physiologically-acceptable solution, e.g., a saline solution, culture medium, or a more complex cell collection composition. A cell collection composition suitable for perfusing a placenta, and for the collection and preservation of perfusate cells is described in detail in related U.S. Application Publication No. 2007/0190042, which is incorporated herein by reference in its entirety.

The cell collection composition can comprise any physiologically-acceptable solution suitable for the collection and/or culture of stem cells, for example, a saline solution (e.g., phosphate-buffered saline, Kreb's solution, modified Kreb's solution, Eagle's solution, 0.9% NaCl. etc.), a culture medium (e.g., DMEM, H.DMEM, etc.), and the like.

The cell collection composition can comprise one or more components that tend to preserve placental cells, that is, prevent the placental cells from dying, or delay the death of the placental cells, reduce the number of placental cells in a population of cells that die, or the like, from the time of collection to the time of culturing. Such components can be, e.g., an apoptosis inhibitor (e.g., a caspase inhibitor or JNK inhibitor); a vasodilator (e.g., magnesium sulfate, an antihypertensive drug, atrial natriuretic peptide (ANP), adrenocorticotropin, corticotropin-releasing hormone, sodium nitroprusside, hydralazine, adenosine triphosphate, adenosine, indomethacin or magnesium sulfate, a phosphodiesterase inhibitor, etc.); a necrosis inhibitor (e.g., 2-(1H-Indol-3-yl)-3-pentylamino-maleimide, pyrrolidine dithiocarbamate, or clonazepam); a TNF-α inhibitor; and/or an oxygen-carrying perfluorocarbon (e.g., perfluorooctyl bromide, perfluorodecyl bromide, etc.).

The cell collection composition can comprise one or more tissue-degrading enzymes, e.g., a metalloprotease, a serine protease, a neutral protease, a hyaluronidase, an RNase, or a DNase, or the like. Such enzymes include, but are not limited to, collagenases (e.g., collagenase I, II, III or IV, a collagenase from Clostridium histolyticum, etc.); dispase, thermolysin, elastase, trypsin, LIBERASE, hyaluronidase, and the like.

The cell collection composition can comprise a bacteriocidally or bacteriostatically effective amount of an antibiotic. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram(+) and/or Gram(—) bacteria, e.g., Pseudomonas aeruginosa, Staphylococcus aureus, and the like.

The cell collection composition can also comprise one or more of the following compounds: adenosine (about 1 mM to about 50 mM); D-glucose (about 20 mM to about 100 mM); magnesium ions (about 1 mM to about 50 mM); a macromolecule of molecular weight greater than 20,000 daltons, in one embodiment, present in an amount sufficient to maintain endothelial integrity and cellular viability (e.g., a synthetic or naturally occurring colloid, a polysaccharide such as dextran or a polyethylene glycol present at about 25 g/l to about 100 g/l, or about 40 g/l to about 60 g/l); an antioxidant (e.g., butylated hydroxyanisole, butylated hydroxytoluene, glutathione, vitamin C or vitamin E present at about 25 μM to about 100 μM); a reducing agent (e.g., N-acetylcysteine present at about 0.1 mM to about 5 mM); an agent that prevents calcium entry into cells (e.g., verapamil present at about 2 μM to about 25 μM); nitroglycerin (e.g., about 0.05 g/L to about 0.2 g/L); an anticoagulant, in one embodiment, present in an amount sufficient to help prevent clotting of residual blood (e.g., heparin or hirudin present at a concentration of about 1000 units/1 to about 100,000 units/1); or an amiloride containing compound (e.g., amiloride, ethyl isopropyl amiloride, hexamethylene amiloride, dimethyl amiloride or isobutyl amiloride present at about 1.0 μM to about 5 μM).

5.5.2. Collection and Handling of Placenta

Generally, a human placenta is recovered shortly after its expulsion after birth. In one embodiment, the placenta is recovered from a patient after informed consent and after a complete medical history of the patient is taken and is associated with the placenta. In one embodiment, the medical history continues after delivery.

Prior to recovery of perfusate, the umbilical cord blood and placental blood are removed. In certain embodiments, after delivery, the cord blood in the placenta is recovered. The placenta can be subjected to a conventional cord blood recovery process. Typically a needle or cannula is used, with the aid of gravity, to exsanguinate the placenta (see, e.g., Anderson, U.S. Pat. No. 5,372,581; Hessel et al., U.S. Pat. No. 5,415,665). The needle or cannula is usually placed in the umbilical vein and the placenta can be gently massaged to aid in draining cord blood from the placenta. Such cord blood recovery may be performed commercially, e.g., LifeBank Inc., Cedar Knolls, N.J., ViaCord, Cord Blood Registry and CryoCell. In one embodiment, the placenta is gravity drained without further manipulation so as to minimize tissue disruption during cord blood recovery.

Typically, a placenta is transported from the delivery or birthing room to another location, e.g., a laboratory, for recovery of cord blood and collection of perfusate. The placenta can be transported in a sterile, thermally insulated transport device (maintaining the temperature of the placenta between 20-28° C.), for example, by placing the placenta, with clamped proximal umbilical cord, in a sterile zip-lock plastic bag, which is then placed in an insulated container. In another embodiment, the placenta is transported in a cord blood collection kit substantially as described in U.S. Pat. No. 7,147,626. In one embodiment, the placenta is delivered to the laboratory four to twenty-four hours following delivery. In certain embodiments, the proximal umbilical cord is clamped, for example within 4-5 cm (centimeter) of the insertion into the placental disc prior to cord blood recovery. In other embodiments, the proximal umbilical cord is clamped after cord blood recovery but prior to further processing of the placenta.

The placenta, prior to collection of the perfusate, can be stored under sterile conditions and at either room temperature or at a temperature of 5 to 25° C. (centigrade). The placenta may be stored for a period of longer than forty eight hours, or for a period of four to twenty-four hours prior to perfusing the placenta to remove any residual cord blood. The placenta can be stored in an anticoagulant solution at a temperature of 5° C. to 25° C. (centigrade). Suitable anticoagulant solutions are well known in the art. For example, a solution of heparin or warfarin sodium can be used. In one embodiment, the anticoagulant solution comprises a solution of heparin (e.g., 1% w/w in 1:1000 solution). In some embodiments, the exsanguinated placenta is stored for no more than 36 hours before placental perfusate is collected.

5.5.3. Placental Perfusion

Methods of perfusing mammalian placentae and obtaining placental perfusate are disclosed, e.g., in Hariri, U.S. Pat. Nos. 7,045,148 and 7,255,879, and in U.S. Application Publication Nos. 2009/0104164, 2007/0190042 and 20070275362, issued as U.S. Pat. No. 8,057,788, the disclosures of which are hereby incorporated by reference herein in their entireties.

Perfusate can be obtained by passage of perfusion solution, e.g., saline solution, culture medium or cell collection compositions described above, through the placental vasculature. In one embodiment, a mammalian placenta is perfused by passage of perfusion solution through either or both of the umbilical artery and umbilical vein. The flow of perfusion solution through the placenta may be accomplished using, e.g., gravity flow into the placenta. For example, the perfusion solution is forced through the placenta using a pump, e.g., a peristaltic pump. The umbilical vein can be, e.g., cannulated with a cannula, e.g., a TEFLON® or plastic cannula, that is connected to a sterile connection apparatus, such as sterile tubing. The sterile connection apparatus is connected to a perfusion manifold.

In preparation for perfusion, the placenta can be oriented in such a manner that the umbilical artery and umbilical vein are located at the highest point of the placenta. The placenta can be perfused by passage of a perfusion solution through the placental vasculature, or through the placental vasculature and surrounding tissue. In one embodiment, the umbilical artery and the umbilical vein are connected simultaneously to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. In another embodiment, the perfusion solution is passed through the umbilical veins and collected from the umbilical artery, or is passed through the umbilical artery and collected from the umbilical veins, that is, is passed through only the placental vasculature (fetal tissue).

In one embodiment, for example, the umbilical artery and the umbilical vein are connected simultaneously, e.g., to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. Placental cells that are collected by this method, which can be referred to as a “pan” method, are typically a mixture of fetal and maternal cells.

In another embodiment, the perfusion solution is passed through the umbilical veins and collected from the umbilical artery, or is passed through the umbilical artery and collected from the umbilical veins. Placental cells collected by this method, which can be referred to as a “closed circuit” method, are typically almost exclusively fetal.

The closed circuit perfusion method can, in one embodiment, be performed as follows. A post-partum placenta is obtained within about 48 hours after birth. The umbilical cord is clamped and cut above the clamp. The umbilical cord can be discarded, or can processed to recover, e.g., umbilical cord stem cells, and/or to process the umbilical cord membrane for the production of a biomaterial. The amniotic membrane can be retained during perfusion, or can be separated from the chorion, e.g., using blunt dissection with the fingers. If the amniotic membrane is separated from the chorion prior to perfusion, it can be, e.g., discarded, or processed, e.g., to obtain stem cells by enzymatic digestion, or to produce, e.g., an amniotic membrane biomaterial, e.g., the biomaterial described in U.S. Application Publication No. 2004/0048796. After cleaning the placenta of all visible blood clots and residual blood, e.g., using sterile gauze, the umbilical cord vessels are exposed, e.g., by partially cutting the umbilical cord membrane to expose a cross-section of the cord. The vessels are identified, and opened, e.g., by advancing a closed alligator clamp through the cut end of each vessel. The apparatus, e.g., plastic tubing connected to a perfusion device or peristaltic pump, is then inserted into each of the placental arteries. The pump can be any pump suitable for the purpose, e.g., a peristaltic pump. Plastic tubing, connected to a sterile collection reservoir, e.g., a blood bag such as a 250 mL collection bag, is then inserted into the placental vein. Alternatively, the tubing connected to the pump is inserted into the placental vein, and tubes to a collection reservoir(s) are inserted into one or both of the placental arteries. The placenta is then perfused with a volume of perfusion solution, e.g., about 750 ml of perfusion solution. Cells in the perfusate are then collected, e.g., by centrifugation.

In one embodiment, the proximal umbilical cord is clamped during perfusion, and, more specifically, can be clamped within 4-5 cm (centimeter) of the cord's insertion into the placental disc.

The first collection of perfusion fluid from a mammalian placenta during the exsanguination process is generally colored with residual red blood cells of the cord blood and/or placental blood. The perfusion fluid becomes more colorless as perfusion proceeds and the residual cord blood cells are washed out of the placenta. Generally from 30 to 100 mL of perfusion fluid is adequate to initially flush blood from the placenta, but more or less perfusion fluid may be used depending on the observed results.

In certain embodiments, cord blood is removed from the placenta prior to perfusion (e.g., by gravity drainage), but the placenta is not flushed (e.g., perfused) with solution to remove residual blood. In certain embodiments, cord blood is removed from the placenta prior to perfusion (e.g., by gravity drainage), and the placenta is flushed (e.g., perfused) with solution to remove residual blood.

The volume of perfusion liquid used to perfuse the placenta may vary depending upon the number of placental cells to be collected, the size of the placenta, the number of collections to be made from a single placenta, etc. In various embodiments, the volume of perfusion liquid may be from 50 mL to 5000 mL, 50 mL to 4000 mL, 50 mL to 3000 mL, 100 mL to 2000 mL, 250 mL to 2000 mL, 500 mL to 2000 mL, or 750 mL to 2000 mL. Typically, the placenta is perfused with 700-800 mL of perfusion liquid following exsanguination.

The placenta can be perfused a plurality of times over the course of several hours or several days. Where the placenta is to be perfused a plurality of times, it may be maintained or cultured under aseptic conditions in a container or other suitable vessel, and perfused with a cell collection composition, or a standard perfusion solution (e.g., a normal saline solution such as phosphate buffered saline (“PBS”) with or without an anticoagulant (e.g., heparin, warfarin sodium, coumarin, bishydroxycoumarin), and/or with or without an antimicrobial agent (e.g., β-mercaptoethanol (0.1 mM); antibiotics such as streptomycin (e.g., at 40-100 μg/ml), penicillin (e.g., at 40 U/ml), amphotericin B (e.g., at 0.5 μg/ml). In one embodiment, an isolated placenta is maintained or cultured for a period of time without collecting the perfusate, such that the placenta is maintained or cultured for 1, 2, 3, 4, 5, 6, 7, 8, 9, 10, 11, 12, 13, 14, 15, 16, 17, 18, 19, 20, 21, 22, 23, or 24 hours, or 2 or 3 or more days before perfusion and collection of perfusate. The perfused placenta can be maintained for one or more additional time(s), e.g., 1, 2, 3, 4, 5, 6, 7, 8, 9, 10, 11, 12, 13, 14, 15, 16, 17, 18, 19, 20, 21, 22, 23, 24 or more hours, and perfused a second time with, e.g., 700-800 mL perfusion fluid. The placenta can be perfused 1, 2, 3, 4, 5 or more times, for example, once every 1, 2, 3, 4, 5 or 6 hours. In one embodiment, perfusion of the placenta and collection of perfusion solution, e.g., placental cell collection composition, is repeated until the number of recovered nucleated cells falls below 100 cells/ml. The perfusates at different time points can be further processed individually to recover time-dependent populations of cells, e.g., total nucleated cells. Perfusates from different time points can also be pooled.

5.5.4. Placental Perfusate and Placental Perfusate Cells

Typically, placental perfusate from a single placental perfusion comprises about 100 million to about 500 million nucleated cells, including hematopoietic cells from which NK cells and/or ILC3 cells, e.g., NK cells and/or ILC3 cells produced according to the three-stage method described herein, may be produced by the method disclosed herein. In certain embodiments, the placental perfusate or perfusate cells comprise CD34⁺ cells, e.g., hematopoietic stem or progenitor cells. Such cells can, in a more specific embodiment, comprise CD34⁺CD45⁻ stem or progenitor cells, CD34⁺CD45⁺ stem or progenitor cells, or the like. In certain embodiments, the perfusate or perfusate cells are cryopreserved prior to isolation of hematopoietic cells therefrom. In certain other embodiments, the placental perfusate comprises, or the perfusate cells comprise, only fetal cells, or a combination of fetal cells and maternal cells.

5.6. NK Cells

5.6.1. NK Cells Produced by Three-Stage Method

In another embodiment, provided herein is an isolated NK cell population, wherein said NK cells are produced according to the three-stage method described above.

In one embodiment, provided herein is an isolated NK cell population produced by a three-stage method described herein, wherein said NK cell population comprises a greater percentage of CD3−CD56+ cells than an NK progenitor cell population produced by a three-stage method described herein, e.g., an NK progenitor cell population produced by the same three-stage method with the exception that the third culture step used to produce the NK progenitor cell population was of shorter duration than the third culture step used to produce the NK cell population. In a specific embodiment, said NK cell population comprises about 70% or more, in some embodiments, 75%, 80%, 85%, 90%, 95%, 98%, or 99% CD3−CD56+ cells. In another specific embodiment, said NK cell population comprises no less than 80%, 85%, 90%, 95%, 98%, or 99% CD3−CD56+ cells. In another specific embodiment, said NK cell population comprises between 70%-75%, 75%-80%, 80%-85%, 85%-90%, 90%-95%, or 95%-99% CD3−CD56+ cells.

In certain embodiments, said CD3⁻CD56⁺ cells in said NK cell population comprises CD3⁻CD56⁺ cells that are additionally NKp46⁺. In certain embodiments, said CD3⁻CD56+ cells in said NK cell population comprises CD3⁻CD56⁺ cells that are additionally CD16-. In certain embodiments, said CD3⁻CD56⁺ cells in said NK cell population comprises CD3⁻CD56⁺ cells that are additionally CD16+. In certain embodiments, said CD3⁻CD56⁺ cells in said NK cell population comprises CD3⁻CD56⁺ cells that are additionally CD94−. In certain embodiments, said CD3⁻CD56⁺ cells in said NK cell population comprises CD3⁻CD56⁺ cells that are additionally CD94+. In certain embodiments, said CD3⁻CD56⁺ cells in said NK cell population comprises CD3⁻CD56⁺ cells that are additionally CD11a⁺. In certain embodiments, said CD3⁻CD56⁺ cells in said NK cell population comprises CD3⁻CD56⁺ cells that are additionally NKp30⁺. In certain embodiments, said CD3⁻CD56⁺ cells in said NK cell population comprises CD3⁻CD56⁺ cells that are additionally CD161⁺. In certain embodiments, said CD3⁻CD56⁺ cells in said NK cell population comprises CD3⁻CD56⁺ cells that are additionally DNAM-1⁺. In certain embodiments, said CD3⁻CD56⁺ cells in said NK cell population comprises CD3⁻CD56⁺ cells that are additionally T-bet⁺.

In one embodiment, an NK cell population produced by a three-stage method described herein comprises cells which are CD117+. In one embodiment, an NK cell population produced by a three-stage method described herein comprises cells which are NKG2D+. In one embodiment, an NK cell population produced by a three-stage method described herein comprises cells which are NKp44+. In one embodiment, an NK cell population produced by a three-stage method described herein comprises cells which are CD244+. In one embodiment, an NK cell population produced by a three-stage method described herein comprises cells which express perforin. In one embodiment, an NK cell population produced by a three-stage method described herein comprises cells which express EOMES. In one embodiment, an NK cell population produced by a three-stage method described herein comprises cells which express granzyme B. In one embodiment, an NK cell population produced by a three-stage method described herein comprises cells which secrete IFNγ, GM-CSF and/or TNFα.

5.7. ILC3 Cells

5.7.1. ILC3 Cells Produced by Three-Stage Method

In another embodiment, provided herein is an isolated ILC3 cell population, wherein said ILC3 cells are produced according to the three-stage method described above.

In one embodiment, provided herein is an isolated ILC3 cell population produced by a three-stage method described herein, wherein said ILC3 cell population comprises a greater percentage of CD3−CD56+ cells than an ILC3 progenitor cell population produced by a three-stage method described herein, e.g., an ILC3 progenitor cell population produced by the same three-stage method with the exception that the third culture step used to produce the ILC3 progenitor cell population was of shorter duration than the third culture step used to produce the ILC3 cell population. In a specific embodiment, said ILC3 cell population comprises about 70% or more, in some embodiments, 75%, 80%, 85%, 90%, 95%, 98%, or 99% CD3−CD56+ cells. In another specific embodiment, said ILC3 cell population comprises no less than 80%, 85%, 90%, 95%, 98%, or 99% CD3−CD56+ cells. In another specific embodiment, said ILC3 cell population comprises between 70%-75%, 75%-80%, 80%-85%, 85%-90%, 90%-95%, or 95%-99% CD3−CD56+ cells.

In certain embodiments, said CD3⁻CD56⁺ cells in said ILC3 cell population comprises CD3⁻CD56⁺ cells that are additionally NKp46⁻. In certain embodiments, said CD3⁻CD56⁺ cells in said ILC3 cell population comprises CD3⁻CD56⁺ cells that are additionally CD16−. In certain embodiments, said CD3⁻CD56⁺ cells in said ILC3 cell population comprises CD3⁻CD56⁺ cells that are additionally IL1R1+. In certain embodiments, said CD3⁻CD56⁺ cells in said ILC3 cell population comprises CD3⁻CD56⁺ cells that are additionally CD94−. In certain embodiments, said CD3⁻CD56⁺ cells in said ILC3 cell population comprises CD3⁻CD56⁺ cells that are additionally RORγt+. In certain embodiments, said CD3⁻CD56⁺ cells in said ILC3 cell population comprises CD3⁻CD56⁺ cells that are additionally CD11a⁻. In certain embodiments, said CD3⁻CD56⁺ cells in said ILC3 cell population comprises CD3⁻CD56⁺ cells that are additionally T-bet+.

In one embodiment, an ILC3 cell population produced by a three-stage method described herein comprises cells which are CD117+. In one embodiment, an ILC3 cell population produced by a three-stage method described herein comprises cells which are NKG2D⁻. In one embodiment, an ILC3 cell population produced by a three-stage method described herein comprises cells which are NKp30⁻. In one embodiment, an ILC3 cell population produced by a three-stage method described herein comprises cells which are CD244+. In one embodiment, an ILC3 cell population produced by a three-stage method described herein comprises cells which are DNAM-1+. In one embodiment, an ILC3 cell population produced by a three-stage method described herein comprises cells which express AHR. In one embodiment, an ILC3 cell population produced by a three-stage method described herein comprises cells which do not express perforin. In one embodiment, an ILC3 cell population produced by a three-stage method described herein comprises cells which do not express EOMES. In one embodiment, an ILC3 cell population produced by a three-stage method described herein comprises cells which do not express granzyme B. In one embodiment, an ILC3 cell population produced by a three-stage method described herein comprises cells which secrete IL-22 and/or IL-8.

In certain aspects, cell populations produced by the three-stage method described herein comprise CD11a+ cells and CD11a− cells in a ratio of 50:1, 40:1, 30:1, 20:1, 10:1, 5:1, 4:1, 3:1, 2:1, 1:1, 1:2, 1:3, 1:4, 1:5, 1:10, 1:20, 1:30, 1:40, or 1:50. In certain aspects, a population of cells described herein comprises CD11a+ cells and CD11a− cells in a ratio of 50:1. In certain aspects, a population of cells described herein comprises CD11a+ cells and CD11a− cells in a ratio of 20:1. In certain aspects, a population of cells described herein comprises CD11a+ cells and CD11a− cells in a ratio of 10:1. In certain aspects, a population of cells described herein comprises CD11a+ cells and CD11a− cells in a ratio of 5:1. In certain aspects, a population of cells described herein comprises CD11a+ cells and CD11a− cells in a ratio of 1:1. In certain aspects, a population of cells described herein comprises CD11a+ cells and CD11a− cells in a ratio of 1:5. In certain aspects, a population of cells described herein comprises CD11a+ cells and CD11a− cells in a ratio of 1:10. In certain aspects, a population of cells described herein comprises CD11a+ cells and CD11a− cells in a ratio of 1:20. In certain aspects, a population of cells described herein comprises CD11a+ cells and CD11a− cells in a ratio of 1:50.

In certain aspects, cell populations described herein are produced by combining the CD11a+ cells with the CD11a− cells in a ratio of 50:1, 40:1, 30:1, 20:1, 10:1, 5:1, 4:1, 3:1, 2:1, 1:1, 1:2, 1:3, 1:4, 1:5, 1:10, 1:20, 1:30, 1:40, or 1:50 to produce a combined population of cells. In certain aspects, a combined population of cells described herein comprises CD11a+ cells and CD11a− cells combined in a ratio of 50:1. In certain aspects, a combined population of cells described herein comprises CD11a+ cells and CD11a− cells combined in a ratio of 20:1. In certain aspects, a combined population of cells described herein comprises CD11a+ cells and CD11a− cells combined in a ratio of 10:1. In certain aspects, a combined population of cells described herein comprises CD11a+ cells and CD11a− cells combined in a ratio of 5:1. In certain aspects, a combined population of cells described herein comprises CD11a+ cells and CD11a− cells combined in a ratio of 1:1. In certain aspects, a combined population of cells described herein comprises CD11a+ cells and CD11a− cells combined in a ratio of 1:5. In certain aspects, a combined population of cells described herein comprises CD11a+ cells and CD11a− cells combined in a ratio of 1:10. In certain aspects, a combined population of cells described herein comprises CD11a+ cells and CD11a− cells combined in a ratio of 1:20. In certain aspects, a combined population of cells described herein comprises CD11a+ cells and CD11a− cells combined in a ratio of 1:50.

In certain aspects, cell populations produced by the three-stage method described herein comprise NK cells and ILC3 cells in a ratio of 50:1, 40:1, 30:1, 20:1, 10:1, 5:1, 4:1, 3:1, 2:1, 1:1, 1:2, 1:3, 1:4, 1:5, 1:10, 1:20, 1:30, 1:40, or 1:50. In certain aspects, a population of cells described herein comprises NK cells and ILC3 cells in a ratio of 50:1. In certain aspects, a population of cells described herein comprises NK cells and ILC3 cells in a ratio of 20:1. In certain aspects, a population of cells described herein comprises NK cells and ILC3 cells in a ratio of 10:1. In certain aspects, a population of cells described herein comprises NK cells and ILC3 cells in a ratio of 5:1. In certain aspects, a population of cells described herein comprises NK cells and ILC3 cells in a ratio of 1:1. In certain aspects, a population of cells described herein comprises NK cells and ILC3 cells in a ratio of 1:5. In certain aspects, a population of cells described herein comprises NK cells and ILC3 cells in a ratio of 1:10. In certain aspects, a population of cells described herein comprises NK cells and ILC3 cells in a ratio of 1:20. In certain aspects, a population of cells described herein comprises NK cells and ILC3 cells in a ratio of 1:50.

In certain aspects, cell populations described herein are produced by combining the NK cells with the ILC3 cells in a ratio of 50:1, 40:1, 30:1, 20:1, 10:1, 5:1, 4:1, 3:1, 2:1, 1:1, 1:2, 1:3, 1:4, 1:5, 1:10, 1:20, 1:30, 1:40, or 1:50 to produce a combined population of cells. In certain aspects, a combined population of cells described herein comprises NK cells and ILC3 cells combined in a ratio of 50:1. In certain aspects, a combined population of cells described herein comprises NK cells and ILC3 cells combined in a ratio of 20:1. In certain aspects, a combined population of cells described herein comprises NK cells and ILC3 cells combined in a ratio of 10:1. In certain aspects, a combined population of cells described herein comprises NK cells and ILC3 cells combined in a ratio of 5:1. In certain aspects, a combined population of cells described herein comprises NK cells and ILC3 cells combined in a ratio of 1:1. In certain aspects, a combined population of cells described herein comprises NK cells and ILC3 cells combined in a ratio of 1:5. In certain aspects, a combined population of cells described herein comprises NK cells and ILC3 cells combined in a ratio of 1:10. In certain aspects, a combined population of cells described herein comprises NK cells and ILC3 cells combined in a ratio of 1:20. In certain aspects, a combined population of cells described herein comprises NK cells and ILC3 cells combined in a ratio of 1:50.

5.8. Compositions Comprising NK Cells and/or ILC3 Cells

5.8.1. NK Cells and/or ILC3 Cells Produced Using the Three-Stage Method

In some embodiments, provided herein is a composition, e.g., a pharmaceutical composition, comprising an isolated NK cell and/or ILC3 cell population produced using the three-stage method described herein. In a specific embodiment, said isolated NK cell and/or ILC3 cell population is produced from hematopoietic cells, e.g., hematopoietic stem or progenitor cells isolated from placental perfusate, umbilical cord blood, and/or peripheral blood. In another specific embodiment, said isolated NK cell and/or ILC3 cell population comprises at least 50% of cells in the composition. In another specific embodiment, said isolated NK cell and/or ILC3 cell population, e.g., CD3⁻CD56⁺ cells, comprises at least 80%, 85%, 90%. 95%, 98% or 99% of cells in the composition. In certain embodiments, no more than 5%, 10%, 15%, 20%, 25%, 30%, 35%, or 40% of the cells in said isolated NK cell and/or ILC3 cell population are CD3⁻CD56⁺ cells. In certain embodiments, said CD3⁻CD56⁺ cells are CD16⁻.

NK cell and/or ILC3 cell populations produced using the three-stage method described herein, can be formulated into pharmaceutical compositions for use in vivo. Such pharmaceutical compositions comprise a population of NK cells and/or ILC3 cells in a pharmaceutically-acceptable carrier, e.g., a saline solution or other accepted physiologically-acceptable solution for in vivo administration. Pharmaceutical compositions of the invention can comprise any of the NK cell and/or ILC3 cell populations described elsewhere herein.

The pharmaceutical compositions of the invention comprise populations of cells that comprise 50% viable cells or more (that is, at least 50% of the cells in the population are functional or living). Preferably, at least 60% of the cells in the population are viable. More preferably, at least 70%, 80%, 90%, 95%, or 99% of the cells in the population in the pharmaceutical composition are viable.

The pharmaceutical compositions of the invention can comprise one or more compounds that, e.g., facilitate engraftment; stabilizers such as albumin, dextran 40, gelatin, hydroxyethyl starch, and the like.

When formulated as an injectable solution, in one embodiment, the pharmaceutical composition of the invention comprises about 1.25% HSA and about 2.5% dextran. Other injectable formulations, suitable for the administration of cellular products, may be used.

In one embodiment, the compositions, e.g., pharmaceutical compositions, provided herein are suitable for systemic or local administration. In specific embodiments, the compositions, e.g., pharmaceutical compositions, provided herein are suitable for parenteral administration. In specific embodiments, the compositions, e.g., pharmaceutical compositions, provided herein are suitable for injection, infusion, intravenous (IV) administration, intrafemoral administration, or intratumor administration. In specific embodiments, the compositions, e.g., pharmaceutical compositions, provided herein are suitable for administration via a device, a matrix, or a scaffold. In specific embodiments, the compositions, e.g., pharmaceutical compositions provided herein are suitable for injection. In specific embodiments, the compositions, e.g., pharmaceutical compositions, provided herein are suitable for administration via a catheter. In specific embodiments, the compositions, e.g., pharmaceutical compositions, provided herein are suitable for local injection. In more specific embodiments, the compositions, e.g., pharmaceutical compositions, provided herein are suitable for local injection directly into a solid tumor (e.g., a sarcoma). In specific embodiments, the compositions, e.g., pharmaceutical compositions, provided herein are suitable for injection by syringe. In specific embodiments, the compositions, e.g., pharmaceutical compositions, provided herein are suitable for administration via guided delivery. In specific embodiments, the compositions, e.g., pharmaceutical compositions, provided herein are suitable for injection aided by laparoscopy, endoscopy, ultrasound, computed tomography, magnetic resonance, or radiology.

In certain embodiments, the compositions, e.g., pharmaceutical compositions provided herein, comprising NK cells and/or ILC3 cells produced using the methods described herein, are provided as pharmaceutical grade administrable units. Such units can be provided in discrete volumes, e.g., 15 mL, 20 mL, 25 mL, 30 nL. 35 mL, 40 mL, 45 mL, 50 mL, 55 mL, 60 mL, 65 mL, 70 mL, 75 mL, 80 mL, 85 mL, 90 mL, 95 mL, 100 mL, 150 mL, 200 mL, 250 mL, 300 mL, 350 mL, 400 mL, 450 mL, 500 mL, or the like. Such units can be provided so as to contain a specified number of cells, e.g., NK cells and/or ILC3 cells, e.g., 1×10⁴, 5×10⁴, 1×10⁵, 5×10⁵, 1×10⁶, 5×10⁶, 1×10⁷, 5×10⁷, 1×10⁸, 5×10⁸ or more cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more cells per unit. In specific embodiments, the units can comprise about, at least about, or at most about 1×10⁴, 5×10⁴, 1×10⁵, 5×10⁵, 1×10⁶, 5×10⁶ or more NK cells and/or ILC3 cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more cells per unit. Such units can be provided to contain specified numbers of NK cells and/or ILC3 cells or NK cell and/or ILC3 cell populations and/or any of the other cells. In specific embodiments, the NK cells and ILC3 cells are present in ratios provided herein.

In another specific embodiment, said isolated NK cells and/or ILC3 cells in said composition are from a single individual. In a more specific embodiment, said isolated NK cells and/or ILC3 cells comprise NK cells and/or ILC3 cells from at least two different individuals. In another specific embodiment, said isolated NK cells and/or ILC3 cells in said composition are from a different individual than the individual for whom treatment with the NK cells and/or ILC3 cells is intended. In another specific embodiment, said NK cells have been contacted or brought into proximity with an immunomodulatory compound or thalidomide in an amount and for a time sufficient for said NK cells to express detectably more granzyme B or perforin than an equivalent number of natural killer cells, i.e. NK cells not contacted or brought into proximity with said immunomodulatory compound or thalidomide. In another specific embodiment, said composition additionally comprises an immunomodulatory compound or thalidomide. In certain embodiments, the immunomodulatory compound is a compound described below. See, e.g., U.S. Pat. No. 7,498,171, the disclosure of which is hereby incorporated by reference in its entirety. In certain embodiments, the immunomodulatory compound is an amino-substituted isoindoline. In one embodiment, the immunomodulatory compound is 3-(4-amino-1-oxo-1,3-dihydroisoindol-2-yl)-piperidine-2,6-dione; 3-(4′aminoisolindoline-1′-one)-1-piperidine-2,6-dione; 4-(amino)-2-(2,6-dioxo(3-piperidyl))-isoindoline-1,3-dione; or 4-Amino-2-(2,6-dioxopiperidin-3-yl)isoindole-1,3-dione. In another embodiment, the immunomodulatory compound is pomalidomide, or lenalidomide. In another embodiment, said immunomodulatory compound is a compound having the structure

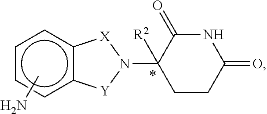

wherein one of X and Y is C═O, the other of X and Y is C═O or CH₂, and R² is hydrogen or lower alkyl, or a pharmaceutically acceptable salt, hydrate, solvate, clathrate, enantiomer, diastereomer, racemate, or mixture of stereoisomers thereof. In another embodiment, said immunomodulatory compound is a compound having the structure

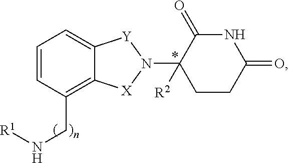

wherein one of X and Y is C═O and the other is CH₂ or C═O;

R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;

R² is H, F, benzyl, (C₁-C₈)alkyl, (C₂-C₈)alkenyl, or (C₂-C₈)alkynyl;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl;

each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;

n is 0 or 1; and

* represents a chiral-carbon center;

or a pharmaceutically acceptable salt, hydrate, solvate, clathrate, enantiomer, diastereomer, racemate, or mixture of stereoisomers thereof. In another embodiment, said immunomodulatory compound is a compound having the structure

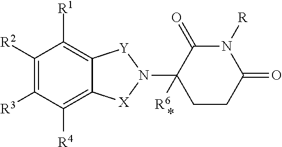

wherein:

one of X and Y is C═O and the other is CH₂ or C═O;

R is H or CH₂OCOR′;

(i) each of R′, R², R³, or R⁴, independently of the others, is halo, alkyl of 1 to 4 carbon atoms, or alkoxy of 1 to 4 carbon atoms or (ii) one of R′, R², R³, or R⁴ is nitro or —NHR⁵ and the remaining of R′, R², R³, or R⁴ are hydrogen;

R⁵ is hydrogen or alkyl of 1 to 8 carbons

R⁶ hydrogen, alkyl of 1 to 8 carbon atoms, benzo, chloro, or fluoro;

R′ is R⁷—CHR¹⁰—N(R⁸R⁹);

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;

R¹⁰ is hydrogen, alkyl of to 8 carbon atoms, or phenyl; and

* represents a chiral-carbon center;

or a pharmaceutically acceptable salt, hydrate, solvate, clathrate, enantiomer, diastereomer, racemate, or mixture of stereoisomers thereof.

In another specific embodiment, the composition additionally comprises one or more anticancer compounds, e.g., one or more of the anticancer compounds described below.

In a more specific embodiment, the composition comprises NK cells and/or ILC3 cells from another source, or made by another method. In a specific embodiment, said other source is placental blood and/or umbilical cord blood. In another specific embodiment, said other source is peripheral blood. In more specific embodiments, the NK cell and/or ILC3 cell population in said composition is combined with NK cells and/or ILC3 cells from another source, or made by another method in a ratio of about 100:1, 95:5, 90:10, 85:15, 80:20, 75:25, 70:30, 65:35, 60:40, 55:45: 50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like.

In another specific embodiment, the composition comprises an NK cell and/or ILC3 cell population produced using the three-stage method described herein and either isolated placental perfusate or isolated placental perfusate cells. In a more specific embodiment, said placental perfusate is from the same individual as said NK cell and/or ILC3 cell population. In another more specific embodiment, said placental perfusate comprises placental perfusate from a different individual than said NK cell and/or ILC3 cell population. In another specific embodiment, all, or substantially all (e.g., greater than 90%, 95%, 98% or 99%) of cells in said placental perfusate are fetal cells. In another specific embodiment, the placental perfusate or placental perfusate cells, comprise fetal and maternal cells. In a more specific embodiment, the fetal cells in said placental perfusate comprise less than about 90%, 80%, 70%, 60% or 50% of the cells in said perfusate. In another specific embodiment, said perfusate is obtained by passage of a 0.9% NaCl solution through the placental vasculature. In another specific embodiment, said perfusate comprises a culture medium. In another specific embodiment, said perfusate has been treated to remove erythrocytes. In another specific embodiment, said composition comprises an immunomodulatory compound, e.g., an immunomodulatory compound described below, e.g., an amino-substituted isoindoline compound. In another specific embodiment, the composition additionally comprises one or more anticancer compounds, e.g., one or more of the anticancer compounds described below.

In another specific embodiment, the composition comprises an NK cell and/or ILC3 cell population and placental perfusate cells. In a more specific embodiment, said placental perfusate cells are from the same individual as said NK cell and/or ILC3 cell population. In another more specific embodiment, said placental perfusate cells are from a different individual than said NK cell and/or ILC3 cell population. In another specific embodiment, the composition comprises isolated placental perfusate and isolated placental perfusate cells, wherein said isolated perfusate and said isolated placental perfusate cells are from different individuals. In another more specific embodiment of any of the above embodiments comprising placental perfusate, said placental perfusate comprises placental perfusate from at least two individuals. In another more specific embodiment of any of the above embodiments comprising placental perfusate cells, said isolated placental perfusate cells are from at least two individuals. In another specific embodiment, said composition comprises an immunomodulatory compound. In another specific embodiment, the composition additionally comprises one or more anticancer compounds, e.g., one or more of the anticancer compounds described below.

### 6. KITS

Provided herein is a pharmaceutical pack or kit comprising one or more containers filled with one or more of the compositions described herein, e.g., a composition comprising NK cells and/or ILC3 cells produced by a method described herein, e.g., NK cell and/or ILC3 cell populations produced using the three-stage method described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

The kits encompassed herein can be used in accordance with the methods described herein, e.g., methods of suppressing the growth of tumor cells and/or methods of treating cancer, e.g., hematologic cancer, and/or methods of treating viral infection. In one embodiment, a kit comprises NK cells and/or ILC3 cells produced by a method described herein or a composition thereof, in one or more containers. In a specific embodiment, provided herein is a kit comprising an NK cell and/or ILC3 cell population produced by a three-stage method described herein, or a composition thereof.

### 7. EXAMPLES

### 7.1. Example 1: Three-Stage Method of Producing Natural Killer Cells from Hematopoietic Stem or Progenitor Cells

CD34⁺ cells are cultured in the following medium formulations for the indicated number of days, and aliquots of cells are taken for assessment of cell count, cell viability, characterization of natural killer cell differentiation and functional evaluation.

Stage 1 medium: 90% Stem Cell Growth Medium (SCGM) (CellGro®), 10% Human Serum-AB, supplemented with 25 ng/mL or 250 ng/mL recombinant human thrombopoietin (TPO), 25 ng/mL recombinant human Flt3L, 27 ng/mL recombinant human stem cell factor (SCF), 25 ng/mL recombinant human IL-7, 0.05 ng/mL or 0.025 ng/mL recombinant human IL-6, 0.25 ng/mL or 0.125 ng/mL recombinant human granulocyte colony-stimulating factor (G-CSF), 0.01 ng/mL or 0.025 ng/mL recombinant human granulocyte-macrophage colony-stimulating factor (GM-CSF), 0.10% gentamicin, and 1 to 10 μm StemRegenin-1 (SR-1) or other stem cell mobilizing agent.

Stage 2 medium: 90% SCGM, 10% Human Serum-AB, supplemented with 25 ng/mL recombinant human Flt3L, 27 ng/mL recombinant human SCF, 25 ng/mL recombinant human IL-7, 20 ng/mL recombinant human IL-15, 0.05 ng/mL or 0.025 ng/mL recombinant human IL-6, 0.25 ng/mL or 0.125 ng/mL recombinant human granulocyte colony-stimulating factor (G-CSF), 0.01 ng/mL or 0.025 ng/mL recombinant human granulocyte-macrophage colony-stimulating factor (GM-CSF), 0.10% gentamicin, and 1 to 10 μm SR1 or other stem cell mobilizing agent.

Stage 3 medium: 90% STEMMACS′, 10% Human Serum-AB, 0.025 mM 2-mercaptoethanol (55 mM), supplemented with 22 ng/mL recombinant human SCF, 1000 U/mL recombinant human IL-2, 20 ng/mL recombinant human IL-7, 20 ng/mL recombinant human IL-15, 0.05 ng/mL or 0.025 ng/mL recombinant human IL-6, 0.25 ng/mL or 0.125 ng/mL recombinant human granulocyte colony-stimulating factor (G-CSF), 0.01 ng/mL or 0.025 ng/mL recombinant human granulocyte-macrophage colony-stimulating factor (GM-CSF), and 0.10% gentamicin.

Cells are seeded at Day 0 at 3×10⁴ cells/mL in Stage 1 media, and cells are tested for purity by a CD34+ and CD45+ count and viability by 7AAD staining. At Day 5 cells are counted and seeded to a concentration of 1×10⁵ cells/mL with Stage 1 medium. At Day 7 cells are counted and seeded to a concentration of 1×10⁵ cells/mL with Stage 1 medium.

At Day 10, cells are counted and seeded to a concentration of 1×10⁵ cells/mL in Stage 2 medium. At Day 12, cells are counted and seeded to a concentration of 3×10⁵ cells/mL in Stage 2 medium. At Day 14, cells are counted and seeded in Stage 3 medium. Cells are maintained in Stage 3 media until day 35.

Alternatively, the following protocol is used through Day 14: Cells seeded at Day 0 at 7.5×10³ cells/mL in Stage 1 media, and cells are tested for purity by a CD34+ and CD45+ count and viability by 7AAD staining. At Day 7 cells are counted and seeded to a concentration of 3×10⁵ cells/mL with Stage 1 medium. At Day 9 cells are counted and seeded to a concentration of 3×10⁵ cells/mL with Stage 2 medium. At Day 12, cells are counted and seeded to a concentration of 3×10⁵ cells/mL in Stage 2 medium. At Day 14, cells are counted and seeded to a concentration of 3×10⁵ cells/mL in Stage 2 medium.

Seeding of cells into at passage is performed either by dilution of the culture with fresh media or by centrifugation of cells and resuspension/addition of fresh media.

For harvest, cells are spun at 400×g for seven minutes, followed by suspension of the pellet in an equal volume of Plasmalyte A. The suspension is spun at 400×g for seven minutes, and the resulting pellet is suspended in 10% HSA (w/v), 60% Plasmalyte A (v/v) at the target cell concentration. The cells are then strained through a 70 μm mesh, the final container is filled, an aliquot of the cells are tested for viability, cytotoxicity, purity, and cell count, and the remainder is packaged.

### 7.2. Example 2: Selection of Stem Cell Mobilizing Agents for the Expansion of NK Cells

The following compounds were investigated for their ability to promote the expansion of NK cell populations in vitro:
4-(2-((2-(benzo[b]thiophen-3-yl)-6-(isopropylamino)pyrimidin-4-yl)amino)ethyl)phenol) (“CRL1”)

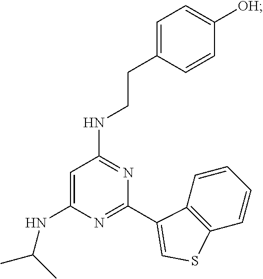 [SMILES: CC(C)Nc1cc(NCCc2ccc(O)cc2)nc(-c2csc3ccccc23)n1]

4-(2-((2-(benzo[b]thiophen-3-yl)-7-isopropylthieno[3,2-d]pyrimidin-4-yl)amino)ethyl)phenol)) (“CRL2”)

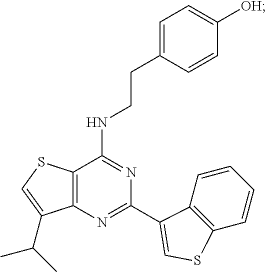 [SMILES: CC(C)c1csc2c(NCCc3ccc(O)cc3)nc(-c3csc4ccccc34)nc12]

4-(2-((2-(benzo[b]thiophen-3-yl)-7-isopropyl-6,7-dihydro-5H-pyrrolo[2,3-d]pyrimidin-4-yl)amino)ethyl)phenol (“CRL3”)

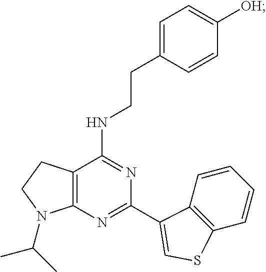 [SMILES: CC(C)N1CCc2c(NCCc3ccc(O)cc3)nc(-c3csc4ccccc34)nc21]

2-(benzo[b]thiophen-3-yl)-4-((4-hydroxyphenethyl)amino)-7-isopropyl-5,7-dihydro-6H-pyrrolo[2,3-d]pyrimidin-6-one (“CRL4”)

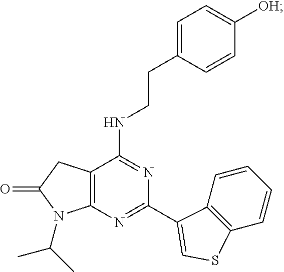 [SMILES: CC(C)N1C(=O)Cc2c(NCCc3ccc(O)cc3)nc(-c3csc4ccccc34)nc21]

3-((2-(benzo[b]thiophen-3-yl)-9-isopropyl-9H-purin-6-yl)oxy)propanamide (“CRL5”)

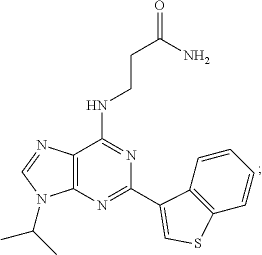 [SMILES: CC(C)n1cnc2c(NCCC(N)=O)nc(-c3csc4ccccc34)nc21]

4-(2-((2-(benzo[b]thiophen-3-yl)-8-(dimethylamino)pyrimido[5,4-d]pyrimidin-4-yl)amino)ethyl)phenol (“CRL6”)

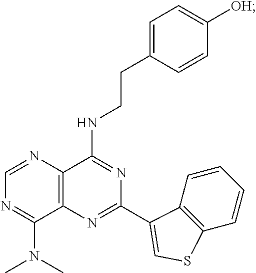 [SMILES: CN(C)c1ncnc2c(NCCc3ccc(O)cc3)nc(-c3csc4ccccc34)nc12]

5-(2-((2-(1H-indol-3-yl)ethyl)amino)-6-(sec-butylamino)pyrimidin-4-yl)nicotinonitrile (“CRL7”)

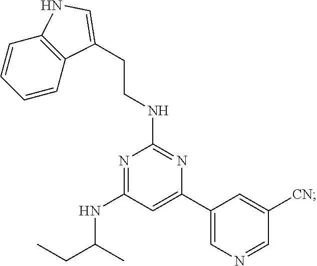 [SMILES: CCC(C)Nc1cc(-c2cncc(C#N)c2)nc(NCCc2c[nH]c3ccccc23)n1]

N-(2-(1H-indol-3-yl)ethyl)-2-methyl-6-phenylthieno[2,3-d]pyrimidin-4-amine (“CRL8”)

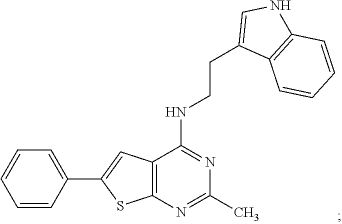 [SMILES: Cc1nc(NCCc2c[nH]c3ccccc23)c2cc(-c3ccccc3)sc2n1]

N-(2-(1H-indol-3-yl)ethyl)-6-(4-fluorophenyl)thieno[2,3-d]pyrimidin-4-amine (“CRL9”)

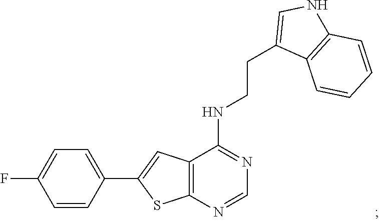 [SMILES: Fc1ccc(-c2cc3c(NCCc4c[nH]c5ccccc45)ncnc3s2)cc1]

3-(2-(benzo[b]thiophen-3-yl)-9-isopropyl-6-oxo-6,9-dihydro-1H-purin-1-yl)propanamide (“CRL10”)

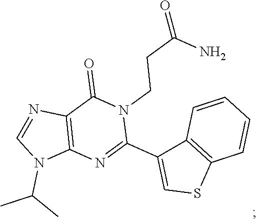 [SMILES: CC(C)n1cnc2c(=O)n(CCC(N)=O)c(-c3csc4ccccc34)nc21]

N-(2-(1H-indol-3-yl)ethyl)-2-(5-fluoropyridin-3-yl)quinazolin-4-amine (“CRL11”)

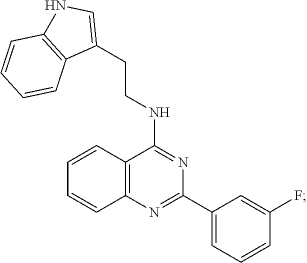 [SMILES: Fc1cccc(-c2nc(NCCc3c[nH]c4ccccc34)c3ccccc3n2)c1]

5-(4-((2-(1H-indol-3-yl)ethyl)amino)quinazolin-2-yl)nicotinonitrile (“CRL12”)

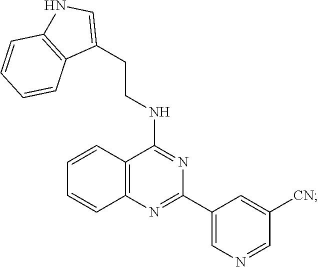 [SMILES: N#Cc1cncc(-c2nc(NCCc3c[nH]c4ccccc34)c3ccccc3n2)c1]

N⁴-(2-(1H-indol-3-yl)ethyl)-N²-(sec-butyl)quinazoline-2,4-diamine (“CRL13”)

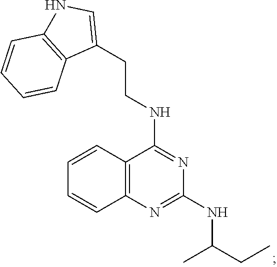 [SMILES: CCC(C)Nc1nc(NCCc2c[nH]c3ccccc23)c2ccccc2n1]

2-(benzo[b]thiophen-3-yl)-4-((4-hydroxyphenethyl)amino)-7-isopropyl-7H-pyrrolo[2,3-d]pyrimidine-5-carbonitrile (“CRL14”)

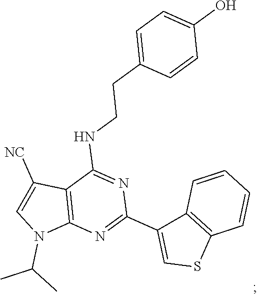 [SMILES: CC(C)n1cc(C#N)c2c(NCCc3ccc(O)cc3)nc(-c3csc4ccccc34)nc21]

N-(2-(1H-indol-3-yl)ethyl)-6-(benzo[b]thiophen-3-yl)-3-isopropylimidazo[1,5-a]pyrazin-8-amine (“CRL15”)

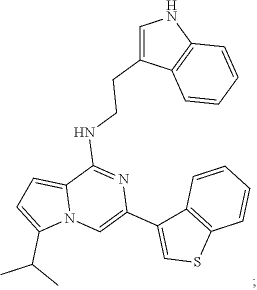 [SMILES: CC(C)c1ccc2c(NCCc3c[nH]c4ccccc34)nc(-c3csc4ccccc34)cn12]

4-(2-((6-(benzo[b]thiophen-3-yl)-3-isopropylimidazo[1,5-a]pyrazin-8-yl)amino)ethyl)phenol (“CRL16”)

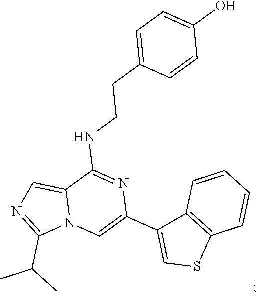 [SMILES: CC(C)c1ncc2c(NCCc3ccc(O)cc3)nc(-c3csc4ccccc34)cn12]

5-(4-((2-(1H-indol-3-yl)ethyl)amino)-7-isopropylthieno[3,2-d]pyrimidin-2-yl)nicotinonitrile (“CRL17”)

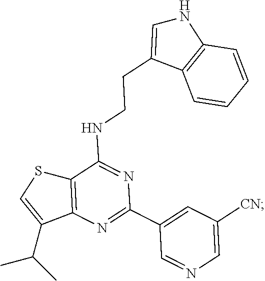 [SMILES: CC(C)c1csc2c(NCCc3c[nH]c4ccccc34)nc(-c3cncc(C#N)c3)nc12]

N-(2-(1H-indol-3-yl)ethyl)-2-(5-fluoropyridin-3-yl)-7-isopropylthieno[3,2-d]pyrimidin-4-amine (“CRL18”)

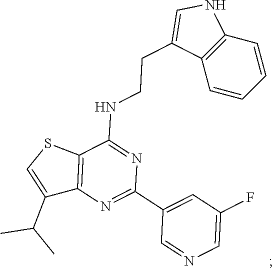 [SMILES: CC(C)c1csc2c(NCCc3c[nH]c4ccccc34)nc(-c3cncc(F)c3)nc12]

N-(2-(1H-indol-3-yl)ethyl)-2-(5-fluoropyridin-3-yl)furo[3,2-d]pyrimidin-4-amine (“CRL19”)

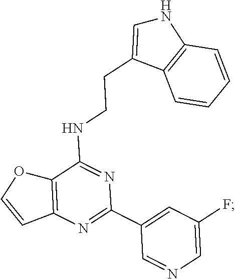 [SMILES: Fc1cncc(-c2nc(NCCc3c[nH]c4ccccc34)c3occc3n2)c1]

N-(2-(1H-indol-3-yl)ethyl)-2-(5-methylpyridin-3-yl)furo[3,2-d]pyrimidin-4-amine (“CRL20”)

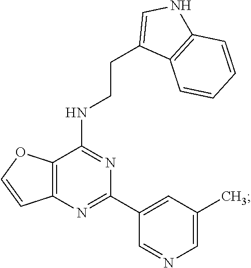 [SMILES: Cc1cncc(-c2nc(NCCc3c[nH]c4ccccc34)c3occc3n2)c1]

N-(2-(1H-indol-3-yl)ethyl)-7-isopropyl-2-(5-methylpyridin-3-yl)thieno[3,2-d]pyrimidin-4-amine (“CRL21”)

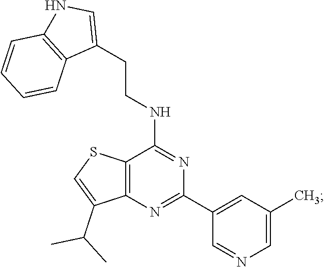 [SMILES: Cc1cncc(-c2nc(NCCc3c[nH]c4ccccc34)c3scc(C(C)C)c3n2)c1]

and
5-(4-((2-(1H-indol-3-yl)ethyl)amino)furo[3,2-d]pyrimidin-2-yl)nicotinonitrile (“CRL22”)

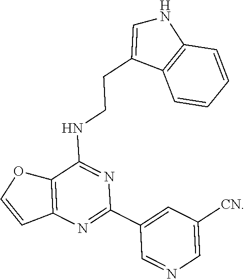 [SMILES: N#Cc1cncc(-c2nc(NCCc3c[nH]c4ccccc34)c3occc3n2)c1]

### 7.3. Example 3: Characterization of Three-Stage NK Cells

### Methods

UCB CD34+ cells were cultivated in presence of cytokines including thrombopoietin, SCF, Flt3 ligand, IL-7, IL-15 and IL-2 for 35 days to produce three-stage NK cells, as described in Example 1. Multi-color flow cytometry was used to determine the phenotypic characteristics of three-stage NK cells.

For biological testing, the compounds were provided to culture to evaluate their effects on NK cell expansion and differentiation. Specifically, donors of CD34+ cells (StemCell Technology) were thawed and expanded in vitro following NK culture protocol. During the first 14 days of the culture, each CRL compounds was dissolved in DMSO and added to the culture at 10 μM concentration. SR1 (at 10 μM) served as a positive control compound, while DMSO alone without any compound served as a negative control. At the end of the culture on Day 35, cell expansion, natural killer (NK) cell differentiation and cytotoxicity of the cells against K562 tumor cell line were characterized. Due to the large number of the compounds, the testing was performed in two experiments, CRL1-11 and CRL 12-22. The same donors were used for each experiment. Positive and negative controls were also included in both experiments.

### Results

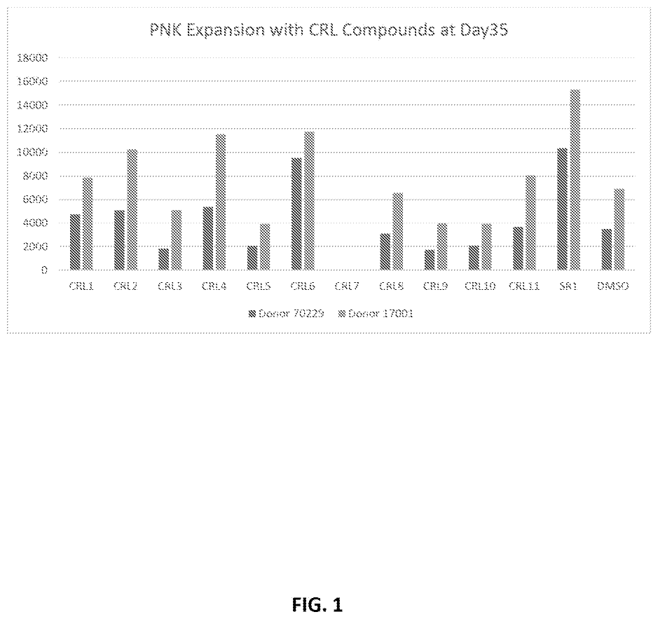

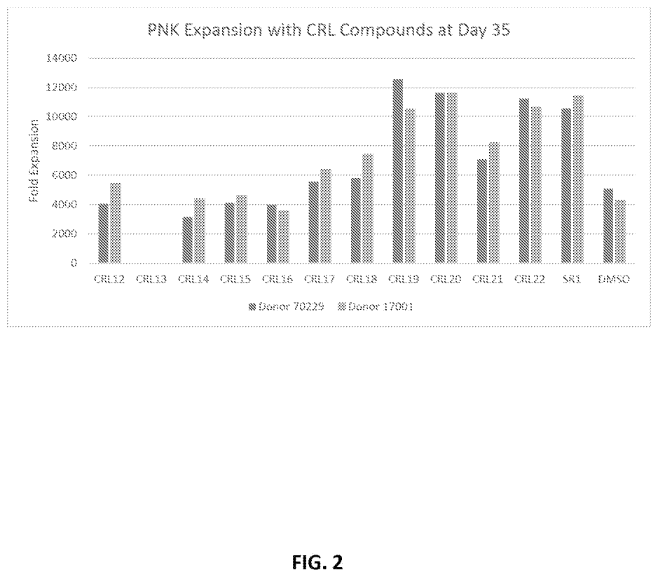

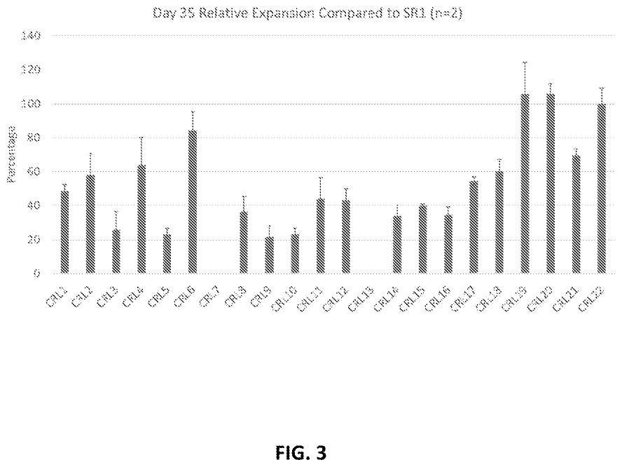

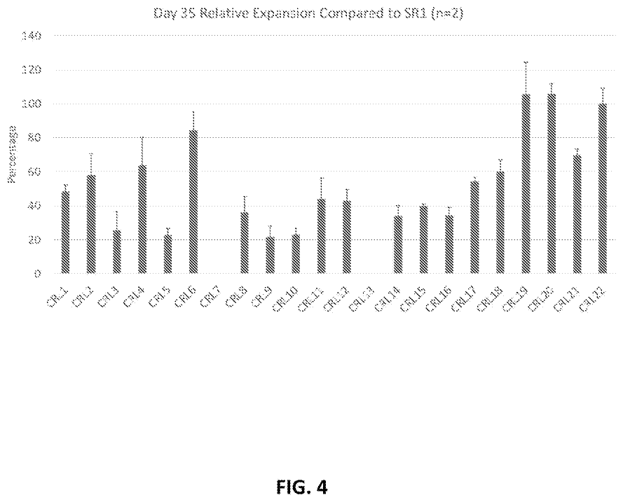

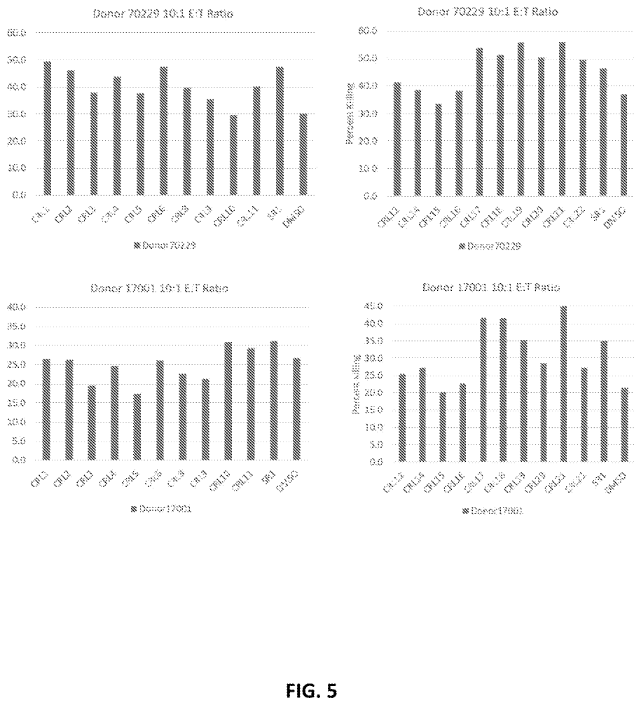

Cell expansion data showed that 20 out of the 22 compounds supported NK expansion at 10 μM concentration. Except for CRL7 and CRL13, the rest of the compounds all resulted in a NK expansion of 2,000˜15,000 fold over 35 days (FIG. 1 and FIG. 2). Among all the compounds, CRL19, 20 and 22 supported cell expansion the best, and they demonstrated a similar level of expansion compared to SR1 at Day 35 (FIG. 3). CD34 cell expansion at Day 14 of the culture showed a similar trend that most of the compounds supported CD34 cells expansion, and CRL19, 20 and 22 achieved the highest CD34 cell expansion at Day 14 (FIG. 4).

Cytotoxicity assay was run using compound cultured cells against K562 tumor cells at 10:1 effector to target ratio (FIG. 5) to evaluate cell functions. The results showed that the cells cultured with compounds killed 3060% of K562 cells at 10:1 E:T ratio, indicating that the cells present NK functions. For both donors, cells cultured with CRL17, 18, 19 and 21 demonstrated similar or greater killing activities compared to those cultured with SR1.

### Conclusions:

In summary, we found that all the compounds except CRL7 and CRL13 supported PNK-007 expansion and differentiation. Expansion with the compounds ranged from 2,000˜15,000 fold over 35 days, and the culture achieved more than 70% of NK cells. Among these compounds, CRL 19, 20 and 22 demonstrated very similar expansion, differentiation and cytotoxicity profiles as SR1 for PNK-007 culture. CRL 17, 18, and 21 resulted in slightly less expansion compared to SR1 but increased CD56+/CD11a+ subpopulation, and also increased killing activities of the cells.

### 7.4 Example 4: Further Characterization of Three-Stage NK Cells

### Methods

Cells: Frozen PBMC were acquired from Stem Cell Technologies. Peripheral blood derived NKs (PB-NK) cells were isolated from fresh blood of healthy donors using the Human NK Cell Enrichment Kit (Stem Cell Technologies) according to manufacturer's instructions. CYNK cells were generated from umbilical cord blood-derived CD34+ stem cells (Ref: Zhang et al. J Immunother Cancer. 2015). Briefly, the CD34+ cells were cultivated in the presence of cytokines including thromobopoietin, SCF, Flt3 ligand, IL-7, IL-15 and IL-2 for 35 days. PBNK and CYNK cells were cryopreserved until analysis.

Magnetic-activated cell sorting: PNK cells were stained with PE Mouse Anti-Human CD11a (BD) and CD11a+ PNK cells concentrated using anti-PE MicroBeads according to manufacturer's instructions (Miltenyi Biotec).

Single cell RNA sequencing: CYNK cells were combined with PB-NK at 1:1 ratio and gene expression analyzed on single cell level using 10× Genomics Chromium platform and Illumina sequencing. Bioinformatics analysis utilized 10× Genomics Cell Ranger analysis pipeline.

Flow Cytometry: Cryopreserved cells were rapidly thawed in a 37° C. water bath and washed once in RPMI1640+10% hiFBS (heat inactivated Fetal Bovine Serum, Gibco), followed by LIVE/DEAD™ Fixable Aqua Stain in PBS. Cells were washed with FACS buffer (PBS+2% FBS) followed by incubation in blocking solution (Brilliant Stain buffer, Mouse IgG2a isotype k control and Human BD Fc Block (all from BD)). Cells were washed with FACS buffer and incubated with fluorophore-coupled antibodies in FACS buffer for 25 min on ice. Cells were washed with FACS buffer before analysis on Fortessa X20 flow cytometer (BD).

qRT-PCR: RNA was isolated from cells using Quick-RNA Miniprep kit (Qiagen) according to the manufacturer's instructions. cDNA was synthesized using SuperScript IV Reverse Transcriptase (Thermo Fisher Scientific) in a standard reaction. RT-PCR was performed using Taqman Gene expression assays (Applied Biosystems). Expression levels were calculated relative to GAPDH (Hs02758991) using the ΔΔCt method.

### Results

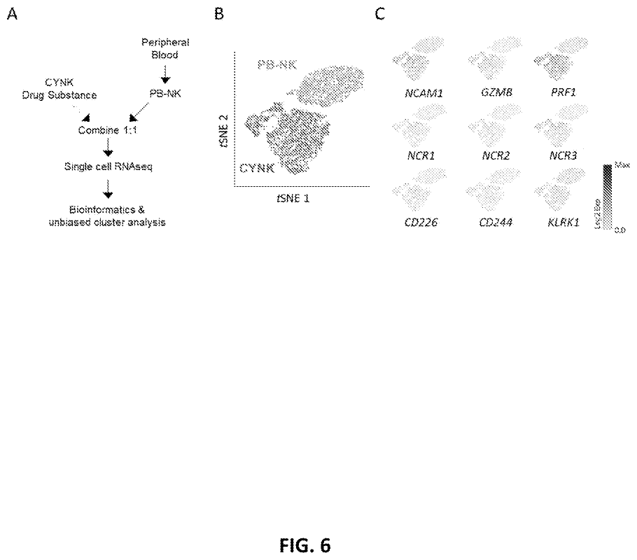

CYNK cells efficiently kill various tumor cell lines in vitro, however, the mechanisms CYNK cells use to induce cell death remains poorly understood (ref). To elucidate on the activating NK cell receptors, the intracellular signaling pathways and molecular mechanisms CYNK cells employ to carry out their functional roles, we used single-cell RNA sequencing (scRNAseq) as an unbiased approach to compare CYNK cells to peripheral blood NK cells (PB-NK) (FIG. 6A). Unbiased transcriptional clustering revealed two distinct signatures differentiating between CYNK and PB-NK cells (FIG. 6B). Tables 1 and 2 list top 50 upregulated genes per cluster in PB-NK and CYNK cells, respectively. The gene set expressed higher in PB-NK cells included genes associated with NK cell functional roles, including FGFBP2, granzymes (GZMH, GZMM), CXCR4, KLRF1, KLF2, IFNG (Table 1).
FGFBP2, encoding fibroblast growth factor-binding protein, is known to be secreted by cytotoxic lymphocytes.
Granzymes are a group of serine proteases which are stored in the cytotoxic granules of NK cells and cytotoxic T lymphocytes (ref). While GzmA and GzmB induce target cell death upon release to their cytoplasm and have been extensively studied, less is known about the functional role of GzmH, GzmK and GzmM.
CXCR4 regulates NK cell homing to bone marrow.
KLRF1 encodes NKp80, an activating C-type lectin-like immunoreceptor that is activated upon binding to activation-induced C-type lectin (AICL), inducing NK cell cytotoxicity and cytokine secretion.
Transcription factor KLF2 that regulates both NK cell proliferation and survival.
NK cell-derived IFN-γ (IFNG gene) is a key immunoregulatory factor secreted from activated NK cells that promotes adaptive immune response by modulating dendritic cell and T cell responses.

TABLE 1
Top 50 upregulated genes per PB-NK cluster.

| | | Feature | CYNK | PB-NK | PB-NK Log2 | PB-NK |
|---|---|---|---|---|---|---|
| | Feature ID | Name | Average | Average | Fold Change | P-Value |
| 1 | ENSG00000137441 | FGFBP2 | 0.099352 | 2.935962 | 4.88363 | 4.09E−78 |
| 2 | ENSG00000100450 | GZMH | 0.136708 | 2.484828 | 4.182845 | 2.49E−58 |
| 3 | ENSG00000276085 | CCL3L3 | 0.072152 | 1.251852 | 4.115143 | 2.13E−49 |
| 4 | ENSG00000197540 | GZMM | 0.134235 | 1.982728 | 3.883559 | 1.40E−50 |
| 5 | ENSG00000121966 | CXCR4 | 0.403236 | 5.935725 | 3.879087 | 9.19E−51 |
| 6 | ENSG00000169554 | ZEB2 | 0.127877 | 1.860789 | 3.861967 | 7.03E−50 |
| 7 | ENSG00000127528 | KLF2 | 0.172475 | 1.92761 | 3.481483 | 1.86E−40 |
| 8 | ENSG00000189067 | LITAF | 0.297791 | 3.231559 | 3.439184 | 1.06E−39 |
| 9 | ENSG00000069667 | RORA | 0.101913 | 1.055542 | 3.371425 | 3.26E−37 |
| 10 | ENSG00000145220 | LYAR | 0.142448 | 1.306592 | 3.196402 | 2.39E−33 |
| 11 | ENSG00000125107 | CNOT1 | 0.208595 | 1.809824 | 3.116348 | 3.39E−32 |
| 12 | ENSG00000111537 | IFNG | 0.193317 | 1.639941 | 3.083863 | 1.11E−29 |
| 13 | ENSG00000158050 | DUSP2 | 0.40774 | 3.322164 | 3.025836 | 4.12E−30 |
| 14 | ENSG00000110046 | ATG2A | 0.190226 | 1.508942 | 2.987028 | 3.39E−29 |
| 15 | ENSG00000173762 | CD7 | 0.492697 | 3.641922 | 2.885402 | 1.77E−27 |
| 16 | ENSG00000141682 | PMAIP1 | 0.252398 | 1.820017 | 2.849558 | 6.51E−26 |
| 17 | ENSG00000078304 | PPP2R5C | 0.381864 | 2.591665 | 2.762207 | 6.15E−25 |
| 18 | ENSG00000153234 | NR4A2 | 0.399174 | 2.622622 | 2.715393 | 5.59E−24 |
| 19 | ENSG00000152518 | ZFP36L2 | 0.856899 | 5.585388 | 2.703993 | 4.72E−24 |
| 20 | ENSG00000145675 | PIK3R1 | 0.325168 | 2.078618 | 2.675822 | 2.70E−23 |
| 21 | ENSG00000150045 | KLRF1 | 0.191285 | 1.177103 | 2.620822 | 4.78E−22 |
| 22 | ENSG00000255198 | SNHG9 | 0.516983 | 2.951818 | 2.512937 | 1.34E−20 |
| 23 | ENSG00000125148 | MT2A | 0.51504 | 2.913311 | 2.499426 | 9.06E−20 |
| 24 | ENSG00000116741 | RGS2 | 0.203737 | 1.147279 | 2.492865 | 1.51E−19 |
| 25 | ENSG00000153922 | CHD1 | 0.252574 | 1.350762 | 2.418474 | 9.42E−19 |
| 26 | ENSG00000120129 | DUSP1 | 2.078529 | 9.865317 | 2.24638 | 2.58E−16 |
| 27 | ENSG00000143924 | EML4 | 0.256284 | 1.150299 | 2.165756 | 7.80E−15 |
| 28 | ENSG00000128016 | ZFP36 | 2.22866 | 9.777355 | 2.132849 | 1.32E−14 |
| 29 | ENSG00000163874 | ZC3H12A | 0.261759 | 1.120475 | 2.097382 | 7.47E−14 |
| 30 | ENSG00000105993 | DNAJB6 | 0.6506 | 2.667169 | 2.035058 | 2.98E−13 |
| 31 | ENSG00000126524 | SBDS | 0.534822 | 2.185078 | 2.030148 | 3.57E−13 |
| 32 | ENSG00000125347 | IRF1 | 1.450448 | 5.812277 | 2.002193 | 7.32E−13 |
| 33 | ENSG00000157514 | TSC22D3 | 1.103379 | 4.30409 | 1.963373 | 2.57E−12 |
| 34 | ENSG00000184205 | TSPYL2 | 0.592137 | 2.247746 | 1.924086 | 1.14E−11 |
| 35 | ENSG00000146278 | PNRC1 | 1.362312 | 5.156149 | 1.919832 | 7.77E−12 |
| 36 | ENSG00000135070 | ISCA1 | 0.27898 | 1.043084 | 1.90227 | 2.06E−11 |
| 37 | ENSG00000171223 | JUNB | 4.09462 | 15.11622 | 1.883884 | 2.20E−11 |
| 38 | ENSG00000156232 | WHAMM | 0.316425 | 1.146147 | 1.856513 | 7.14E−11 |
| 39 | ENSG00000164327 | RICTOR | 0.318279 | 1.101977 | 1.791406 | 3.85E−10 |
| 40 | ENSG00000118503 | TNFAIP3 | 0.550807 | 1.902316 | 1.787777 | 3.93E−10 |
| 41 | ENSG00000120616 | EPC1 | 0.562199 | 1.846066 | 1.714953 | 2.17E−09 |
| 42 | ENSG00000167508 | MVD | 0.309448 | 1.00722 | 1.702322 | 4.11E−09 |
| 43 | ENSG00000013441 | CLK1 | 0.690164 | 2.216412 | 1.682859 | 4.62E−09 |
| 44 | ENSG00000188042 | ARL4C | 0.437325 | 1.388136 | 1.666056 | 8.18E−09 |
| 45 | ENSG00000162924 | REL | 0.553809 | 1.736208 | 1.648145 | 1.14E−08 |
| 46 | ENSG00000005483 | KMT2E | 0.79402 | 2.460289 | 1.631225 | 1.47E−08 |
| 47 | ENSG00000119801 | YPEL5 | 0.966141 | 2.98202 | 1.625617 | 1.70E−08 |
| 48 | ENSG00000123505 | AMD1 | 0.558578 | 1.664102 | 1.574595 | 6.03E−08 |
| 49 | ENSG00000159388 | BTG2 | 0.751541 | 2.22132 | 1.563151 | 7.55E−08 |
| 50 | ENSG00000010404 | IDS | 0.723193 | 2.128073 | 1.556757 | 8.48E−08 |

Top differentially expressed genes in CYNK cluster that are encode factors associated with NK cell functional role include surface receptors and co-receptors (CD96, NCR3, CD59, KLRC1), TNFSF10, immune checkpoint genes (TNFRSF18, TNFRSF4, HAVCR2), NK cell receptor adaptor molecule genes (FCER1G and LAT2) (Table 2).

TABLE 2
Top 50 upregulated genes per CYNK cluster.

| | | | | | CYNK Log2 | |
|---|---|---|---|---|---|---|
| | | Feature | PBNK | CYNK | Fold | CYNK |
| | Feature ID | Name | Average | Average | Change | P-Value |
| 1 | ENSG00000102471 | NDFIP2 | 0.077391 | 1.45981 | 4.230949 | 1.69E−22 |
| 2 | ENSG00000242258 | LINC00996 | 0.063046 | 1.183921 | 4.222944 | 5.04E−22 |
| 3 | ENSG00000172005 | MAL | 0.057005 | 1.03529 | 4.173813 | 1.35E−21 |
| 4 | ENSG00000108702 | CCL1 | 0.078524 | 1.334494 | 4.080611 | 5.11E−09 |
| 5 | ENSG00000198125 | MB | 0.10193 | 1.683947 | 4.041355 | 1.45E−20 |
| 6 | ENSG00000128040 | SPINK2 | 0.087962 | 1.233641 | 3.804242 | 7.88E−19 |
| 7 | ENSG00000166920 | C15orf48 | 0.078901 | 1.018246 | 3.683547 | 6.40E−18 |
| 8 | ENSG00000134072 | CAMK1 | 0.151762 | 1.932724 | 3.667647 | 2.13E−18 |
| 9 | ENSG00000134545 | KLRC1 | 0.509273 | 4.740451 | 3.217889 | 9.47E−16 |
| 10 | ENSG00000121858 | TNFSF10 | 0.295975 | 2.682764 | 3.178801 | 6.44E−15 |
| 11 | ENSG00000186891 | TNFRSF18 | 1.182011 | 10.09017 | 3.093605 | 6.96E−15 |
| 12 | ENSG00000008517 | IL32 | 4.345617 | 37.08234 | 3.093395 | 6.60E−15 |
| 13 | ENSG00000042493 | CAPG | 0.369213 | 3.112494 | 3.074529 | 9.91E−15 |
| 14 | ENSG00000235576 | AC092580.4 | 0.44736 | 3.660475 | 3.031759 | 2.23E−14 |
| 15 | ENSG00000163191 | S100A11 | 0.41527 | 3.364804 | 3.017543 | 2.42E−14 |
| 16 | ENSG00000186827 | TNFRSF4 | 0.135529 | 1.097816 | 3.01448 | 1.91E−13 |
| 17 | ENSG00000074800 | ENO1 | 2.166202 | 16.05066 | 2.889567 | 1.86E−13 |
| 18 | ENSG00000158869 | FCER1G | 0.734274 | 5.393877 | 2.876632 | 2.43E−13 |
| 19 | ENSG00000118971 | CCND2 | 0.457175 | 3.324621 | 2.861636 | 3.21E−13 |
| 20 | ENSG00000205426 | KRT81 | 0.169883 | 1.187806 | 2.803005 | 3.69E−12 |
| 21 | ENSG00000243927 | MRPS6 | 0.358643 | 2.29304 | 2.675597 | 6.10E−12 |
| 22 | ENSG00000182718 | ANXA2 | 0.206125 | 1.282389 | 2.635118 | 3.48E−11 |
| 23 | ENSG00000125384 | PTGER2 | 0.175546 | 1.08713 | 2.628037 | 4.29E−11 |
| 24 | ENSG00000124767 | GLO1 | 0.214053 | 1.289543 | 2.588793 | 6.50E−11 |
| 25 | ENSG00000135077 | HAVCR2 | 0.175924 | 1.031051 | 2.548543 | 1.51E−10 |
| 26 | ENSG00000103490 | PYCARD | 0.183097 | 1.070527 | 2.545209 | 1.34E−10 |
| 27 | ENSG00000086730 | LAT2 | 0.178566 | 1.04156 | 2.541707 | 1.53E−10 |
| 28 | ENSG00000141526 | SLC16A3 | 0.282006 | 1.622835 | 2.523282 | 1.73E−10 |
| 29 | ENSG00000103187 | COTL1 | 0.894342 | 5.013779 | 2.486834 | 1.45E−10 |
| 30 | ENSG00000067225 | PKM | 1.099712 | 6.145949 | 2.482453 | 1.11E−10 |
| 31 | ENSG00000177156 | TALDO1 | 0.196687 | 1.084745 | 2.46115 | 4.23E−10 |
| 32 | ENSG00000153283 | CD96 | 0.368458 | 2.029162 | 2.460314 | 1.66E−10 |
| 33 | ENSG00000204475 | NCR3 | 0.640272 | 3.472457 | 2.438804 | 2.31E−10 |
| 34 | ENSG00000170442 | KRT86 | 0.257845 | 1.372733 | 2.410873 | 1.02E−09 |
| 35 | ENSG00000117632 | STMN1 | 0.468878 | 2.413499 | 2.36315 | 1.22E−09 |
| 36 | ENSG00000227507 | LTB | 3.831437 | 19.41653 | 2.341609 | 1.09E−09 |
| 37 | ENSG00000130429 | ARPC1B | 0.570053 | 2.846585 | 2.31957 | 1.27E−09 |
| 38 | ENSG00000162704 | ARPC5 | 0.347317 | 1.717418 | 2.30484 | 1.66E−09 |
| 39 | ENSG00000088832 | FKBP1A | 0.40017 | 1.978205 | 2.304629 | 1.60E−09 |
| 40 | ENSG00000102265 | TIMP1 | 0.385447 | 1.902345 | 2.302248 | 1.96E−09 |
| 41 | ENSG00000113088 | GZMK | 0.290312 | 1.403201 | 2.27168 | 1.37E−08 |
| 42 | ENSG00000085063 | CD59 | 0.215186 | 1.035997 | 2.265377 | 7.12E−09 |
| 43 | ENSG00000102144 | PGK1 | 1.405879 | 6.735348 | 2.260328 | 2.92E−09 |
| 44 | ENSG00000148908 | RGS10 | 0.217451 | 1.014713 | 2.220352 | 1.33E−08 |
| 45 | ENSG00000196405 | EVL | 1.186164 | 5.50471 | 2.214345 | 5.41E−09 |
| 46 | ENSG00000128340 | RAC2 | 1.063092 | 4.917253 | 2.209516 | 5.72E−09 |
| 47 | ENSG00000100097 | LGALS1 | 4.427539 | 20.46621 | 2.208968 | 6.05E−09 |
| 48 | ENSG00000139626 | ITGB7 | 0.50059 | 2.285445 | 2.19016 | 8.54E−09 |
| 49 | ENSG00000196230 | TUBB | 1.062715 | 4.838214 | 2.186651 | 1.22E−08 |
| 50 | ENSG00000171314 | PGAM1 | 0.670096 | 3.046436 | 2.18433 | 8.56E−09 |

To better understand how the cytotoxic response is initiated in CYNK cells, we specifically analyzed the expression of manually chosen genes encoding well characterized proteins leading from target detection to a cytolytic response, with main focus on NK cell receptors and adaptor molecule (Table 3). Differential gene expression analysis showed high expression of the two key cytotoxic molecules perforin (PRF1) and granzyme B (GZMB) in CYNK cells. Similarly, most receptors that were differentially expressed between CYNK and PB-NK cells, with the exception of KLRF1 (encoding NKp80), were higher expressed on CYNK cells. Expression of selected NK cell effector and receptor genes is visualized on tSNE plots in FIG. 6C. Elevated expression of genes encoding components of the NK cell cytotoxic machinery correlate well with the high cytotoxic activity of CYNK cells against a broad range of target cells.

TABLE 3
Top differentially expressed genes encoding factors regulating NK cell cytolytic
function. Genes that had <1 count per cell across the entire cluster were excluded

| | | | | | | CYNK Log2 | |
|---|---|---|---|---|---|---|---|
| | | Feature | | CYNK | PBNK | Fold | CYNK |
| | Feature ID | Name | Alias | Average | Average | Change | P-Value |
| 1 | ENSG00000134545 | KLRC1 | NKG2A, CD159a | 4.740451 | 0.509273 | 3.217889 | 9.47E−16 |
| 2 | ENSG00000121858 | TNFSF10 | TRAIL | 2.682764 | 0.295975 | 3.178801 | 6.44E−15 |
| 3 | ENSG00000186891 | TNFRSF18 | GITR | 10.09017 | 1.182011 | 3.093605 | 6.96E−15 |
| 4 | ENSG00000186827 | TNFRSF4 | CD134, OX40 | 1.097816 | 0.135529 | 3.014481 | 1.91E−13 |
| 5 | ENSG00000135077 | HAVCR2 | TIM-3 | 1.031051 | 0.175924 | 2.548543 | 1.51E−10 |
| 6 | ENSG00000153283 | CD96 | Tactile | 2.029162 | 0.368458 | 2.460314 | 1.66E−10 |
| 7 | ENSG00000204475 | NCR3 | CD337, NKp30 | 3.472457 | 0.640272 | 2.438804 | 2.31E−10 |
| 8 | ENSG00000085063 | CD59 | MAC-IP, MIRL, | 1.035997 | 0.215186 | 2.265377 | 7.12E−09 |
| | | | protectin | | | | |
| 9 | ENSG00000139626 | ITGB7 | | 2.285445 | 0.50059 | 2.19016 | 8.54E−09 |
| 10 | ENSG00000180644 | PRF1 | | 3.589295 | 0.887169 | 2.016259 | 8.95E−08 |
| 11 | ENSG00000100453 | GZMB | | 11.6194 | 3.515453 | 1.725026 | 4.27E−06 |
| 12 | ENSG00000100385 | IL2RB | | 2.568753 | 0.956632 | 1.424929 | 0.000126 |
| 13 | ENSG00000205809 | KLRC2 | NKG2C, CD159c | 1.419451 | 0.784861 | 0.854636 | 0.026587 |
| 14 | ENSG00000111796 | KLRB1 | CD161 | 18.74844 | 10.45953 | 0.842324 | 0.027995 |
| 15 | ENSG00000150045 | KLRF1 | NKp80 | 0.191285 | 1.177103 | −2.62082 | 4.78E−22 |

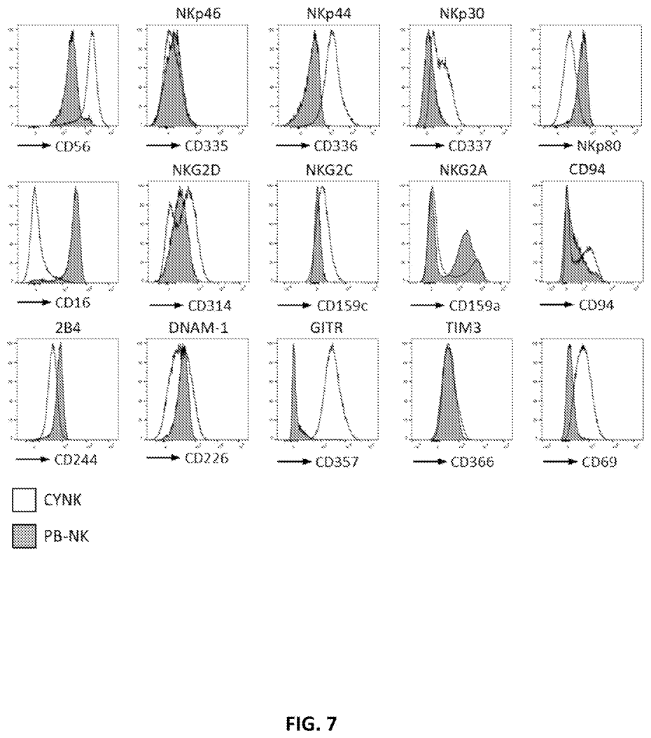

We next analyzed the transcriptional profile of CYNK and PB-NK cells by quantitative real-time PCR (qRT-PCR) focusing on selected NK cell-associated genes that were highly and/or differentially expressed in the scRNAseq dataset (FIG. 7). RNA was extracted from freshly thawed naïve cells post isolation or culture. qRT-PCR demonstrated high expression of CD69, KLRK1 and KLRB1 relative to the housekeeping gene GAPDH in both CYNK and PB-NK cells, whereas, KLRK1 and KLRB1, encoding for NKG2D and CD161/KLRB1, respectively, were significantly higher expressed in PB-NK cells. Significant differential expression of NKp80, encoded by KLRF1 gene, earlier seen by scRNAseq (Table 3), was confirmed by qRT-PCR. Similarly, KLRD1 was higher expressed on PB-NK compared to CYNK cells. Together, the data show higher expression of the inhibitory killer cell lectin-like receptor (KLRB1, KLRD1, KLRF1) expression on PB-NK cells when compared to CYNK cells. The two C-type lectin receptor genes KLRC1 and KLRC2, encoding the inhibitory NKG2A and the activating NKG2C, were higher expressed in CYNK cells. Of the natural cytotoxicity receptors (NCRs), only NCR2 (encoding NKp44) was differentially expressed with high expression in CYNK cells and almost no expression in PB-NK cells. Two co-activating NK cell receptor genes CD244 (2B4) and CD226 (DNAM-1) were slightly higher expressed in PB-NK compared to CYNK cells. Alongside the typical ligand-activated NK cell receptor genes, we also analyzed the expression of FCGR3A encoding an Fc receptor CD16 that is required for antibody-dependent cell-mediated cytotoxicity. Whereas scRNAseq data demonstrated no significant differential expression of FCGR3A, by qRT-PCR it was highly expressed in the PB-NK cells and at a very low level in CYNK cells. The expression of two genes TNFRSF18 and TNFSF10 that were highly differentially expressed by scRNAseq and elevated in the CYNK cluster, were also analyzed by qRT-PCR. The PCR data confirms high expression of these genes encoding for GITR and TRAIL, respectively, on CYNK cells relative to low level expression in PB-NK cells.

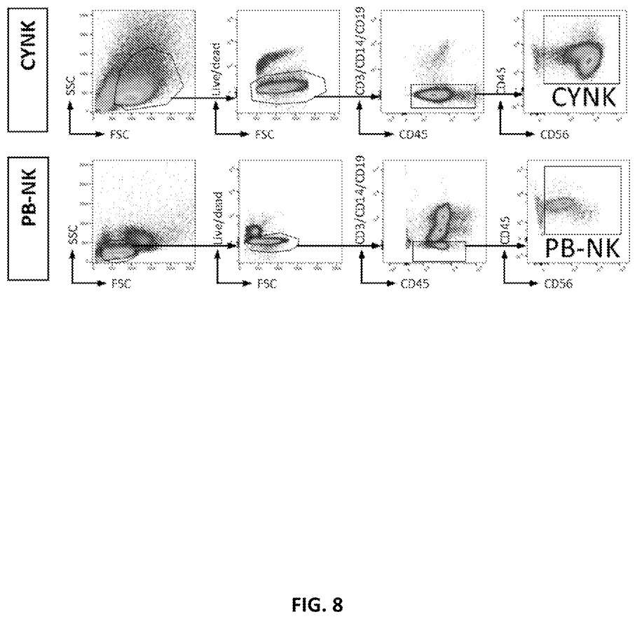

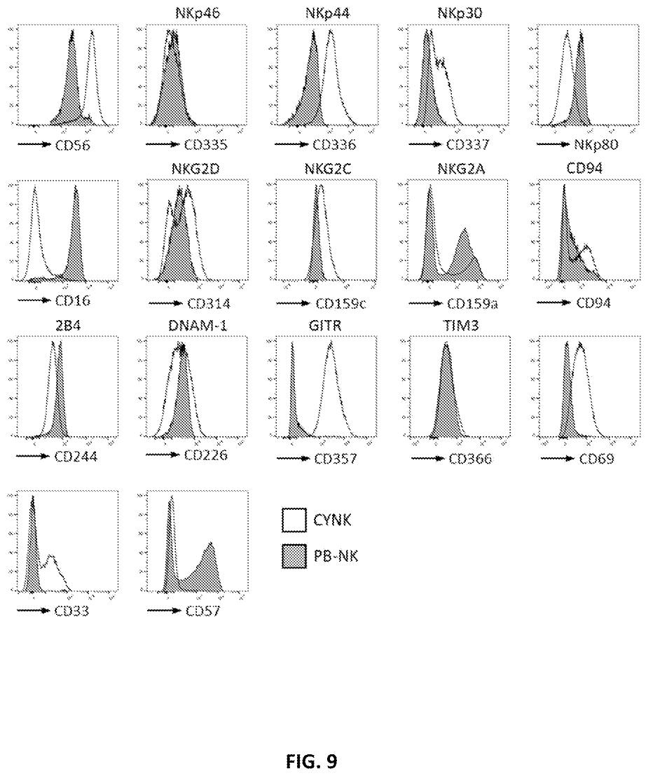

Lastly, we characterized CYNK cells relative to PB-NK by surface protein expression using flow cytometry. Antibodies targeting various NK cell receptors were chosen based on the transcriptional characterization by scRNAseq and qRT-PCR (Tables 1-3, GIG. 6 and FIG. 7). NK cells express high level of the NK cell marker CD56 and lack the expression of T cell, B cell and myeloid cell markers CD3, CD19 and CD14, respectively (FIG. 8). Whereas a majority of PB-NK cells express CD56 at a low level, a small subset of PB-NK cells express CD56 at a level seen in CYNK cells (FIG. 9). NCR analysis demonstrated a high expression of NKp44 in CYNK cells, whereas, NKp44 was expressed at a low level in PB-NK, corresponding well to our transcriptional analysis (FIG. 7). NKp80, on the other hand, was expressed on PB-NK cell and little on CYNK, also confirming the transcriptional data of KLRF1 expression (Table 1 and FIG. 7). CD16 was virtually not expressed on CYNK cells, whereas the majority of PB-NK cells expressed CD16 at a high level. CD16 protein expression, therefore, also corresponds well to transcriptional analysis (Table 1 and FIG. 7). The expression of killer cell lectin-like receptors was comparable between CYNK and PB-NK cells, with CYNK cells demonstrating higher mean fluorescence intensity compared to PB-NK cells for NKG2D, NKG2C, CD94 (NKG2C) and NKG2A. GITR, a checkpoint inhibitor molecule, encoded by TNFRSF18, was not expressed on PB-NK cells but highly on all CYNK cells, correlating well to qRT-PCR data.

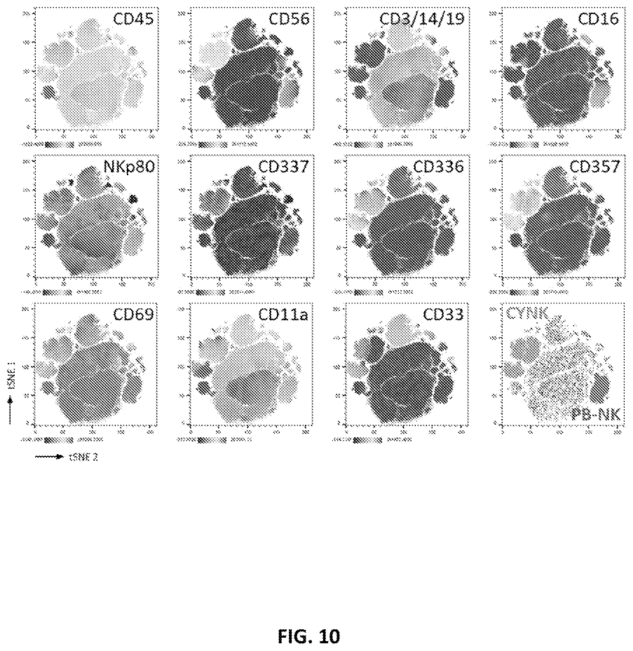

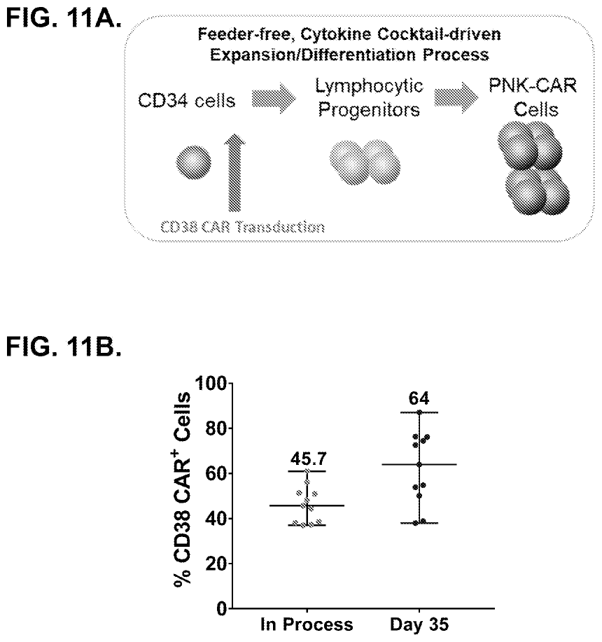

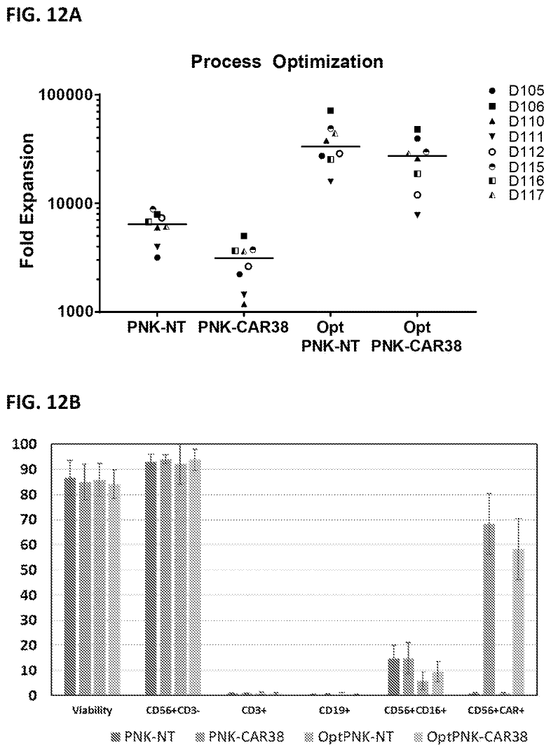

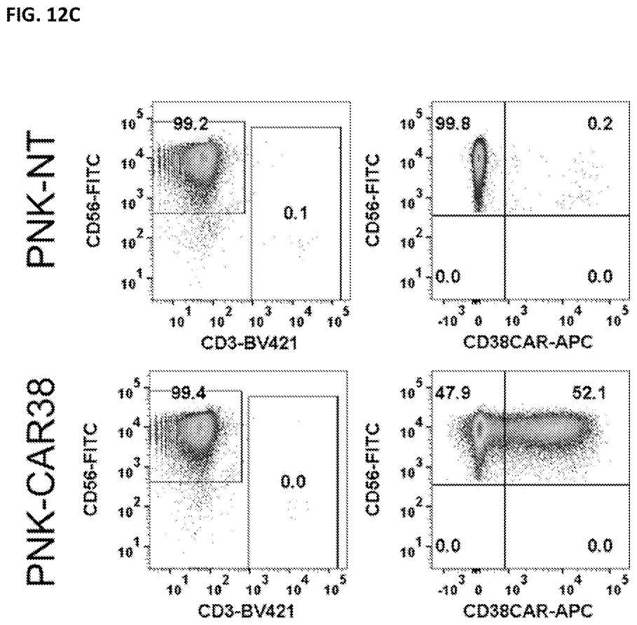

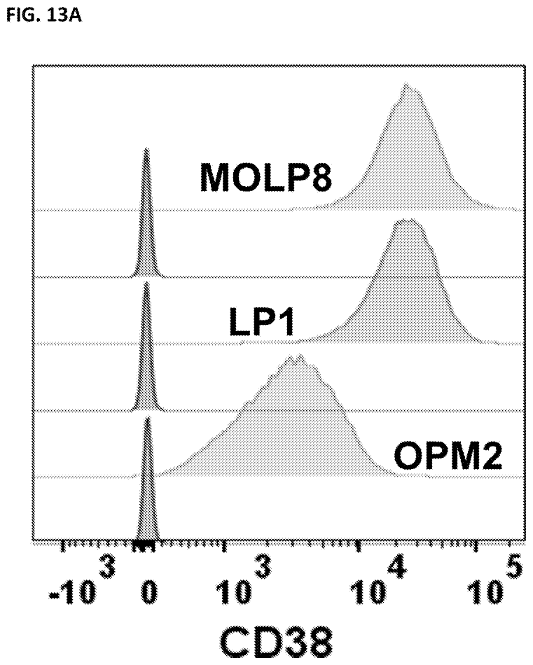

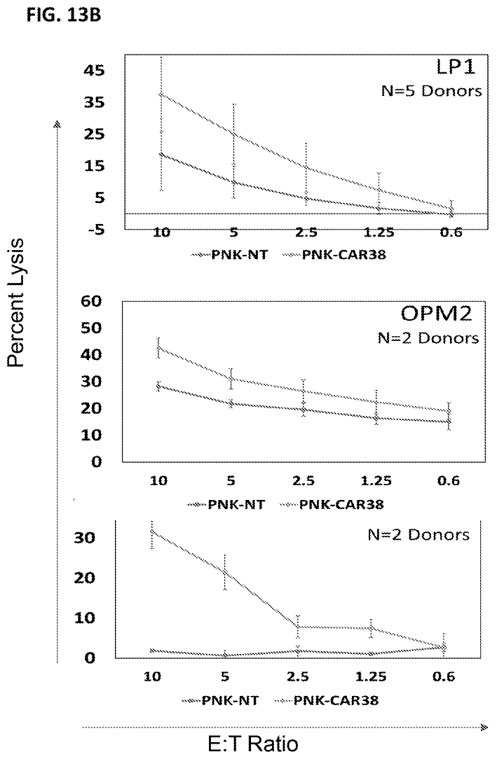

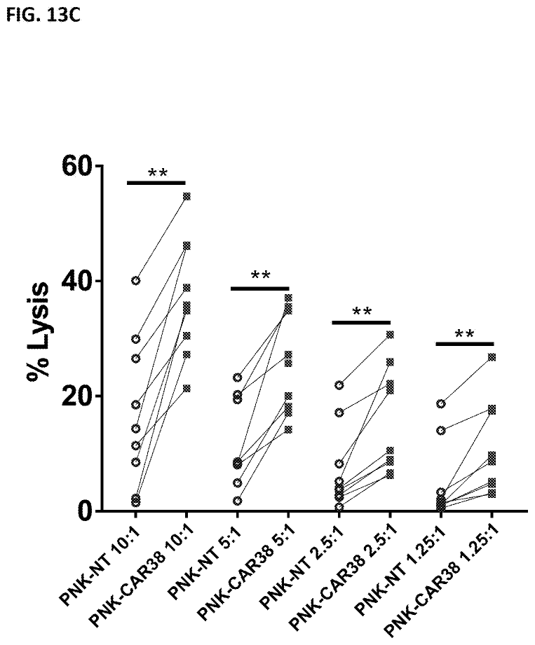

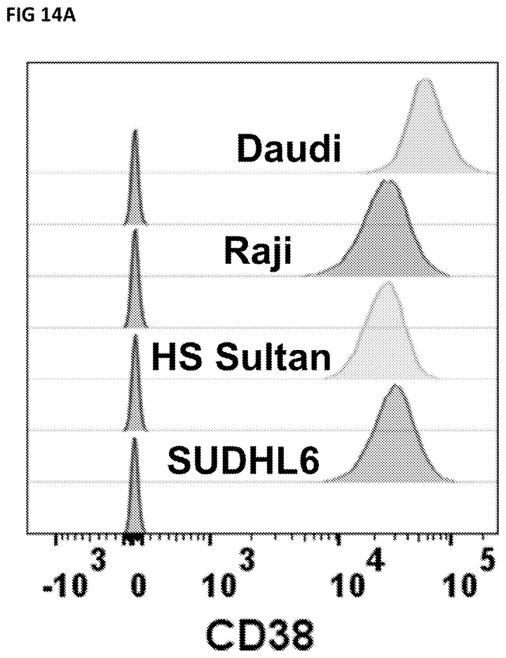

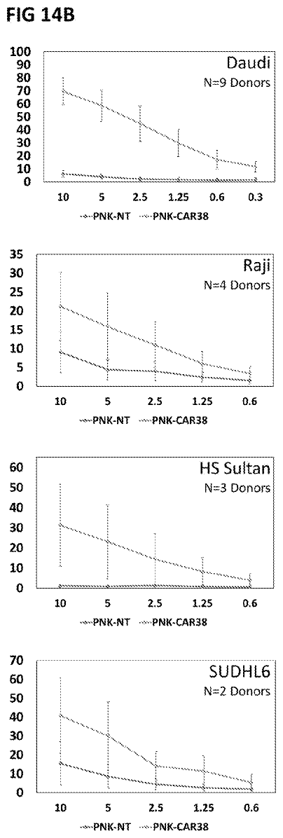

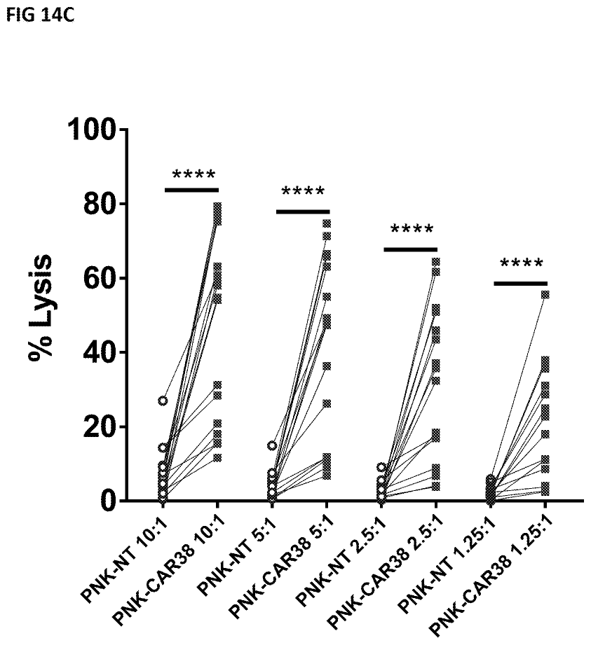

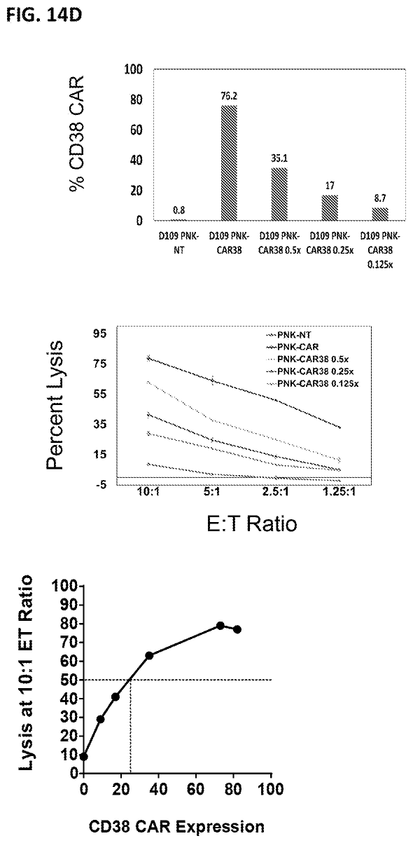

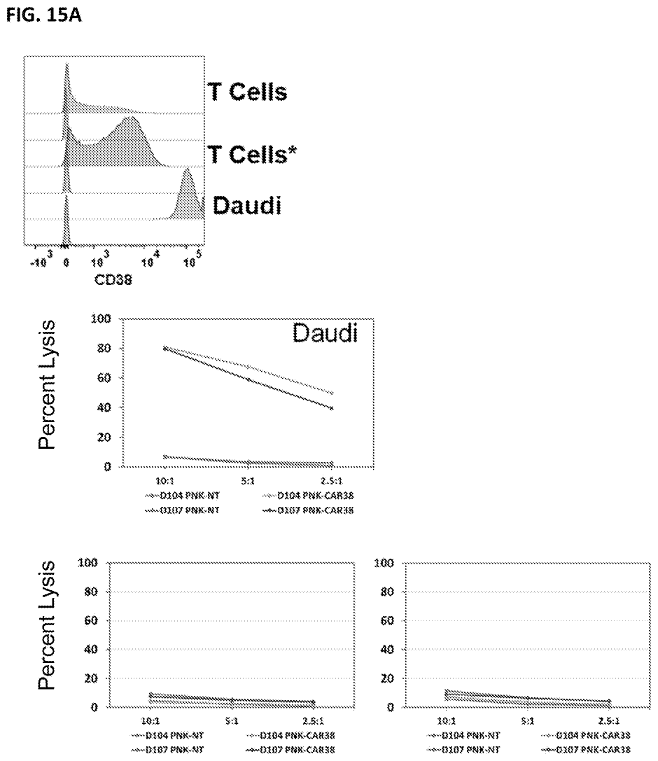

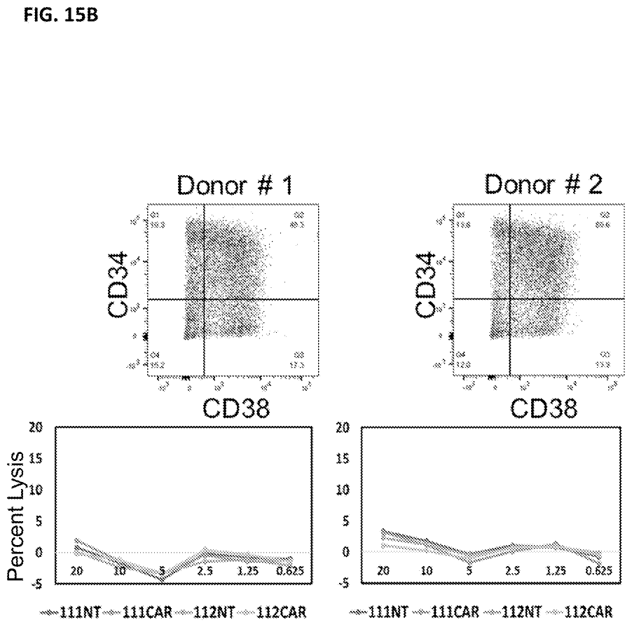

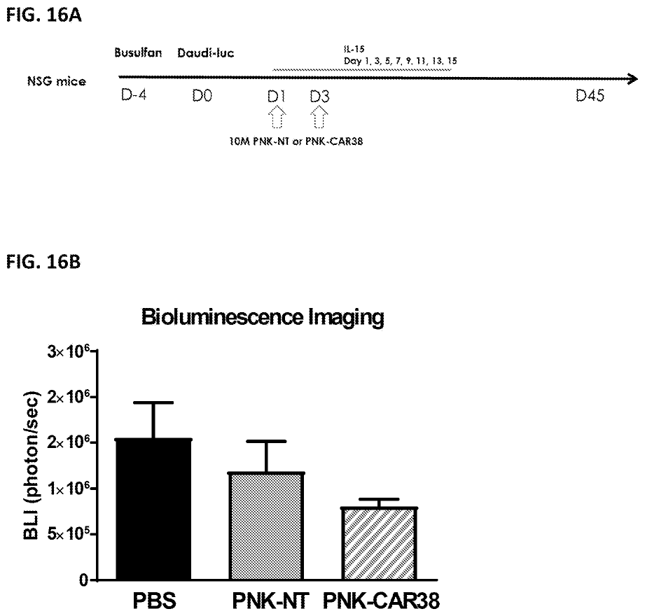

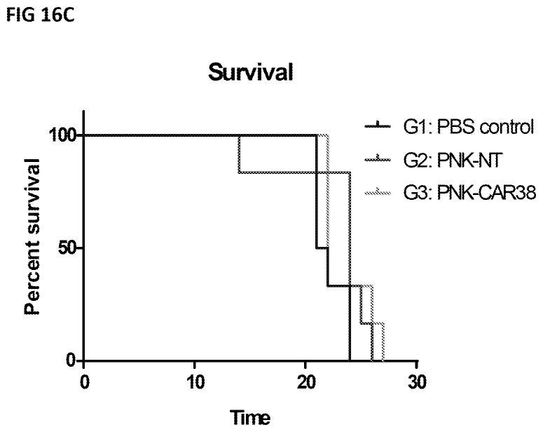

We used the flow cytometry dataset (FIG. 8 and FIG. 9) to perform an unbiased analysis of the surface marker expression on CYNK and PB-NK cell populations (FIG. 10). Antibody-stained CYNK and PBMC cells were mixed for acquisition and analyzed by flow cytometry. It is evident from the tSNE plots that CYNK and PB-NK cells cluster separately from each other and other peripheral blood cells when looking at the localization of CD56- and CD3/CD14/CD19-positive cells on the plot. High expression of NKp44 (CD336) and GITR (CD357) enable the identification of CYNK cells as GITR is virtually not expressed in any cell type in the PBMC subsets. PB-NK cells on the other hand, highly express CD16 and NKp80 that are not expressed on CYNK cells. Altogether, we have identified cell surface markers that allow to distinguish CYNK cells from PB-NK with high confidence.

### 7.5 Example 5: Treatment of Cancers with NK CAR38 Cells

We have demonstrated here that the PNK cells expressing CD38 CAR (PNK-CAR38) have enhanced anti-tumor function against CD38+ lymphoma and multiple myeloma (MM) cell lines in pre-clinical studies. Despite expression of CD38 on healthy lymphocytes and hematopoietic progenitor cells, PNK-CAR38 cells do not exhibit on-target off-tumor cytotoxicity as assessed against healthy CD38+ T cells and CD34⁺CD38⁺ progenitor cells.

Gene Modification and PNK Culture: PNK-CAR38 cells were generated through transduction of human placental CD34+ cells using retroviral vector carrying anti-CD38 CAR (CAR2-anti-CD38 A2; CD38scFv-CD28CD3C) followed by expansion and differentiation to NK cells in presence of cytokines including thrombopoietin, SCF, Flt3 ligand, IL-7, IL-15 and IL-2.

Phenotypic Characterization: The purity of PNK non-transduced cells (PNK-NT) and PNK-CAR38 was determined using flow cytometry. The cells were stained for CD56, CD3, CD19, CD16 and CD38 CAR expression. The viability was assessed using 7AAD staining.

Cytotoxicity Assay: The anti-tumor activity of PNK-NT and PNK-CAR38 cells was assessed against Lymphoma lines—Daudi, Raji, HS Sultan and SUDHL6 and Multiple Myeloma cell lines Molp8, LP1 and OPM2, at various effector to target (E:T) ratios using a 4-hour PKH26 flow cytometry-based method.

In Vivo Anti-Tumor Model: Disseminated Daudi (lymphoma) xenograft model was established in NSG mice. By i.v. inoculation of 3×10⁶ luciferase-expressing Daudi cells on Day 0, followed by IV injection of PBS, PNK-NT or PNK-CAR38 cells (10×10⁶) on Days 1 and 3. Recombinant IL-15 was given to mice every other day for 15 days. Tumor burden was assessed weekly by Bioluminescence Imaging (BLI).

Retroviral transduction of placental CD34+ cells was efficient and generated PNK-CAR38 cells with an average 64% CD38 CAR expression at end of expansion.

A robust expansion was achieved at median of 28,800-fold for PNK-CAR38 cells

PNK-NT and PNK-CAR38 cells showed comparable differentiation and NK cell phenotype.

PNK-CAR38 cells showed significantly higher anti-lymphoma and anti-MM cytolytic function in vitro compared to PNK-NT.

PNK-CAR38 cells with 35% CD38 CAR expression lysed >50% of Daudi cells.

PNK-CAR38 cells did not display on-target off-tumor cytotoxicity against healthy activated T cells and hematopoietic progenitor cells from unrelated donors.

PNK-CAR38 cells showed a 49% reduction in BLI 10 days after PNK-CAR38 cell administration in vivo indicating anti-lymphoma function, but did not demonstrate improvement in survival in the current model.

Further in vivo evaluation of PNK-CAR38 is ongoing.

PNK-CAR38 developed by Celularity is a promising allogeneic approach with low potential for on-target off-tumor toxicity and presents an opportunity for developing CD38 targeted therapy for lymphoma in addition to MM.

### EQUIVALENTS

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes. The citation of any publication is for its disclosure prior to the filing date and should not be construed as an admission that the present invention is not entitled to antedate such publication by virtue of prior invention.

What is claimed is:

1. A method of treating cancer in a human subject comprising administering to the subject an effective amount of placental-derived natural killer cells comprising a CD38 chimeric antigen receptor (CAR) to the subject so as thereby to provide an effective treatment of the cancer in the subject.

2. The method of claim 1, wherein the placental-derived natural killer (NK) cells are CYNK cells.

3. The method of claim 2, wherein the CYNK cells are placental CD34+ cell-derived natural killer (NK) cells.

4. The method of any one of claims 2-3, wherein the CYNK cells are characterized by expression of one or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS which is lower than expression of said markers in peripheral blood natural killer cells and/or expression of one or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 which is higher than expression of said markers in peripheral blood natural killer cells.

5. The method of any one of claims 2-4, wherein the CYNK cells are characterized by expression of one or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS which is lower than expression of said markers in peripheral blood natural killer cells.

6. The method of claim 4 or claim 5, wherein expression of 2, 3, 4, 5, 6, 7, 8, 9, 10, or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS is lower than expression of said markers in peripheral blood natural killer cells.

7. The method of any one of claims 2-6, wherein the CYNK cells are characterized by expression of one or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 which is higher than expression of said markers in peripheral blood natural killer cells.

8. The method of any one of claims 2-7, wherein expression of 2, 3, 4, 5, 6, 7, 8, 9, 10, or more markers selected from the group consisting of NDFIP2, LINC00996, MAL, CCL1, MB, SPINK2, C15orf48, CAMK1, KLRC1, TNF SF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 is higher than expression of said markers in peripheral blood natural killer cells.

9. The natural killer cell of any one of claims 2-8, wherein the CYNK cells are prepared by the methods presented herein.

10. The method of any one of claims 1-9, wherein the cancer is multiple myeloma.

11. The method of any one of claims 1-9, wherein the cancer is a lymphoma.

12. The method of any one of claims 1-11, wherein the CD38 CAR has been introduced into the NK cells by transfection.

13. The method of any one of claims 1-11, wherein the CD38 CAR has been introduced into the NK cells by transduction.

14. The method of claim 13, wherein the CD38 CAR has been introduced into the NK cells by retroviral transduction.

15. The method of claim 13, wherein the CD38 CAR has been introduced into the NK cells by lentiviral transduction.

16. A composition comprising human human placental-derived natural killer cells comprising a CD38 chimeric antigen receptor (CAR) for use in the treatment of a cancer in a subject.

17. Use of a composition comprising human placental-derived natural killer cells comprising a CD38 chimeric antigen receptor (CAR) for use in the manufacture of a medicament for treatment of a cancer in a subject.

18. The composition of claim 16 or use of claim 17, wherein the cancer is multiple myeloma.

19. The composition of claim 16 or use claim 17, wherein the cancer is a lymphoma.

20. The composition or use of any one of claims 16-19, wherein the placental-derived natural killer (NK) cells are CYNK cells.

21. The composition or use of claim 20, wherein the CYNK cells are placental CD34+ cell-derived natural killer (NK) cells.

22. The composition or use of any one of claims 16-21, wherein the CYNK cells are characterized by expression of one or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS which is lower than expression of said markers in peripheral blood natural killer cells and/or expression of one or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 which is higher than expression of said markers in peripheral blood natural killer cells.

23. The composition or use of any one of claims 16-22, wherein the CYNK cells are characterized by expression of one or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS which is lower than expression of said markers in peripheral blood natural killer cells.

24. The composition or use of any one of claims 16-23, wherein expression of 2, 3, 4, 5, 6, 7, 8, 9, 10, or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS is lower than expression of said markers in peripheral blood natural killer cells.

25. The composition or use of any one of claims 16-24, wherein the CYNK cells are characterized by expression of one or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 which is higher than expression of said markers in peripheral blood natural killer cells.

26. The composition or use of any one of claims 16-25, wherein expression of 2, 3, 4, 5, 6, 7, 8, 9, 10, or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 is higher than expression of said markers in peripheral blood natural killer cells.

27. The composition or use of any one of claims 16-26, wherein the CYNK cells are prepared by the methods presented herein.
